(19) United States
(12) Patent Application Publication
(10) Pub. No.: US 2023/0172925 A1
(43) Pub. Date: Jun. 8, 2023

(54) SKIN-CARE COMPOSITIONS AND USES THEREOF

(71) Applicant: AHAVA - DEAD SEA LABORATORIES LTD., Lod (IL)

(72) Inventors: Zeevi MAOR, D.N Arvot Hayarden Dead Sea (IL); Meital PORTUGAL COHEN, Jerusalem (IL); Dror COHEN, D.N the Dead Sea (IL); David BARAK, Modi'in (IL); Yaara Laor COSTA, D.N the Dead Sea (IL); Julia GOROVETZ, Maale Adumim (IL)

(73) Assignee: AHAVA - DEAD SEA LABORATORIES LTD., Lod (IL)

(21) Appl. No.: 17/634,364

(22) PCT Filed: Aug. 13, 2020
(86) PCT No.: PCT/IL2020/050894

§ 371 Date: Feb. 10, 2022

Related U.S. Application Data
(60) Provisional application No. 62/986,698, filed on Mar. 8, 2020.
(60) Provisional application No. 62/886,036, filed on Aug. 13, 2019.

(51) Int. Cl. A61K 31/455; A61K 31/01; A61K 36/05; A61K 36/725; A61K 36/48; A61K 45/06; A61K 8/67; A61K 8/9722; A61K 8/9789; A61Q 19/08

(52) U.S. Cl. CPC A61K 31/455; A61K 31/01; A61K 36/05; A61K 36/725; A61K 36/48; A61K 45/06; A61K 8/675; A61K 8/9722; A61K 8/9789; A61Q 19/08

(57) ABSTRACT

The present disclosure provides compositions including at least one Dead Sea extract, β-carotene and Niacinamide. The present disclosure further provides formulations including the compositions, their uses and methods utilizing same.

### FIELD OF THE INVENTION

This invention relates to compositions comprising Dead Sea extract in combination with β-carotene and Niacinamide and their uses.

### BACKGROUND OF THE INVENTION

Dead Sea water, salts, minerals and mud are well known for their therapeutic efficacy in treating a variety of skin conditions such as psoriasis, atopic dermatitis, acne and other inflammation skin diseases as well as for their cosmetic benefits [1]-[5].

Algae are an especially rich natural source of vitamins and minerals. Algae grown in mineral rich water and in severe environmental conditions are capable of concentrating large amounts of these substances.

Dunaliella alga is a micro alga of saline waters, belonging to Dunaliellaceae family of green alga, named after the Swedish phytoplankton researcher, Dunal [6]-[10].

The species Dunaliella Salina has a length of 8-25 microns and a width of 5-15 microns. Due to these dimensions it is too small to be seen without a microscope. The structure of the Dunaliella Salina is similar to that of other green algae, containing chloroplast, a pyranoid, a nucleus, mitochondria, small vacuoles and golgi bodies. However, differing from other types of algae, Dunaliella Salina cell lacks a cell wall and is encased in a thin elastic membrane. Since it has no cell wall (it behaves like a protoplast), the cell is flexible and can change its volume in response to alteration in osmotic pressure and this phenomenon enables its survival in variety of salinities including extreme levels.

Dunaliella Salina alga has two flagella of equal length and a single cup-shaped chloroplast. Following exposure to sun light this chloroplast accumulates large quantities of β-carotene as droplets at its periphery and consequently the cells color appears orange-red rather than green.

Dunaliella Salina is the richest algal source of β-carotene. The content of β-carotene depends on salinity, temperature and light intensity and varied between 3-10% w/w. The bio-mass of Dunaliella Salina alga is used for cosmetic preparations.

Topical β-carotene is converted into retinyl esters by human epidermis and thus appears as a precursor of epidermal retinol (natural Vitamin A) [11].

Dunaliella Salina extract is known as enhancing the synthesis in fibroblasts of Collagen I, the latter is important for dermal connective tissue [12].

Maor Z. et. al [13]-[14] describe skin care and protection compositions comprising Dead Sea water and Dunaliella Salina extract.

### REFERENCES

[1] Sukenik S., et al., Treatment of psoriatic arthritis at the Dead Sea. J. Rheumatol. 1994, 21, 1305-1309.
[2] S. Halevy., et al. Dead Sea bath salt for the treatment of psoriasis vulgaris: a double-blind controlled study. Journal of the European Academy of Dermatology and Venereology, 1997, 9, 237-242.
[3] Maor Z. and Yehuda S. Skin smoothing effects of Dead Sea minerals: comparative profilometric evaluation of skin surface. International Journal of Cosmetic Science, 1997, 19, 105-110.
[4] Shimon W. Moses, Michael David, Ehud Goldhammer, Asher Tal and Shaul Sukenik. The Dead Sea, A Unique Natural Health Resort. IMAJ, 2006, 8, 483-488.
[5] J. Azmir et al. Techniques for extraction of bioactive compounds from plant materials: A review. Journal of Food Engineering, 2013, 117, 426-436.
[6] M. A. Borowitzka and J. Borowitzka—Micro algal biotechnology, Cambridge University Press, 1988.
[7] C. A. Tukington and J. S. Dover—Skin Deep: an A-Z of skin disorders, treatments and health, Published by Facts On Files Inc. New York, 1996.
[8] Dunaliella—a product manufacturer brochure by: Alban Muller International—Paris, 1999.
[9] A. Ben Amots and M. Avron—In: Algea Biomass, ed. G. Shelef and C. J. Soeder, pp 603-610. Published by Elsevier/North Holland Biochemical Press. Amsterdam, 1980.
[10] Pauline Spolaore et. al., Commercial Applications of Microalgae, Journal of Bioscience and Bioengineering, Vol. 101, No. 2, 87-96, 2006.
[11] Tetsuya Sayo et. al., Lutein, a Nonprovitamin A, Activates the Retinoic Acid Receptor to Induce HAS3-Dependent Hyaluronan Synthesis in Keratinocytes, Biosci. Biotechnol. Biochem., 77 (6), 1282-1286, 2013.
[12] Patrick Stolz and Barbara Obermayer, Manufacturing Microalgae for Skin Care, Cosmetics & Toiletries., volume 120, number 3, March 2005.
[13] U.S. Pat. No. 6,248,340.
[14] Maor Z. et. al., Anti-wrinkle and skin-moisturizing effects of a mineral-algal-botanical complex, J. Cosmet. Sci., 51, 27-36, 2000.
[15] Kong R, Cui Y, Fisher G J, et al., Comparative study of the effects of retinol and retinoic acid on histological, molecular, and clinical properties of human skin J Cosmet Dermatol.; 15(1):49-57, 2016.
[16] Duell E A, Derguini F, Kang S et al., Extraction of human epidermis treated with retinol yields retro-retinoids in addition to free retinol and retinyl esters. J Investig Dermatol; 107: 178-82, 1996.
[17] Subramanian et. al., Gene set enrichment analysis: a knowledge-based approach for interpreting genome-wide expression profiles, PNAS, October 25; 102(43):15545-50, 2005.
[18] Varemo L, Nielsen J, Nookaew I., Enriching the gene set analysis of genome-wide data by incorporating directionality of gene expression and combining statistical hypotheses and methods, Nucleic Acids Research, 41(8), 4378-4391. doi: 10.1093/nar/gkt111., 2013.

Acknowledgement of the above references herein is not to be inferred as meaning that these are in any way relevant to the patentability of the presently disclosed subject matter.

### SUMMARY OF THE INVENTION

The inventors of the present invention have developed an active combination of natural extract originated from one of the most saltiest bodies of water on earth i.e., the Dead Sea, β-carotene and Niacinamide (also known as Niacin and Vitamin B3).

The active combination according to the present invention is a highly mineral rich composition comprising Dead Sea extract.

The active combination according to the present invention comprises an amount of β-carotene and Niacinamide, each of the β-carotene amount and the Niacinamide amount is believe to be sufficient to induce intercellular production of retinol (one of the major forms of Vitamin A) upon topical application of the combination onto the skin, thereby enriching a skin cell with retinol which is an essential nutrient that supports skin function. By enriching a skin cell with retinol, the active combination of the present invention may also enrich the cell with other derivatives of Vitamin A e.g., retinoic acid which is also known of its beneficial contribution to the skin [15]-[16].

The application of the active combination of the present invention onto the skin is advantageously expected to enrich a skin cell with Vitamin A (e.g., retinol and/or retinoic acid and/or retinal) and/or its precursors. Without wishing to be bound by theory, the inventors of the present invention believe that upon topical application of the combination of the present invention, retinol is produced in the cell from its precursor, i.e., β-carotene, with the assistance of the retinol dehydrogenase Co-enzyme Niacinamide. Each of β-carotene and Niacinamide are present in the combinations of the present invention in a sufficient amount to induce the aforementioned production of retinol. The enriched retinol may be further metabolize (e.g., oxidize) to retinoic acid.

The topical use of the active combination of the present invention provides the benefit of avoiding the need to directly apply Vitamin A per-se (e.g., retinol and/or retinal and/or retinoic acid) onto the skin, an action that is known as having severe side effects such as teratogenicity, photo toxicity and skin irritation, depending on the amount of the applied Vitamin A, in particular retinol and retinoic acid.

The active combination of the present invention may further comprise an extract of the Dunaliella alga e.g., Dunaliella Salina micro alga known to be grown in saline waters inter-alia in the almost saturated Dead Sea Water.

In addition to the above noted benefit of the combination according to the present invention, when present, the Dunaliella alga extract e.g., the Dunaliella Salina extract may assist in carrying and delivering the minerals to the skin upon topical application of the combination. Without wishing to be bound by theory, the biomass of the alga may serve as a vehicle for delivering the minerals to the skin, thus, providing the combination with the advantage of enriching a skin cell with both Vitamin A (e.g., retinol) and minerals which are essential ingredients for healthy skin.

As the present application will further disclose, the combinations of the present invention have proven to have skin care and therapeutic attributes, particularly skin related, both protective/preventive and therapeutic.

It is believed that, each of the β-carotene and the Niacinamide are present in the combinations of the present invention in an amount sufficient to provide a therapeutic and/or a cosmetic effect e.g., associated with Vitamin A or derivatives thereof.

For example, the inventors of the present application have surprisingly found that the combination of the present invention comprising Dead Sea water, an extract of Dunaliella Salina, β-carotene and Niacinamide affected the regulation of various genes which are involved in some important biological pathways. The effect of the combination of the present invention was different from the effect observed with the individual ingredients which constitute the combination. Such an effect was observed with the following exemplary non limiting biological pathways: cell cycle (KEGG, Kyoto Encyclopedia of Genes and Genomes), signaling pathways regulating pluripotency of stem cells (KEGG), nucleotide excision repair (KEGG), P53 signaling pathway (KEGG), assembly of collagen fibrils and other multimeric structures (WIKI), Elastic fiber formation (WIKI) and keratinization (WIKI).

Further, the inventors of the present application demonstrated an advantage of the combinations of the present invention, comprising Dead Seawater, an extract of Dunaliella Salina, β-carotene and Niacinamide, over a previously disclosed composition comprising Dead Sea water and Dunaliella Salina extract ([13] and [14]).

The combinations of the present invention comprising Dead Sea water, an extract of Dunaliella Salina, β-carotene and Niacinamide also illustrated a beneficial effect compared to compositions comprising retinol, the latter induced secretion of the cytokine IL-1α upon topical application thereof onto skin explants, both exposed and non-exposed to stress by UVB irradiation. Contrary to the above effect observed with retinol, the combination of the present application attenuated the secretion of the IL-1α cytokine which secretion thereof is related to irritation. Hence, the combination of the present invention provides the cell with a source of Vitamin A such as retinol e.g., via enhancing the production thereof in the cell, without the side effects associate with direct topical application thereof.

Further, the combinations of the present invention comprising Dead Sea water, an extract of Dunaliella Salina, β-carotene and Niacinamide beneficially attenuated the inflammatory cytokine TNFα secretion following exposure of skin explants to stress by UVB irradiation, an effect that was not observed with retinol only.

Furthermore, the combinations of the present invention comprising Dead Sea water, an extract of Dunaliella Salina, β-carotene and Niacinamide beneficially induced the production of hyaluronic acid in human primary fibroblasts cells, an effect which is believe to be without the accompanied side effects associated with retinol.

Thus, the present invention provides in one of its aspects a composition comprising at least one Dead Sea extract, β-carotene and Niacinamide.

In another one of its aspects the present invention provides a composition comprising at least one Dead Sea extract, β-carotene and Niacinamide, wherein the concentration of the β-carotene in the composition is at least about 1 ppm and the concentration of the Niacinamide in the composition is at least about 0.02% w/w.

In another one of its aspects the present invention provides a composition comprising at least one Dead Sea extract, at least one extract of Dunaliella alga, which may be selected from one or both of Dunaliella Salina extract and Dunaliella Bardawill extract, β-carotene and Niacinamide, wherein the concentration of the β-carotene in the composition is at least about 1 ppm and the concentration of the Niacinamide in the composition is at least about 0.02% w/w.

In a particular embodiment according to the present invention the extract of Dunaliella alga is and extract of Dunaliella Salina alga.

In a further one of its aspects the present invention provides a composition comprising about 0.05% w/w to about 5.0% w/w Dead Sea extract, about 1 ppm to about 500 ppm β-carotene (at times about 5 ppm to about 500 ppm) and about 0.02% w/w to about 5.0% w/w Niacinamide.

In another one of its aspects the present invention provides a composition comprising about 0.05% w/w to about 5.0% w/w Dead Sea extract, about 0.1% w/w to about 5.0% w/w Dunaliella extract e.g., Dunaliella Salina extract, about 1 ppm to about 500 ppm 1-carotene (at times about 5 ppm to about 500 ppm) and about 0.02% w/w to about 5.0% w/w Niacinamide.

In another one of its aspects the present invention provides a composition comprising about 0.25% w/w to about 2.5% w/w Dead Sea extract, about 0.5% w/w to about 3.0% w/w Dunaliella extract e.g., Dunaliella Salina extract, about 1 ppm to about 50 ppm 1-carotene (at times about 5 ppm to about 500 ppm) and about 0.05% w/w to about 0.2% w/w Niacinamide.

Yet, in another one of its aspects the present invention provides a composition comprising about 0.5% w/w Dead Sea extract, about 15.78 ppm β-carotene and about 0.10% w/w Niacinamide.

In another one of its aspects the present invention provides a composition comprising about 0.5% w/w Dead Sea extract, about 3.0% w/w Dunaliella extract e.g., Dunaliella Salina extract, about 15.6 ppm β-carotene and about 0.10% w/w Niacinamide.

In a further one of its aspects the present invention provides a composition comprising about 0.5% w/w Dead Sea extract being Dead Sea water (or a concentrate thereof) or an aqueous solution having substantially the same salt and mineral content of the Dead Sea water (or a concentrate thereof), about 3.0% w/w Dunaliella extract being an aqueous extract e.g., Dunaliella Salina aqueous extract, about 15.6 ppm β-carotene and about 0.1% w/w Niacinamide.

In yet another one of its aspects the present invention provides a composition according to the invention for enriching a skin cell with at least one Vitamin A (e.g., one or more of retinol, retinal and retinoic acid), by inducing intercellular production of said at least one Vitamin A upon application of the composition onto at least a region of a skin of a subject.

In another one of its aspects the present invention provides a composition for enriching a skin cell with at least one Vitamin A (e.g., retinol), the composition comprising at least one Dead Sea extract and a combination of β-carotene and Niacinamide, wherein the combination comprises an amount of each of β-carotene and Niacinamide sufficient to induce intercellular production of the at least one Vitamin A (e.g., retinol which may be further metabolize to retinoic acid) upon application of the composition onto at least a region of a skin of a subject, thereby enriching a skin cell with at least one Vitamin A.

In another one of its aspects the present invention provides a composition for enriching a skin cell with at least one Vitamin A (e.g., retinol), the composition comprising at least one Dead Sea extract, at least one Dunaliella extract (which may be selected from one or more of Dunaliella Salina extract and Dunaliella Bardawill extract) and a combination of β-carotene and Niacinamide, wherein the combination comprises an amount of each of β-carotene and Niacinamide sufficient to induce intercellular production of said at least one Vitamin A (e.g., retinol which may be further metabolize to retinoic acid) upon application of said composition onto at least a region of a skin of a subject, thereby enriching a skin cell with at least one Vitamin A.

In another one of its aspects the present invention provides a composition for enriching a skin cell with retinol, the composition comprising at least one Dead Sea extract, at least one Dunaliella Salina extract and a combination of β-carotene and Niacinamide, wherein the combination comprises an amount of each of β-carotene and Niacinamide sufficient to induce intercellular production of retinol upon application of said composition onto at least a region of a skin of a subject, thereby enriching a skin cell with retinol.

In yet a further one of its aspects the present invention provides a composition for enriching a skin cell with retinol, the composition comprising about 0.05% w/w to about 5.0% w/w Dead Sea extract, about 0.10% w/w to about 5.0% w/w Dunaliella Salina extract, about 5 ppm to about 500 ppm β-carotene and about 0.02% w/w to about 5.0% w/w Niacinamide, wherein said composition induces intercellular production of retinol upon application thereof onto at least a region of a skin of a subject, thereby enriching a skin cell with retinol. At times the retinol may be further metabolize to retinoic acid. Thus, the compositions of the invention may further enrich a skin cell with retinoic acid.

Yet, in a further one of its aspects the present invention provides a composition for enriching a skin cell with retinol, the composition comprising about 0.5% Dead Sea extract, about 3.0% w/w Dunaliella Salina extract, about 15.6 ppm R-carotene and about 0.1% w/w Niacinamide, wherein said composition induces intercellular production of retinol upon application thereof onto at least a region of a skin of a subject, thereby enriching a skin cell with retinol.

In another one of its aspects the present invention provides a composition for enriching a skin cell with retinol, the composition comprising about 0.5% Dead Sea extract being Dead Sea water (or a concentrate thereof) or an aqueous solution having substantially the same salt and mineral content of the Dead Sea water (or a concentrate thereof), about 3.0% w/w Dunaliella Salina extract, about 15.6 ppm β-carotene and about 0.1% w/w Niacinamide, wherein said composition induces intercellular production of retinol upon application thereof onto at least a region of a skin of a subject, thereby enriching a skin cell with retinol.

Yet, in a further one of its aspects the present invention provides a method for one or more of protecting and/or improving the state of the skin of a subject and preventing and/or treating imperfections of the skin of a subject in need thereof, wherein the method comprises topically administering (application of) the composition according to the invention onto the skin of the subject.

In a further one of its aspects the present invention provides a method for enriching a skin cell with one or more of Vitamin A (e.g., retinol, retinoic acid and retinal) and/or one or more of Vitamin A precursor, the method comprises topical application of the composition according to the invention onto the skin.

In yet another one of its aspects the present invention provides a method of enriching a skin cell with at least one Vitamin A, the method comprising:

applying onto at least a region of a skin of a subject a composition comprising at least one Dead Sea extract and a combination of β-carotene and Niacinamide, wherein the combination comprises an amount of each of β-carotene and Niacinamide sufficient to induce intercellular production of at least one Vitamin A (e.g., retinol which may be further metabolize to retinoic acid), thereby enriching the skin cell with at least one Vitamin A.

In yet another one of its aspects the present invention provides a method of enriching a skin cell with at least one Vitamin A, the method comprising:

applying onto at least a region of a skin of a subject a composition comprising at least one Dead Sea extract, at least one Dunaliella extract selected from one or more of Dunaliella Salina extract and Dunaliella Bardawill extract and a combination of β-carotene and Niacinamide, wherein the combination comprises an amount of each of β-carotene and Niacinamide sufficient to induce intercellular production of at least one Vitamin A (e.g., retinol which may be further metabolize to retinoic acid), thereby enriching the skin cell with at least one Vitamin A.

In yet another one of its aspects the present invention provides a method of enriching a skin cell with retinol, the method comprising:

applying onto at least a region of a skin of a subject a composition comprising at least one Dead Sea extract, at least one Dunaliella Salina extract and a combination of β-carotene and Niacinamide, wherein the combination comprises an amount of each of β-carotene and Niacinamide sufficient to induce intercellular production of retinol, thereby enriching the skin cell with retinol. At times the retinol may be further metabolize to retinoic acid. Thus, the method of the invention may further enrich a skin cell with retinoic acid.

In a further one of its aspects the present invention provides a method of enriching a skin cell with retinol, the method comprising:

applying onto at least a region of a skin of a subject a composition comprising about 0.1% w/w to about 3.0% w/w Dead Sea extract, about 0.1% w/w to about 5.0% w/w Dunaliella Salina extract, about 5 ppm to about 500 ppm β-carotene and about 0.02% w/w to about 5.0% w/w Niacinamide, wherein said composition induces intercellular production of retinol, thereby enriching the skin cell with retinol.

Yet, in a further one of its aspects the present invention provides a method of enriching a skin cell with retinol, the method comprising:

applying onto at least a region of a skin of a subject a composition comprising about 0.5% w/w Dead Sea extract, about 3.0% w/w Dunaliella Salina extract, about 15.6 ppm β-carotene and about 0.1% Niacinamide, wherein said composition induces intercellular production of retinol, thereby enriching the skin cell with retinol.

In another one of its aspects the present invention provides a method of enriching a skin cell with retinol, the method comprising:

applying onto at least a region of a skin of a subject a composition comprising about 0.5% w/w Dead Sea extract being Dead Sea water (or a concentrate thereof) or an aqueous solution having substantially the same salt and mineral content of the Dead Sea water (or a concentrate thereof), about 3.0% w/w Dunaliella Salina extract, about 15.6 ppm β-carotene and about 0.1% Niacinamide, wherein said composition induces intercellular production of retinol, thereby enriching the skin cell with retinol.

In a further one of its aspects the present invention provides a method of inducing in-vivo production of one or more of Vitamin A (e.g., retinol, retinoic acid and retinal) the method comprising:

application onto at least a region of a skin of a subject a composition according to the invention, thereby inducing production of said one or more of Vitamin A in at least one skin cell of said subject.

Yet in a further one of its aspects the present invention provides a method of inducing in-vivo production of retinol comprising:

application onto at least a region of a skin of a subject a composition comprising at least one Dead Sea Extract, β-carotene and Niacinamide, wherein the amount of the Niacinamide in said composition is sufficient to assist retinol dehydrogenase enzyme in transforming retinal into retinol, thereby inducing production of retinol in at least one skin cell of said subject. At times the retinol may be further metabolize to retinoic acid. Thus, the method may further enrich a skin cell with retinoic acid.

Yet in a further one of its aspects the present invention provides a method of inducing in-vivo production of retinol comprising:

application onto at least a region of a skin of a subject a composition comprising at least one Dead Sea Extract, at least one Dunaliella extract selected from one or more of Dunaliella Salina extract and Dunaliella Bardawill extract, β-carotene and Niacinamide, wherein the amount of the Niacinamide in said composition is sufficient to assist retinol dehydrogenase enzyme in transforming retinal into retinol, thereby inducing production of retinol in at least one skin cell of said subject. At times the retinol may be further metabolize to retinoic acid. Thus, the method may further enrich a skin cell with retinoic acid.

In another one of its aspects the present invention provides a method of inducing in-vivo production of retinol comprising:

application onto at least a region of a skin of a subject a composition comprising at least one Dead Sea Extract, β-carotene and Niacinamide, wherein the amount of the β-carotene in said composition is sufficient to provide a source for retinol and wherein the amount of the Niacinamide in said composition is sufficient to assist retinol dehydrogenase enzyme in transforming retinal into retinol, thereby inducing production of retinol in at least one skin cell of said subject. At times the retinol may be further metabolize to retinoic acid. Thus, the method may further enrich a skin cell with retinoic acid.

In another one of its aspects the present invention provides a method of inducing in-vivo production of retinol comprising:

application onto at least a region of a skin of a subject a composition comprising at least one Dead Sea Extract, at least one Dunaliella Salina extract selected from one or more of Dunaliella Salina extract and Dunaliella Bardawill extract, β-carotene and Niacinamide, wherein the amount of the β-carotene in said composition is sufficient to provide a source for retinol and wherein the amount of the Niacinamide in said composition is sufficient to assist retinol dehydrogenase enzyme in transforming retinal into retinol, thereby inducing production of retinol in at least one skin cell of said subject. At times the retinol may be further metabolize to retinoic acid. Thus, the method may further enrich a skin cell with retinoic acid.

In another one of its aspect the present invention provides the use of a composition according to the invention for the preparation of a topical formulation (e.g., skin-care and/or pharmaceutical) wherein said topical formulation is for enriching a skin cell with at least one Vitamin A derivative (e.g., retinol, retinoic acid, retinal) and/or at least one precursor of Vitamin A.

Yet, in a further one of its aspects the present invention provides skin-care compositions (formulations) and/or pharmaceutical compositions (formulations).

In a further one of its aspects the present invention provides compositions for one or more of protecting and/or improving the state of the skin, and preventing and/or treating imperfections of the skin of a subject.

In another one of its aspects the present invention provides compositions for use in the preparation of skin-care and/or pharmaceutical formulations.

Yet, in a further one of its aspects the present invention provides compositions for treating or preventing at least one disease or disorder e.g., of the skin.

In a further one of its aspect the present invention provides the use of a composition according to the invention for the preparation of a pharmaceutical composition for treating or preventing a disease or disorder e.g., of the skin.

In another one of its aspects the present invention provides one or more of a lotion, an ointment, a gel, a mask, a toner, an essence, a shampoo, a moisturizer, a sunscreen, a cream, a stick, a spray, an aerosol, foam, a paste, a mousse, a solid, semi-solid, or a liquid make-up, a foundation, and an eye make-up comprising the composition according to the invention.

Yet, in a further one of its aspects the present invention provides a method for one or more of protecting and/or improving the state of the skin of a subject and preventing and/or treating imperfections of the skin of a subject in need thereof, wherein the method comprises topically administering (application of) the composition according to the invention onto the skin of the subject.

In another one of its aspects the present invention provides a method for treating or preventing a disease or disorder of the skin of a subject, the method comprises administering to a subject in need thereof a composition according to the invention.

In a further one of its aspects the present invention provides a method for treating and/or preventing one or more disease or disorder, the method comprises administration (e.g., topical) of the composition according to the invention to a subject in need thereof, wherein the disease or disorder are associated with and/or are mediated by and/or are affected by and/or are related to one or more of biological pathways beings selected from cell cycle (KEGG, Kyoto Encyclopedia of Genes and Genomes); signaling pathways regulating pluripotency of stem cells (KEGG); nucleotide excision repair (KEGG); P53 signaling pathway (KEGG); assembly of collagen fibrils and other multimeric structures (WIKI); Elastic fiber formation (WIKI); keratinization (WIKI); or any combination thereof.

In a further one of its aspects the present invention provides a method for treating and/or preventing one or more disease or disorder, the method comprises administration (e.g., topical) of the composition according to the invention to a subject in need thereof, wherein said disease or disorder is associated with and/or is mediated by and/or is affected by and/or is related to one or more of biological pathways beings selected from sphingolipid metabolism (KEGG); NF KAPP B signaling pathway (KEGG); TNT signaling pathway (KEGG); apoptosis (KEGG); base excision repair (KEGG); nod-like receptor signaling pathway (KEGG); Yersinia infection (KEGG); keratinization (WIKI) or any combination thereof.

In a further one of its aspects the present invention provides a method for treating and/or preventing one or more disease or disorder, the method comprises administration (e.g., topical) of the composition according to the invention to a subject in need thereof, wherein the disease or disorder are associated with and/or are mediated by and/or are affected by and/or are related to one or more of biological pathways beings selected from cell cycle (KEGG, Kyoto Encyclopedia of Genes and Genomes); signaling pathways regulating pluripotency of stem cells (KEGG); nucleotide excision repair (KEGG); P53 signaling pathway (KEGG); assembly of collagen fibrils and other multimeric structures (WIKI); Elastic fiber formation (WIKI); keratinization (WIKI); sphingolipid metabolism (KEGG); NF KAPP B signaling pathway (KEGG); TNT signaling pathway (KEGG); apoptosis (KEGG); base excision repair (KEGG); nod-like receptor signaling pathway (KEGG); Yersinia infection (KEGG); or any combination thereof.

In a further one of its aspects the present invention provides a method for affecting the regulation of one or more genes, the method comprises administration (e.g., topical) of the composition according to the invention to a subject in need thereof, wherein said genes are involved in one or more of biological pathways beings selected from cell cycle (KEGG, Kyoto Encyclopedia of Genes and Genomes); signaling pathways regulating pluripotency of stem cells (KEGG); nucleotide excision repair (KEGG); P53 signaling pathway (KEGG); assembly of collagen fibrils and other multimeric structures (WIKI); Elastic fiber formation (WIKI); keratinization (WIKI); sphingolipid metabolism (KEGG); NF KAPP B signaling pathway (KEGG); TNT signaling pathway (KEGG); apoptosis (KEGG); base excision repair (KEGG); nod-like receptor signaling pathway (KEGG); Yersinia infection (KEGG); or any combination thereof. The method may be accompanied with a therapeutic and/or a cosmetic effect as herein disclosed and/or exemplified. Further, the effect of the combination of the active ingredients of the composition may be different from the effect observed with one or more of the individual active ingredients of the composition.

Yet, in a further one of its aspects the present invention provides the composition according to the invention for use in a method of enriching at least a region of a skin of a subject with hyaluronic acid, the method comprises application of a composition according to the invention onto at least a region of a skin of the subject.

In another one of its aspects the present invention provides a method of enriching a skin of a subject with hyaluronic acid, the method comprising applying onto at least a region of a skin of said subject a composition according to the invention.

In another one of its aspect the present invention provides the use of a composition according to the invention for the preparation of a topical formulation (e.g., skin-care and/or pharmaceutical) wherein said topical formulation is for enriching a skin cell with hyaluronic acid.

Yet, in a further one of its aspects the present invention provides the composition according to the invention for use in a method of enriching at least a region of a skin of a subject with hyaluronic acid and at least one Vitamin A, the method comprises application of a composition according to the invention onto at least a region of a skin of the subject.

In another one of its aspects the present invention provides a method of enriching a skin of a subject with hyaluronic acid and at least one Vitamin A, the method comprising applying onto at least a region of a skin of said subject a composition according to the invention.

In another one of its aspect the present invention provides the use of a composition according to the invention for the preparation of a topical formulation (e.g., skin-care and/or pharmaceutical) wherein said topical formulation is for enriching a skin cell with hyaluronic acid and at least one Vitamin A.

The present invention also provides compositions, combinations, extracts, uses and methods as herein defined and exemplified.

The methods and/or uses disclosed herein may be cosmetic methods or uses.

The methods and/or uses disclosed herein may be any one of therapeutic or non-therapeutic methods or uses.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 illustrates the biosynthesis of retinol (Vitamin A1) in the cell.

FIG. 2 illustrates a flow diagram of human skin model experiment in which skin explants exposed to UVB radiation as stressor were topically treated.

FIG. 3 illustrates the viability results as observed in a human skin model experiment with UVB radiation as stressor.

FIG. 4 illustrates the TNFα secretion results as observed in a human skin model experiment with UVB radiation as stressor.

FIG. 5 illustrates the IL-1α secretion results as observed in a human skin model experiment with UVB radiation as stressor.

FIG. 6 illustrates a flow diagram of human skin model experiment in which skin explants not-exposed to UVB radiation as stressor were topically treated.

FIG. 7 illustrates the viability results as observed in a human skin model experiment without UVB radiation as stressor.

FIG. 8 illustrates the TNFα secretion results as observed in a human skin model experiment without UVB radiation as stressor.

FIGS. 9A-9B illustrate the IL-1α secretion results as observed in a human skin model experiment without UVB radiation as stressor.

FIGS. 10A-10B illustrate the averaged expression and the Heatmap, Arrays Clustered by log FC, respectively, observed in a DNA microarray assay.

FIGS. 11A-11B illustrate an MA plot and Box plot, respectively, observed for a β-carotene tested sample in a DNA microarray assay.

FIG. 12 illustrates a Heatmap showing all z-scores for 32 pathways selected from KEGG and wiki-pathways, observed in a DNA microarray assay.

FIG. 13 illustrates the log FC Heatmap for wiki collagen biosynthesis and modified enzymes pathway, observed in a DNA microarray assay.

FIG. 14 illustrates the log FC Heatmap for wiki keratinization pathway, observed in a DNA microarray assay.

FIG. 15 illustrates the Heatmap, Arrays Clustered by log FC observed in a DNA microarray assay comparing the composition according to one embodiment of the invention with prior composition and retinol.

FIG. 16 illustrates a flow diagram of human primary fibroblast cells assay in which the cells were exposed to a complex according to one embodiment of the invention and to the retinol followed by determining the hyaluronic Acid content after the exposure.

FIG. 17 illustrates the hyaluronic Acid observed levels in human primary fibroblast cells assay.

It is noted that in the various figures, when used, * represents p-value <0.05, ** represents p-value <0.01 and *** represents p-value <0.001.

### DETAILED DESCRIPTION OF THE INVENTION

The present invention provides in one of its aspects a composition comprising (as an active combination) at least one Dead Sea extract, β-carotene and Niacinamide.

In some embodiments the concentration of the β-carotene in the composition is at least about 1 ppm and the concentration of the Niacinamide in the composition is at least about 0.02% w/w.

In some embodiments the composition further comprises at least one extract of Dunaliella alga.

In some embodiments the at least one extract of Dunaliella alga is selected from Dunaliella Salina extract, Dunaliella Bardawill extract or a combination thereof.

In some embodiments the composition comprises at least one Dead Sea extract, at least one extract of Dunaliella alga, β-carotene and Niacinamide, wherein the concentration of the β-carotene in the composition is at least about 1 ppm and the concentration of the Niacinamide in the composition is at least about 0.02% w/w.

In some embodiments the composition comprises about 0.05% w/w to about 5.0% w/w Dead Sea extract (e.g., between about 0.20% w/w to about 2.5% w/w Dead Sea extract), about 1 ppm to about 500 ppm β-carotene (at times 1 ppm to 50 ppm, or at times about 5 ppm to about 50 ppm) and about 0.02% w/w to about 5.0% w/w Niacinamide.

In some embodiments the composition may further comprise about 0.1% w/w to about 5.0% w/w Dunaliella extract e.g., Dunaliella Salina extract.

In some embodiments the composition comprises about 0.05% w/w to about 5.0% w/w Dead Sea extract, about 0.1% w/w to about 5.0% w/w Dunaliella extract e.g., Dunaliella Salina extract, about 1 ppm to about 500 ppm β-carotene (at times 1 ppm to 50 ppm, or at times 5 ppm to 50 ppm) and about 0.02% w/w to about 5.0% w/w Niacinamide.

In some embodiments the composition comprises about 0.25% w/w to about 2.5% w/w Dead Sea extract, about 0.5% w/w to about 3.0% w/w Dunaliella extract e.g., Dunaliella Salina extract, about 5 ppm to about 50 ppm β-carotene and about 0.02% w/w to about 5.0% w/w Niacinamide.

In some embodiments the composition comprises about 0.20% w/w to about 2.5% w/w Dead Sea extract, about 0.5% w/w to about 3.0% w/w Dunaliella extract e.g., Dunaliella Salina extract, about 5 ppm to about 50 ppm β-carotene and about 0.04% w/w to about 5.0% w/w Niacinamide.

In some embodiments the composition comprises about 0.5% w/w Dead Sea extract (e.g., DSW), about 3.0% w/w Dunaliella Salina extract, about 15.6 ppm β-carotene and about 0.1% w/w Niacinamide.

In some embodiments the composition comprises about 0.5% w/w Dead Sea extract (e.g., DSW), about 3.0% w/w Dunaliella Salina extract being an aqueous extract, about 15.6 ppm β-carotene and about 0.1% w/w Niacinamide.

In some embodiments the composition comprises about 0.2% w/w Dead Sea extract (e.g., DSW), about 1.2% w/w Dunaliella Salina extract, about 6.24 ppm β-carotene and about 0.04% w/w Niacinamide.

In some embodiments the composition of the present invention is devoid of a plant extract, in particular hydro-soluble plant extract.

In some embodiments the composition of the present invention is devoid of one or both Zizyphus and Trigonella foenum plant extract.

In some embodiments the composition of the present invention is devoid of Zizyphus plant extract.

In some embodiments the composition of the present invention is devoid of Trigonella foenum plant extract.

In some embodiments the composition of the present invention further comprises at least one extract of Dunaliella alga and is devoid of a plant extract, in particular hydro-soluble plant extract.

In some embodiments the composition of the present invention further comprises at least one extract of Dunaliella alga and is devoid of one or both Zizyphus and Trigonella foenum plant extract.

In some embodiments the composition of the present invention further comprises at least one extract of Dunaliella alga and is devoid of Zizyphus plant extract.

In some embodiments the composition of the present invention further comprises at least one extract of Dunaliella alga and is devoid of Trigonella foenum plant extract.

In some embodiments the composition of the present invention is substantially free of any one of retinol, retinoic acid and retinal.

In some embodiments the composition of the present invention is substantially free of one or more of retinol, retinoic acid and retinal.

In some embodiments the composition of the present invention is substantially free of retinol.

In some embodiments the composition of the present invention is substantially free of retinoic acid.

In some embodiments the composition of the present invention is substantially free of retinal.

As used herein, the expression “active combination” refers to the ability of the combination to exert an effect (e.g., one or more of protective/preventive skin-care/therapeutic effect, retinol enrichment effect, hyaluronic acid enrichment effect etc.) as disclosed herein. Neither of the components is regarded as a diluent or excipient.

As used herein the term “Dead Sea extract” refers to one or more natural material, in the form of a single material (e.g., inorganic, organic, salt, etc.) or a mixture of natural materials obtained from the waters of the Dead Sea and/or the mud surrounding the Dead Sea and/or the soil bed of the Dead Sea.

In some embodiments the Dead Sea extract is a mixture of natural materials (e.g., salts, minerals) obtained from the waters of the Dead Sea and/or the mud surrounding the Dead Sea and/or the soil bed of the Dead Sea.

In some embodiments the Dead Sea extract is a mixture of natural materials (e.g., salts, minerals) obtained from the waters of the Dead Sea.

In some embodiments the Dead Sea extract is Dead Sea water (or a concentrate thereof) or an aqueous solution having substantially the same salt and mineral content of the Dead Sea water (or a concentrate thereof).

In some embodiments, the Dead Sea extract is the Dead Sea water.

In some embodiments the composition comprises about 0.05% w/w to about 3.0% w/w Dead Sea water, about 0.10% w/w to about 5.0% w/w Dunaliella Salina extract, about 5 ppm to about 500 ppm β-carotene and about 0.02% w/w to about 5.0% w/w Niacinamide.

In some embodiments the composition comprises about 0.25% w/w to about 3.0% w/w Dead Sea water, about 0.5% w/w to about 3.0% w/w Dunaliella Salina extract, about 5 ppm to about 50 ppm β-carotene and about 0.02% w/w to about 5.0% w/w Niacinamide.

In some embodiments the composition comprises about 0.5% w/w Dead Sea water, about 3.0% w/w Dunaliella Salina extract, about 15.6 ppm β-carotene and about 0.1% w/w Niacinamide.

In some embodiments the composition comprises about 0.5% w/w Dead Sea water, about 3.0% w/w Dunaliella Salina extract being an aqueous extract, about 15.6 ppm β-carotene and about 0.10% w/w Niacinamide.

As used herein the term “Dead Sea water” (herein abbreviated DSW) refers to the saline waters obtained from the Dead Sea (Israel or Jordan) region or an aqueous solution prepared by dissolving Dead Sea minerals in an aqueous medium. The term also encompasses aqueous solutions which simulate such natural solution, namely having at least one parameter substantially identical to that measured for the natural DSW, said parameter being at least one of salt content, at least one of mineral content, salt concentration, concentration of a particular cation or anion, ratio of divalent cations to monovalent cations, TDS (Total Dissolved Salt, w/v), soluble natural substances, and other parameters known to define or characterize natural DSW.

In some embodiments the Dead Sea extract is an aqueous solution having salt and mineral content substantially identical to that measured for the natural DSW.

In some embodiments the Dead Sea extract is an aqueous solution having substantially the same salt (a hypersaline concentration) and mineral content as that of the Dead Sea water.

In some embodiments, the Dead Sea extract is the Dead Sea water which may be obtained directly from the Dead Sea filtered water substantially having the same salt content (a hypersaline concentration) as that of the unfiltered Dead Sea water, or Dead Sea water treated by any one or more of various other methods employed to e.g., remove organic matter and residual contaminants therefrom.

In some embodiments, the Dead Sea extract is an aqueous solution simulating the content of DSW i.e., having substantially identical content as that of DSW.

In some embodiments, the Dead Sea extract is an aqueous solution having substantially identical salts content, minerals content, salts concentration and mineral concentrations as that of DSW.

In some embodiments, the Dead Sea extract is an aqueous solution having substantially identical salts content, minerals content, salts concentration, minerals concentrations, concentration of a particular cation or anion, ratio of divalent cations to monovalent cations, TDS, soluble natural substances and other parameters known to define or characterize natural DSW.

In some embodiments, the Dead Sea extract is an aqueous solution simulating the salt content (a hypersaline concentration) of DSW i.e., having salt content substantially identical to that of DSW.

In some embodiments, the Dead Sea extract is an aqueous solution simulating the mineral content of DSW i.e., having mineral content substantially identical to that of DSW.

In some embodiments, the Dead Sea extract is an aqueous solution simulating the salt content (a hypersaline concentration) and the mineral content of DSW i.e., having salt content substantially identical to that of DSW and mineral content substantially identical to that of DSW.

In some embodiments, the Dead Sea water having:

1. a specific density of 1.25-1.35 g/ml,

2. pH=4.6-5.6 (at 25° C.), and/or

3. less than 100 cfu/g of non-pathogenic microbes.

The Dead Sea water having the above physical characteristics is a concentrated extract of Dead Sea water comprising (among other metal salt ions) Ca⁺², Mg⁺², Na⁺ and K⁺ and high concentrations of anions such as Cl⁻ and Br⁻.

In some embodiments, the DSW is a clear colorless viscous liquid (at 25° C.).

In some embodiments, the concentrations of these ions are, as assessed by a water analysis carried out by the Geological Survey of Israel:
Calcium (Ca⁺²): 35,000-40,000 mg/L
Chloride (Cl⁻): 320,000-370,000 mg/L
Magnesium (Mg⁺²): 92,000-95,000 mg/L
Sodium (Na⁺): 1800-3200 mg/L
Potassium (K⁺): 2,500 mg/L, and
Bromide (Br⁻): 10,000-12,000 mg/L.
   Other minerals may also exist in the waters.

In some embodiments, the Dead Sea Water comprises:
Calcium (Ca⁺²): 35,000-40,000 mg/L
Chloride (Cl⁻): 320,000-370,000 mg/L
Magnesium (Mg⁺²): 92,000-95,000 mg/L
Sodium (Na⁺): 2400-3200 mg/L
Potassium (K⁺): 2,500 mg/L, and
Bromide (Br⁻): 10,000-12,000 mg/L.
   Other minerals may also exist in the waters.

In some embodiments, the Dead Sea Water comprises:
Calcium (Ca⁺²): 5,000-10,000 mg/L
Chloride (Cl⁻): 315,000-360,000 mg/L
Magnesium (Mg⁺²): 100,000-150,000 mg/L
Sodium (Na⁺): 1800-2200 mg/L
Potassium (K⁺): 1,000-2,000 mg/L, and
Bromide (Br⁻): 5,000-10,000 mg/L.
   Other minerals may also exist in the waters.

In some further embodiments, the Dead Sea Water comprises:
Calcium (Ca⁺²) 34,000-40,000 mg/L
Chloride (Cl⁻) 320,000-370,000 mg/L
Magnesium (Mg⁺²) 90,000-95,000 mg/L
Potassium (K⁺) 1,300-2,200 mg/L
Sodium (Na⁺) 1,500-2,800 mg/L
Bromide (Br⁻) 11,000-15,000 mg/L.
   Other minerals may also exist in the waters.

In some embodiments, the Dead Sea Water comprises:
Calcium (Ca⁺²): 38,000 mg/L
Chloride (Cl⁻): 345,000 mg/L
Magnesium (Mg⁺²): 92,500 mg/L
Sodium (Na⁺): 2000 mg/L
Strontium (Sr⁺²): 800 mg/L
Potassium (K⁺): 1,400 mg/L, and
Bromide (Br⁻): 11,500 mg/L.
   Other minerals may also exist in the waters.

In some embodiments, the Dead Sea Water comprises:
Calcium (Ca⁺²): 38,000 mg/L
Chloride (Cl⁻): 345,000 mg/L
Magnesium (Mg⁺²): 92,500 mg/L
Sodium (Na⁺): 2000 mg/L
Strontium (Sr⁺²): 800 mg/L
Potassium (K⁺): 1,400 mg/L, and
Bromide (Br⁻): 11,500 mg/L.
   Other minerals may also exist in the waters.

In some embodiments, the DSW is natural DSW which has undergone pre-treatment, e.g., having been concentrated by allowing water to evaporate, for example through solar evaporation, thereafter reconstituted to afford a solution.

In some embodiments the Dead Sea extract is Dead Sea Water preparation commercially available as “Maris Sal” or “Maris Aqua” or “Aqua, Maris Sal” (AHAVA, Israel) referred to herein below also as “Osmoter”.

In some embodiments, the Dead Sea extract is the Dead Sea water exemplified in Example 1 below.

In some embodiments the Dead Sea extract is Dead Sea mud.

The Dead Sea extract is typically an active fraction having by itself at least one attribute which may be enhanced in a combination with one or more of the Dunaliella alga extract e.g., Dunaliella Salina extract, β-carotene and Niacinamide.

At times, the Dead Sea extract is an active fraction having by itself at least one attribute which may be enhanced in a combination with the β-carotene and the Niacinamide.

At times, the Dead Sea extract is an active fraction having by itself at least one attribute which may be enhanced in a combination with the Dunaliella alga extract e.g., Dunaliella Salina extract and the β-carotene.

At times, the Dead Sea extract is an active fraction having by itself at least one attribute which may be enhanced in a combination with the Dunaliella alga extract e.g., Dunaliella Salina extract and the Niacinamide.

At times, the Dead Sea extract is an active fraction having by itself at least one attribute which may be enhanced in a combination with the Dunaliella alga extract e.g., Dunaliella Salina extract, the β-carotene and the Niacinamide.

In some embodiments the concentration of the Dead Sea extract (e.g., Dead Sea water) in the composition (or formulation) of the invention is at least about 0.05% (w/w). At time is it about 0.05%, 0.06%, 0.07%, 0.08%, 0.09%, 0.10%, 0.15%, 0.20%, 0.25%, 0.30%, 0.35%, 0.40%, 0.45%, 0.50%, 0.55%, 0.60%, 0.65%, 0.70%, 0.75%, 0.80%, 0.85%, 0.90%, 0.95%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%. 3.8%, 3.9%, 4.0%, 4.1%, 4.2%, 4.3%, 4.4%, 4.5%, 4.6%, 4.7%, 4.8%, 4.9% and 5.0%. Any value which is between any one of the above values is within the scope of the present disclosure.

In some embodiments the concentration of the Dead Sea extract (e.g., Dear Sea water) in the composition (or formulation) of the invention is between about 0.05% to about 5.0% w/w.

In some embodiments the concentration of the Dead Sea extract (e.g., Dear Sea water) in the composition (or formulation) of the invention is between about 0.20% to about 2.5% w/w, at times between about 0.25% to about 2.5% w/w.

In some embodiments the concentration of the Dead Sea extract (e.g., Dear Sea water) in the composition (or formulation) of the invention is between about 0.05% to about 3.0% w/w.

In some embodiments the concentration of the Dead Sea extract (e.g., Dear Sea water) in the composition (or formulation) of the invention is between about 0.1% to about 3.0% w/w.

In some embodiments the concentration of the Dead Sea extract (e.g., Dear Sea water) in the composition (or formulation) of the invention is between about 0.25% to about 2.5% w/w.

In some embodiments the concentration of the Dead Sea extract (e.g., Dead Sea water) in the composition (or formulation) of the invention is about 0.5% (w/w).

In some embodiments the concentration of the Dead Sea extract (e.g., Dead Sea water) in the composition (or formulation) of the invention is about 0.2% (w/w).

The term “Dunaliella” or any lingual variations thereof relates to the alga Dunaliella (of the order Volvocales), a genus including a variety of unicellular green alga.

In some embodiments the Dunaliella extract may be any one of Dunaliella Salina extract and Dunaliella Bardawill extract. At times the Dunaliella extract is selected from Dunaliella Salina extract, Dunaliella Bardawill extract or a combination thereof.

In some embodiments the Dunaliella extract is an extract of Dunaliella Salina.

In some embodiments the Dunaliella extract is an extract of Dunaliella Bardawill.

At times, the Dunaliella extract may be an active fraction having by itself at least one attribute which may be enhanced in a combination with one or more of the Dead Sea extract, the β-carotene and the Niacinamide.

In some embodiments the Dunaliella extract may be an active fraction having by itself at least one attribute which may be enhanced in a combination with the Dead Sea extract.

At times, the Dunaliella extract may be an active fraction having by itself at least one attribute which may be enhanced in a combination with the Dead Sea extract (e.g., DSW) and the β-carotene.

At times, the Dunaliella extract may be an active fraction having by itself at least one attribute which may be enhanced in a combination with the Dead Sea extract (e.g., DSW) and the Niacinamide.

At times, the Dunaliella extract may be an active fraction having by itself at least one attribute which may be enhanced in a combination with the Dead Sea extract (e.g., DSW), the β-carotene and the Niacinamide present in the composition of the present invention.

In some embodiments the Dunaliella alga e.g., Dunaliella Salina may be originated from the natural habitats.

In some embodiments the Dunaliella alga e.g., Dunaliella Salina may be originated from the habitats of the Dead Sea.

In some embodiments the Dunaliella Salina extract comprises one or more of the following:

Chlorophyll a chlorophyll b

A range of carotenoids and xanthophylls including:

β-carotene β-10%), a carotene, lutein, violaxanthin

Lipids and fatty acids (6% of dry weight), mainly palmitic, stearic, oleic, linoleic and linolenic (omega 3 and omega 6) acids.

Carbohydrates and polyols: mono and di-sacharides of galactose, glucose, manose, xylose, ribose, rhamnose and polysac-harides.

Trace elements: zinc, copper and selenium.

Alpha tocopherol (vitamin E).

In some embodiments the Dunaliella Salina extract comprises the amino acid content detailed in Table 1.

TABLE 1
Amino Acid contents of Dunaliella Salina extract
according to some embodiments of the invention:

| Amino acids | Gram/100 g protein |
|---|---|
| Alanine | 6.3-12.9 |
| Arginine | 0.9-5.2 |
| Aspartate | 2.5-10.7 |
| Cysteine | Traces |
| Glutamate | 7.9-11.9 |
| Glycine | 9.5-13.3 |
| Histidine | 1.2-8.8 |
| Isoleucine | 2.3-4.2 |
| Leucine | 5.0-17.6 |
| Lysine | 4.8-33.9 |
| Methionine | Traces-1.4 |
| Phenylalanine | 1.4-5.4 |
| Proline | 4.2-8.8 |
| Serine | 6.3-8.6 |
| Threonine | 2.5-6.0 |
| Tyrosine | Traces-1.9 |
| Valine | 3.6-5.8 |

The Dunaliella alga extract e.g., Dunaliella Salina extract may be obtained according to known procedures.

In some embodiments the extraction of the Dunaliella Alga may be a maceration of the alga in a selected solvent system.

In some embodiments the Dunaliella alga extract is an hydrophilic extract.

In some embodiments the Dunaliella alga extract is an aqueous extract.

In some embodiments the Dunaliella Salina alga extract is an extract different from an extract in propylene glycol.

In some embodiments the Dunaliella alga extract may be an extract in one or more of glycerin, propanediol, butylene glycol and the like.

In some embodiments the Dunaliella Salina alga extract is an hydrophobic extract.

In some embodiments the Dunaliella alga extract e.g., Dunaliella Salina extract may be liposoluble extract comprising the alga biomass.

In some embodiments the Dunaliella alga extract e.g., Dunaliella Salina extract may be an aqueous soluble extract comprising the alga biomass.

In some embodiments, the Dunaliella Salina Alga extract is the extract exemplified herein in Example 2 below i.e., the commercial Dunaliella Salina Alga extract PEPHA®-CTIVE (DSM Nutritional Products Ltd):

The extract product identification is as follows (Product code: 50 3592 9):

Appearance: clear, yellowish liquid

pH: 4.5-5.5

Relative density d20/20: 1.000-1.050

Residue on drying: >1.5% m/m

Identity by HPTLC: characteristic pattern of ninhydrin positive components; comparable to reference preparation

Total aerobic mesophile plate count: <100 CFU/ml

Specified microorganisms: not detectable in one milliliter

Solubility—PEPHA®-CTIVE is soluble in water.

Composition—as detailed in Table 2 below.

In some embodiments the concentration of the Dunaliella alga extract e.g., Dunaliella Salina extract in the composition (or formulation) of the invention is at least about 0.1% (w/w). At time is it about 0.1%, 0.2%, 0.3%, 0.4%, 0.5%, 0.6%, 0.7%. 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9% and 5.0%. Any value which is between any one of the above values is within the scope of the present disclosure.

In some embodiments the concentration of the Dunaliella Salina extract in the composition (or formulation) of the invention is about 0.1% w/w to about 5.0% w/w.

In some embodiments the concentration of the Dunaliella alga extract e.g., Dunaliella Salina extract in the composition (or formulation) of the invention is about 0.5% w/w to about 3.0% w/w.

In some embodiments the concentration of the Dunaliella alga extract e.g., Dunaliella Salina extract in the composition (or formulation) of the invention is 3.0% (w/w).

In some embodiments the concentration of the Dunaliella alga extract e.g., Dunaliella Salina extract in the composition (or formulation) of the invention is 1.2% (w/w).

As used herein the term “β-carotene” or any lingual variations thereof is interchangeable with any one of the terms “β-carotene”, “Beta-carotene”, “Beta carotene” or any lingual variations thereof.

At times, the β-carotene may be an active ingredient having by itself at least one attribute which may be enhanced in a combination with one or more of the Dead Sea extract (e.g., DSW), the Dunaliella alga extract and the Niacinamide.

In some embodiments the β-carotene may be an active ingredient having by itself at least one attribute which may be enhanced in a combination with the Dead Sea extract.

At times, the β-carotene may be an active ingredient having by itself at least one attribute which may be enhanced in a combination with the Dead Sea extract (e.g., DSW) and the Niacinamide.

At times, the β-carotene may be an active ingredient having by itself at least one attribute which may be enhanced in a combination with the Dead Sea extract (e.g., DSW) and the Dunaliella alga extract.

At times, the β-carotene may be an active ingredient having by itself at least one attribute which may be enhanced in a combination with the Dead Sea extract (e.g., DSW), the Dunaliella alga extract and the Niacinamide present in the composition of the present invention.

In some embodiments the β-carotene serves as a precursor of Vitamin A e.g., one or more of retinol, retinoic acid and retinal.

It is noted that the Dunaliella Salina alga is known as being rich with β-carotene.

In some embodiments the concentration of the β-carotene in the compositions of the present invention refers to a concentration of a β-carotene which is not originated from the Dunaliella Salina alga extract itself.

In some embodiments the concentration of the β-carotene in the compositions of the present invention refer to a total concentration of a β-carotene originated from the Dunaliella alga extract e.g., Dunaliella Salina alga extract itself and from an additional amount of added β-carotene, the latter being originated from a source different from the Dunaliella alga extract.

In some embodiments the β-carotene is a synthetic β-carotene.

In some embodiments the β-carotene is a non-synthetic β-carotene.

In some embodiments the β-carotene is from a source different from the Dunaliella alga.

In some embodiments the concentration of the β-carotene in the composition (or formulation) of the invention is at least about 1 ppm.

In some embodiments the concentration of the β-carotene in the composition (or formulation) of the invention is between about 1 ppm to about 500 ppm.

In some embodiments the concentration of the β-carotene in the composition (or formulation) of the invention is between about 5 ppm to about 500 ppm.

In some embodiments the concentration of the β-carotene in the composition (or formulation) of the invention is between about 1 ppm to about 50 ppm.

In some embodiments the concentration of the β-carotene in the composition (or formulation) of the invention is between about 5 ppm to about 50 ppm.

In some embodiments the concentration of the β-carotene in the composition (or formulation) of the invention is at least about 1 ppm. At time is it about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 150, 200, 250, 300, 350, 400, 450 and 500 ppm. Any value which is between any one of the above values is within the scope of the present disclosure.

In some embodiments the concentration of the β-carotene in the composition (or formulation) of the invention is from about 1 ppm to about 20 ppm, at times 5 to 20 ppm.

In some embodiments the concentration of the β-carotene in the composition (or formulation) of the invention is from about 1 ppm to about 50 ppm, at times 5 to 50 ppm.

In some embodiments the concentration of the β-carotene in the composition (or formulation) of the invention is about 15.6 ppm e.g., originated from a source different from the Dunaliella alga.

In some embodiments the concentration of the β-carotene in the composition (or formulation) of the invention is about 15.78 ppm e.g., being the sum of β-carotene originated from the Dunaliella alga and a different source.

In some embodiments the concentration of the β-carotene in the composition (or formulation) of the invention is about 6.24 ppm.

As used herein the term “Niacinamide” or any lingual variation thereof is interchangeable with any one of the terms “Niacin” and “Vitamin B3” or any lingual variations thereof.

At times, the Niacinamide may be an active ingredient having by itself at least one attribute which may be enhanced in a combination with one or more of the Dead Sea extract (e.g., DSW), the Dunaliella alga extract e.g., Dunaliella Salina extract and the β-carotene.

In some embodiments the Niacinamide may be an active ingredient having by itself at least one attribute which may be enhanced in a combination with the Dead Sea extract.

At times, the Niacinamide may be an active ingredient having by itself at least one attribute which may be enhanced in a combination with the Dead Sea extract (e.g., DSW), the and the β-carotene.

At times, the Niacinamide may be an active ingredient having by itself at least one attribute which may be enhanced in a combination with the Dead Sea extract (e.g., DSW) and the Dunaliella alga extract e.g., Dunaliella Salina extract.

At times, the Niacinamide may be an active ingredient having by itself at least one attribute which may be enhanced in a combination with the Dead Sea extract (e.g., DSW), the Dunaliella alga extract e.g., Dunaliella Salina extract and the β-carotene.

In some embodiments the Niacinamide serves as a Co-enzyme of retinol dehydrogenase.

In some embodiments the Niacinamide is synthetic.

In some embodiments the Niacinamide is non-synthetic.

In some embodiments the Niacinamide is from a source different from a plant extract.

In some embodiments the Niacinamide is not originated from one or both of Zizyphus and Trigonella foenum plant extract.

In some embodiments the Niacinamide is not originated from Zizyphus plant extract.

In some embodiments the Niacinamide is not originated from Trigonella foenum plant extract.

In some embodiments the Niacinamide is from a source different from a Dunaliella alga.

In some embodiments the Niacinamide is not originated from a Dunaliella alga.

In some embodiments the concentration of the Niacinamide in the composition (or formulation) of the invention is at least about 0.02% (w/w). At time is it about 0.02%, 0.03%, 0.04%, 0.05%, 0.06%, 0.07%, 0.08%, 0.09%, 0.1%, 0.2%. 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3% 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9% and 5.0%. Any value which is between any one of the above values is within the scope of the present disclosure.

In some embodiments the concentration of the Niacinamide in the composition (or formulation) of the invention is about 0.02% w/w to about 5.0% w/w.

In some embodiments the concentration of the Niacinamide in the composition (or formulation) of the invention is about 0.04% w/w to about 5.0% w/w.

In some embodiments the concentration of the Niacinamide in the composition (or formulation) of the invention is about 0.04% w/w to about 0.2% w/w.

In some embodiments the concentration of the Niacinamide in the composition (or formulation) of the invention is about 0.05% w/w to about 5.0% w/w.

In some embodiments the concentration of the Niacinamide in the composition (or formulation) of the invention is about 0.05% w/w to about 0.2% w/w.

In some embodiments the concentration of the Niacinamide in the composition (or formulation) of the invention is 0.1% (w/w).

In some embodiments the concentration of the Niacinamide in the composition (or formulation) of the invention is 0.04% (w/w).

In another one of its aspects the present invention provides a composition according to the invention for enriching a skin cell with at least one form of Vitamin A (e.g., retinol) by inducing intercellular production of said form upon application of the composition onto at least a region of a skin of a subject.

Vitamin A is an essential nutrient that supports skin, eye, and reproductive health, and immune function [15]-[16]. There are two types of Vitamin A: retinoids (preformed Vitamin A) and carotenoids (Provitamin A). Both types are converted to retinol by the liver. There, it's either stored or transported by the lymphatic system to cells throughout the body.

In some embodiments of the present invention Vitamin A is one or more of the aforementioned two types of Vitamin A.

In some embodiments of the present invention Vitamin A is a retinoid.

In some embodiments of the present invention Vitamin A is retinol also known as Vitamin A1.

In some embodiments of the present invention Vitamin A is retinoic acid.

In some embodiments of the present invention Vitamin A is retinal.

In some embodiments of the present invention Vitamin A is selected from retinol, retinoic acid, retinal or any combination thereof.

In some embodiments of the present invention Vitamin A is selected from retinol, retinoic acid or any combination thereof.

In some embodiments of the present invention Vitamin A is a carotenoid.

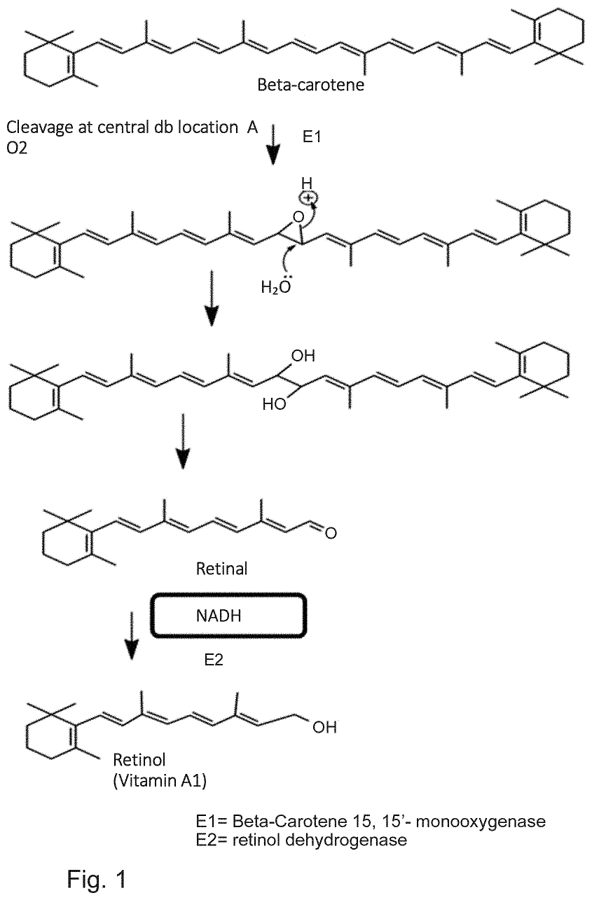

FIG. 1 illustrates the biosynthesis of Vitamin A1 (retinol) in the cell. Beta-carotene undergoes cleavage by Beta-carotene 15, 15′-monooxygenase (referred to as E1 in FIG. 1) to produce Retianal which is then transformed into Vitamin A1 by retinol dehydrogenase enzyme (referred to as E2 in FIG. 1) with NAD Co-A as co-enzyme, also recognized as Niacinamide, Niacin and Vitamin B3.

The compositions of the present invention may assist the skin with providing it with a source of β-carotene, which is a precursor of Vitamin A1, in combination with Niacinamide that assists in transforming the retinal (produced from β-carotene) into retinol. The resulted retinol (or retinoic acid which is an oxidized form of retinol) produced in the skin cell may illustrate safe activity e.g., without the side effects associate with direct application of retinol and/or retinoic acid onto the skin.

The combined amounts of each of β-carotene and Niacinamide in the composition of the present invention provides a combination sufficient to induce intercellular production of one or more of retinol and retinoic acid upon application of the composition onto at least a region of a skin of a subject.

Thus, in another one of its aspects the present invention provides a composition for enriching a skin cell with at least one Vitamin A, the composition comprising at least one Dead Sea extract, and a combination of β-carotene and Niacinamide, wherein the combination comprises an amount of each of β-carotene and Niacinamide sufficient to induce intercellular production of at least one Vitamin A upon application of the composition onto at least a region of a skin of a subject, thereby enriching a skin cell with at least one Vitamin A.

In some embodiments the composition may further comprise at least one Dunaliella alga extract.

In some embodiments the at least one Dunaliella alga extract may be Dunaliella Salina extract, Dunaliella Bardawill extract or a combination thereof.

In some embodiments the composition comprises at least one Dead Sea extract, at least one Dunaliella alga extract (e.g., Dunaliella Salina extract and/or Dunaliella Bardawill extract) and a combination of β-carotene and Niacinamide, wherein the combination comprises an amount of each of β-carotene and Niacinamide sufficient to induce intercellular production of at least one Vitamin A upon application of the composition onto at least a region of a skin of a subject, thereby enriching a skin cell with at least one Vitamin A.

In some embodiments the concentration of the β-carotene in the composition is at least about 1 ppm and the concentration of the Niacinamide in the composition is at least about 0.02% w/w.

In some embodiments the concentration of the β-carotene in the composition and the concentration of the Niacinamide in the composition are as herein disclosed and/or exemplified.

In some embodiments the compositions and the constituents thereof are as herein disclosed and/or exemplified.

In some embodiments the concentration of the β-carotene in the composition and the concentration of the Niacinamide in the composition sufficient to induce intercellular production of at least one Vitamin A (e.g., retinol which may be further metabolize to retinoic acid) are as herein disclosed and/or exemplified.

In some embodiments the composition comprises about 0.25% w/w to about 2.5% w/w Dead Sea extract, about 0.5% w/w to about 3.0% w/w Dunaliella Salina extract, about 1 ppm to about 50 ppm β-carotene and about 0.05% w/w to about 0.2% w/w Niacinamide.

In some embodiments the composition comprises about 0.20% w/w to about 2.5% w/w Dead Sea extract, about 0.5% w/w to about 3.0% w/w Dunaliella Salina extract, about 1 ppm to about 50 ppm β-carotene and about 0.04% w/w to about 0.2% w/w Niacinamide.

In some embodiments the composition comprises about 0.25% w/w to about 2.5% w/w Dead Sea extract, about 0.5% w/w to about 3.0% w/w Dunaliella Salina extract, about 5 ppm to about 50 ppm β-carotene and about 0.05% w/w to about 0.2% w/w Niacinamide.

In some embodiments the composition comprises about 0.20% w/w to about 2.5% w/w Dead Sea extract, about 0.5% w/w to about 3.0% w/w Dunaliella Salina extract, about 5 ppm to about 50 ppm β-carotene and about 0.04% w/w to about 0.2% w/w Niacinamide.

In some embodiments the composition comprises about 0.5% Dead Sea extract, about 3.0% w/w Dunaliella Salina extract, about 15.6 ppm β-carotene and about 0.1% w/w Niacinamide.

In some embodiments the composition comprises about 0.2% w/w Dead Sea extract, about 1.2% w/w Dunaliella Salina extract, about 6.24 ppm β-carotene and about 0.04% w/w Niacinamide.

In some embodiments the composition of the invention is for enriching a skin cell with retinol, the composition comprising at least one Dead Sea extract, and a combination of β-carotene and Niacinamide, wherein the combination comprises an amount of each of β-carotene and Niacinamide sufficient to induce intercellular production of retinol upon application of the composition onto at least a region of a skin of a subject, thereby enriching a skin cell with retinol. At times the composition may further comprise at least one Dunaliella Salina extract.

The term “enriching” or any lingual variation thereof mentioned in connection with Vitamin A is envisaged as enriching a skin cell with an amount of Vitamin A e.g., retinol above an amount naturally produced in the skin cell or above a zero amount e.g., when no production of Vitamin A (e.g., retinol) occurs in the cell.

At times, when no natural production of Vitamin A (e.g., retinol) occurs in the skin cell e.g., due to lack of Vitamin A precursors e.g., β-carotene and/or lack of enzyme/s activity and/or lack of co-enzyme/s activity, topical application of the compositions of the present invention may stimulate the production of Vitamin A (e.g., retinol) in the skin cell. To this end, the term “stimulate” or any lingual variations thereof may be envisaged as initiating the production of Vitamin A e.g., retinol.

In some embodiments the composition of the invention is for enriching a skin cell with retinol, the composition comprising at least one Dead Sea extract, β-carotene and Niacinamide, wherein said composition induces intercellular production of retinol upon application thereof onto at least a region of a skin of a subject, thereby enriching a skin cell with retinol. At times the composition may further comprise at least one Dunaliella Salina extract. In some embodiments the retinol may be further metabolize to retinoic acid. To this end, the composition may further enrich a skin cell with retinoic acid.

In some embodiments the composition of the invention is for enriching a skin cell with retinol, wherein the composition comprises about 0.05% w/w to about 5.0% w/w Dead Sea extract, about 0.1% w/w to about 5.0% w/w Dunaliella Salina extract, about 5 ppm to about 500 ppm β-carotene and about 0.02% w/w to about 5.0% w/w Niacinamide, wherein said composition induces intercellular production of retinol upon application thereof onto at least a region of a skin of a subject, thereby enriching a skin cell with retinol. In some embodiments the retinol may be further metabolize to retinoic acid. To this end, the composition may further enrich a skin cell with retinoic acid.

In some embodiments the composition of the invention is for enriching a skin cell with retinol, wherein the composition comprises about 0.5% Dead Sea extract, about 3.0% w/w Dunaliella Salina extract, about 15.6 ppm β-carotene and about 0.1% w/w Niacinamide, wherein said composition induces intercellular production of retinol upon application thereof onto at least a region of a skin of a subject, thereby enriching a skin cell with retinol.

In some embodiments the composition of the invention is for enriching a skin cell with retinol, wherein the composition comprises about 0.5% Dead Sea extract being Dead Sea water (or a concentrate thereof) or an aqueous solution having substantially the same salt and mineral content of the Dead Sea water (or a concentrate thereof), about 3.0% w/w Dunaliella Salina extract, about 15.6 ppm β-carotene and about 0.1% w/w Niacinamide, wherein said composition induces intercellular production of retinol upon application thereof onto at least a region of a skin of a subject, thereby enriching a skin cell with retinol.

In another one of its aspect the present invention provides the use of a composition according to the invention for the preparation of a topical formulation (e.g., skin-care and/or pharmaceutical) wherein said topical formulation is for enriching a skin cell with at least one Vitamin A e.g., retinol and/or at least one Vitamin A precursor.

In a further one of its aspects the present invention provides a method for enriching a skin cell with at least one Vitamin A e.g., retinol and/or at least one Vitamin A precursor, the method comprises topical application of the composition according to the invention onto the skin.

In yet another one of its aspects the present invention provides a method of enriching a skin cell with at least one Vitamin A, the method comprising:

applying onto at least a region of a skin of a subject a composition comprising at least one Dead Sea extract and a combination of β-carotene and Niacinamide, wherein the combination comprises an amount of each of β-carotene and Niacinamide sufficient to induce intercellular production of at least one Vitamin A (e.g., retinol which may be further metabolize to retinoic acid), thereby enriching the skin cell with at least one Vitamin A.

In some embodiments the method comprises applying onto at least a region of a skin of a subject a composition according to the invention, which at times may further comprise at least one Dunaliella alga extract.

In some embodiments the composition comprises at least one Dead Sea extract, at least one Dunaliella alga extract (e.g., Dunaliella Salina extract and/or Dunaliella Bardawill extract) and a combination of β-carotene and Niacinamide.

In some embodiments the concentration of the β-carotene in the composition is at least about 1 ppm and the concentration of the Niacinamide in the composition is at least about 0.02% w/w.

In some embodiments the concentration of the β-carotene in the composition and the concentration of the Niacinamide in the composition are as herein disclosed and/or exemplified.

In some embodiments the compositions and the constituents thereof are as herein disclosed and/or exemplified.

In some embodiments the composition comprises about 0.25% w/w to about 2.5% w/w Dead Sea extract, about 0.5% w/w to about 3.0% w/w Dunaliella Salina extract, about 1 ppm to about 50 ppm β-carotene and about 0.05% w/w to about 0.2% w/w Niacinamide.

In some embodiments the composition comprises about 0.20% w/w to about 2.5% w/w Dead Sea extract, about 0.5% w/w to about 3.0% w/w Dunaliella Salina extract, about 1 ppm to about 50 ppm β-carotene and about 0.04% w/w to about 0.2% w/w Niacinamide.

In some embodiments the composition comprises about 0.25% w/w to about 2.5% w/w Dead Sea extract, about 0.5% w/w to about 3.0% w/w Dunaliella Salina extract, about 5 ppm to about 50 ppm β-carotene and about 0.05% w/w to about 0.2% w/w Niacinamide.

In some embodiments the composition comprises about 0.20% w/w to about 2.5% w/w Dead Sea extract, about 0.5% w/w to about 3.0% w/w Dunaliella Salina extract, about 5 ppm to about 50 ppm β-carotene and about 0.04% w/w to about 0.2% w/w Niacinamide.

In some embodiments the composition comprises about 0.5% Dead Sea extract, about 3.0% w/w Dunaliella Salina extract, about 15.6 ppm β-carotene and about 0.1% w/w Niacinamide.

In some embodiments the composition comprises about 0.2% w/w Dead Sea extract, about 1.2% w/w Dunaliella Salina extract, about 6.24 ppm β-carotene and about 0.04% w/w Niacinamide.

In some embodiments the method of enriching a skin cell with retinol, comprises:

applying onto at least a region of a skin of a subject a composition comprising at least one Dead Sea extract, at least one Dunaliella Salina extract and a combination of β-carotene and Niacinamide, wherein the combination comprises an amount of each of β-carotene and Niacinamide sufficient to induce intercellular production of retinol, thereby enriching the skin cell with retinol.

In some embodiments the method comprises applying onto at least a region of a skin of a subject a composition comprising about 0.1% w/w to about 3.0% w/w Dead Sea extract, about 0.1% w/w to about 5.0% w/w Dunaliella Salina extract, about 5 ppm to about 500 ppm β-carotene and about 0.05% w/w to about 5.0% w/w Niacinamide, wherein said composition induces intercellular production of retinol, thereby enriching the skin cell with retinol.

In some embodiments the method comprises applying onto at least a region of a skin of a subject a composition comprising about 0.5% w/w Dead Sea extract, about 3.0% w/w Dunaliella Salina extract, about 15.6 ppm β-carotene and about 0.1% Niacinamide, wherein said composition induces intercellular production of retinol, thereby enriching the skin cell with retinol.

In some embodiment the method comprises applying onto at least a region of a skin of a subject a composition comprising about 0.5% w/w Dead Sea extract being Dead Sea water (or a concentrate thereof) or an aqueous solution having substantially the same salt and mineral content of the Dead Sea water (or a concentrate thereof), about 3.0% w/w Dunaliella Salina extract, about 15.6 ppm β-carotene and about 0.1% Niacinamide, wherein said composition induces intercellular production of retinol, thereby enriching the skin cell with retinol.

In a further one of its aspects the present invention provides a method of inducing in-vivo production of one or more of Vitamin A (e.g., retinol, retinoic acid and retinal) the method comprising:

application onto at least a region of a skin of a subject a composition according to the invention, thereby inducing production of said one or more of Vitamin A in at least one skin cell of said subject.

In a further one of its aspects the present invention provides a method of inducing in-vivo production of retinol the method comprising:

application onto at least a region of a skin of a subject a composition according to the invention, thereby inducing production of retinol in at least one skin cell of said subject.

Yet in a further one of its aspects the present invention provides a method of inducing in-vivo production of retinol comprising:

application onto at least a region of a skin of a subject a composition comprising at least one Dead Sea extract, at least one Dunaliella Salina extract, β-carotene and Niacinamide, wherein the amount of the Niacinamide in said composition is sufficient to assist retinol dehydrogenase enzyme in transforming retinal into retinol, thereby inducing production of retinol in at least one skin cell of said subject.

In some embodiments the amount of the Niacinamide in the composition sufficient to assist retinol dehydrogenase enzyme in transforming retinal into retinol is as herein disclosed and/or exemplified.

In another one of its aspects the present invention provides a method of inducing in-vivo production of retinol comprising:

application onto at least a region of a skin of a subject a composition comprising at least one Dead Sea Extract, at least one Dunaliella Salina extract, β-carotene and Niacinamide, wherein the amount of the β-carotene in said composition is sufficient to provide a source for retinol and wherein the amount of the Niacinamide in said composition is sufficient to assist retinol dehydrogenase enzyme in transforming retinal into retinol, thereby inducing production of retinol in at least one skin cell of said subject.

In some embodiments the amount of the β-carotene in the composition sufficient to provide a source for retinol and the amount of the Niacinamide in the composition sufficient to assist retinol dehydrogenase enzyme in transforming retinal into retinol are as herein disclosed and/or exemplified.

In another one of its aspect the present invention provides the use of a composition according to the invention for the preparation of a topical formulation (e.g., skin-care and/or pharmaceutical) wherein said topical formulation is for enriching a skin cell with hyaluronic acid.

Yet, in a further one of its aspects the present invention provides the composition according to the invention for use in a method of enriching at least a region of a skin of a subject with hyaluronic acid, the method comprises application of a composition according to the invention onto at least a region of a skin of the subject.

In another one of its aspects the present invention provides a method of enriching a skin of a subject with hyaluronic acid, the method comprising applying onto at least a region of a skin of said subject a composition according to the invention.

Yet, in a further one of its aspects the present invention provides the composition according to the invention for use in a method of enriching at least a region of a skin of a subject with hyaluronic acid and at least one Vitamin A, the method comprises application of a composition according to the invention onto at least a region of a skin of the subject.

In another one of its aspects the present invention provides a method of enriching a skin of a subject with hyaluronic acid and at least one Vitamin A, the method comprising applying onto at least a region of a skin of said subject a composition according to the invention.

In another one of its aspect the present invention provides the use of a composition according to the invention for the preparation of a topical formulation (e.g., skin-care and/or pharmaceutical) wherein said topical formulation is for enriching a skin cell with hyaluronic acid and at least one Vitamin A.

Yet, in a further one of its aspects the present invention provides skin-care compositions (formulations) and/or pharmaceutical compositions (formulations).

In some embodiments, the composition of the present invention is a cosmetic composition. In other embodiments, the composition of the present invention is a pharmaceutical composition. In further embodiments, the pharmaceutical composition is for topical application.

In some embodiments, the composition is a synergistic composition.

The compositions of the present invention may be made into a wide variety of product forms suitable for, e.g., topical administration onto the skin of a subject. Non-limiting examples are a lotion, an ointment, a gel, a mask, a toner, an essence, a shampoo, a moisturizer, a sunscreen, a cream, a stick, a spray, an aerosol, foam, a paste, a mousse and a variety of cosmetics or skin-care formulations including solid, semi-solid, or a liquid make-up such as foundations, eye make-up, etc.

In some embodiments the liquid may be applied onto the skin as a moisturizer.

In some embodiments, the composition of the invention is formulated as a lotion.

In some embodiments, the composition of the invention is formulated as an emulsion.

In some embodiments, the composition of the invention is formulated as a facial formulation.

In some embodiments, the composition of the invention is formulated as a body formulation.

In some embodiments, the composition of the invention is formulated as a leave on formulation.

In some embodiments, the composition of the invention is formulated as rinse off formulation.

As used herein, a “leave on” (in contrary to “rinse off”) composition/formulation refers to a composition/formulation that may be in prolonged contact with the skin and can be applied to a skin region without the need to remove it from the skin (e.g., by wiping or rinsing it off) in any way.

In some embodiments, the leave-on composition/formulation may be adapted to be applied to a skin region and to be left on the skin for a time sufficient to achieve an end result.

The viscosity of the composition according to the invention may vary depending on the form (i.e., lotion, cream, etc.), concentration of the active combination, the carrier, the purpose (i.e., cosmetic or therapeutic), end user and other parameters.

The compositions according to the invention (cosmetic or therapeutic) may comprise at least one dermatological, cosmetically or pharmaceutically acceptable additive selected amongst inert and effect-inducing additives. In some embodiments, the inert additive is selected from a diluent, a preservative, an abrasive, an anti-caking agent, an antistatic agent, a binder, a buffer, a dispersant, an emollient, an emulsifier, a co-emulsifiers, a fibrous material, a film forming agent, a fixative, a foaming agent, a foam stabilizer, a foam booster, a gallant, a lubricant, a moisture barrier agent, an opacifier (e.g., styrene/acrylamide copolymer), a plasticizer, a preservative, a propellant, a stabilizer, a surfactant, a suspending agent, a thickener, a wetting agent, and a liquefier.

In some embodiments, the at least one inert additive is a smoothness enhancer ingredient, such as silica.

In some embodiments, each of the at least one dermatological, cosmetically or pharmaceutically acceptable additives may constitute between about 0.05% to 15% of the total weight of the formulation. In some embodiments, the at least one additive constitutes between 0.05% and 10% or between 0.05% and 8%, or between 0.05% and 7%, or between 0.05% and 6%, or between 0.05% and 5% of the total weight of the formulation.

In some embodiments, the at least one inert additive is a diluent being selected from water, Bisabolol, propane diol, propylene glycol, butylene glycol, glycerin, safflower oil and mixtures thereof.

In some embodiments, the at least one inert additive is a preservative being selected from one or more of methylparaben, methyldibromo glutaronitrile, phenethyl alcohol, glyceryl caprilate, propylparaben, methylisothiazolinone, decylene glycol, dehydroacetic acid, phenoxyethanol, benzoic acid, 2-methyl-2H-isothiazoline-3-one, polyethylene glycol monococoate, polyethylene glycol dicocoate, polyethylene Glycol, iodopropynyl butylcarbamate, 1.2-hexanediol, caprylyl glycol, imidazolidinyl urea, DMDM Hydantoin, Ipbc, MIT, 2,3-bronopol.

In further embodiments, the inert additive is an emulsifier being selected from one or more of cetyl hydroxyethylcellulose, cetyl alcohol, ceteth-20 (a polyethylene glycol derivative of cetyl alcohol), cetearyl olivate, cetyl palmitate, sorbitan olivate, sorbitan palmitate, stearates, steareth-20 (polyethylene glycol ethers of stearic acid-octadecyl polyoxyethylene ether), steareth-25.

In some embodiments, the stearate is selected from PEG-40 stearate, glyceryl steatrate, sorbitan tristearate, stearyl alcohol and mixtures thereof.

In some embodiments, the stearate is glyceryl stearate.

In still other embodiments, the inert additive is an emollient, being selected from vegetable and animal fats and oils such as castor oil, hydrogenated castor oil, cocoa butter, safflower oil, cottonseed oil, corn oil, olive oil, cod liver oil, almond oil, avocado oil, palm oil, sesame oil, squalene, phytosqalene, kikui oil, chamomilla recutita (matricaria) flower oil, hypericum perforatum oil, soybean oil and Vitis vinifera (grape) seed oil; acetoglyceride esters, such as acetylated monoglycerides; alkyl esters of fatty acids having 10 to 24 carbon atoms which include, but are not limited to, methyl, isopropyl, and butyl esters of fatty acids such as hexyl laurate, isohexyl laurate, ethylhexyl palmitate, isohexyl palmitate, isopropyl palmitate, octyl palmitate, decyloleate, isodecyl oleate, hexadecyl stearate decyl stearate, isopropyl isostearate, diisopropyl adipate, diisohexyl adipate, dihexyldecyl adipate, diisopropyl sebacate, lauryl lactate, myristyl lactate, and cetyl lactate; alkenyl esters of fatty acids having 10 to 20 carbon atoms such as oleyl myristate, oleyl stearate, and oleyl oleate; fatty acids having 10 to 20 carbon atoms such as pelargonic, lauric, myristic, palmitic, stearic, isostearic, hydroxystearic, oleic, linoleic, ricinoleic, arachidic, behenic, and erucic acids; fatty alcohols having 10 to 20 carbon atoms such as lauryl, myristyl, cetyl, hexadecyl, stearyl, isostearyl, hydroxystearyl, oleyl, ricinoleyl, behenyl, erucyl, and 2-octyl dodecanyl alcohols; fatty alcohol ethers such as propoxylated fatty alcohols of 10 to 20 carbon atoms which include, but are not limited to, lauryl, cetyl, stearyl, isostearyl, oleyl, and cholesterol alcohols, having attached thereto from 1 to 50 propylene oxide groups; lanolin and lanolin derivatives such as lanolin, lanolin oil, lanolin wax, lanolin alcohols, lanolin fatty acids, isopropyl lanolate, ethoxylated lanolin, ethoxylated lanolin alcohols, ethoxylated cholesterol, propoxylated lanolin alcohols, acetylated lanolin alcohols, lanolin alcohols linoleate, lanolin alcohols ricinoleate, acetate of lanolin alcohols ricinoleate, acetate of ethoxylated alcohols-esters, bydrogenolysis of lanolin, ethoxylated sorbitol lanolin, and liquid and semisolid lanolin absorption bases; polyhydric alcohol esters such as ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, glyceryl mono- and di-fatty acid esters, polyglycerol polyfatty esters, ethoxylated glyceryl monostearate, 1,2-butylene glycol monostearate, 1,2-butylene glycol distearate, polyoxyethylene polyol fatty acid ester, sorbitan fatty acid esters, and polyoxyethylene sorbitan fatty acid esters; Wax esters such as beeswax, spermaceti, myristyl myristate, stearyl stearate; forming a mixture of ether esters; vegetable waxes including, but not limited to, carnauba and candelilla waxes; surface active silicone derivatives such as cyclopentasiloxane PEG/PPG-18/18 dimethicone, dimethicone, dimethicone crosspolymer, cyclomethicone, cyclomethicone&dimethiconol; caprylic/capric triglyceride; and cholesterol fatty acid esters and any mixtures thereof.

In some embodiments, each of the at least one inert additive may constitute between about 0.05% to 15% of the total weight of the formulation. In some embodiments, the at least one inert additive constitutes between 0.05% and 10% or between 0.05% and 8%, or between 0.05% and 7%, or between 0.05% and 6%, or between 0.05% and 5% of the total weight of the formulation.

In other embodiments, the effect-inducing additive is selected from an anti-acne agent, an anti-aging agent, an antibacterial agent, an anti-cellulites agent, an antidandruff agent, an antifungal agent, an anti-inflammatory agent, an anti-irritation agent (e.g., allantoin, Aloe Barbadensis leaf juice), an antimicrobial agent, an antioxidant (e.g., butylated hydroxyanisole, propyl gallate, an antiperspirant agent, an antiseptic agent, a cell stimulant, a cleansing agent, a conditioner, a deodorant, a fragrance ingredient (e.g., perfume, limonene), a depilatory, a detergent, an enzyme, an essential oil, an exfoliant, a fungicide, a glosser, hair conditioner (hair conditioner agent), hair set resin, hair sheen agent, hair waving agent, a humectants (e.g., Erythritol, Homarine HCl, Ceratonia Siliqua (carob bean) gum), a moisturizer (e.g., sodium hyaluronate), an ointment base, a perfume, a protein, a skin calming agent, a skin cleanser, a skin conditioner (skin conditioning agent), a skin healing agent, a skin lightening agent, a skin protectant, a skin smoothing agent, a skin softening agent, a skin soothing agent, a sunscreen agent, a tanning accelerator, vitamins, a colorant, and a flavoring agent.

In some embodiments, the at least one additive is a sunscreen, such as Ethyl hexyl methoxycinnamate or titanium dioxide.

In some embodiments, each of the at least one effect-inducing additive may constitute between about 0.05% to 15% of the total weight of the formulation. In some embodiments, the at least one inert additive constitutes between 0.05% and 10% or between 0.05% and 8%, or between 0.05% and 7%, or between 0.05% and 6%, or between 0.05% and 5% of the total weight of the formulation.

The cosmetic or pharmaceutical compositions of the invention may also comprise pharmaceutical actives useful in the form of a chemical substance, material or compound, e.g., suitable for topical administration, to induce a desired local or systemic effect. Non-limiting examples of such actives are an antibiotic, an antiviral agent, an analgesic (e.g. ibuprofen, acetyl salicylic acid, naproxen, and the like), an antihistamine, an anti-inflammatory agent, an antipruritic, an antipyretic, an anesthetic agent, a diagnostic agent, a hormone, an antifungal agent, an antimicrobial agent, a cutaneous growth enhancer, a pigment modulator, an antiproliferative, an antipsoriatic, a retinoid, an anti-acne medicament (e.g. benzoyl peroxide, sulfur, and the like), an antineoplastic agent, a phototherapeutic agent, a keratolys (e.g. resorcinol, salicylic acid, and the like) and mixtures thereof.

Application of a composition of the invention onto the skin of a subject, for cosmetic/skin-care or therapeutic purposes may be in a single dose, in multiple doses, in a continuous or intermittent manner, depending, for example, upon the subject's physiological condition, whether the purpose of the administration is cosmetic or therapeutic/prophylactic and other factors known to the medical practitioner. The application of a composition of the invention may be essentially continuous over a pre-selected period of time or may be in a series of spaced doses.

The compositions of the invention are typically prepared by combining the ingredients of the active combination in appropriate concentrations. Other active or inert additives selected by one of skill in the art may optionally be added. The absolute weight of a given active agent included in a unit dose can vary widely. For example, about 0.1 microgram to about 5 g, or about 1 microgram to about 1 g, or about 10 micrograms to about 500 mg, of at least one of the components can be administered by topical administration.

The compositions of the invention, being substantially for topical use, may be a skin-care formulation or a therapeutic formulation.

In some embodiments, the compositions of the invention are skin-care or dermo-pharmaceutical compositions (including, e.g., toiletries, health and beauty aids and cosmeceuticals) used for cosmetic and personal skin-care applications.

The term “cosmetic composition” or “skin care composition” relates to a composition of the invention that can be used for cosmetic purposes, purposes of hygiene or skin-care or as a basis for delivery of one or more pharmaceutical ingredients. It is also possible that these compositions are used for two or more of these same purposes at one time. For example, a medicated dandruff shampoo may be used as a personal care product, i.e., to provide clean hair, and at the same time have pharmacological properties.

In some embodiments, the cosmetic compositions are for promoting bodily attractiveness, cover or mask the physical manifestations of a disorder or disease, modulate or alleviate wrinkling, unevenness and dryness in the skin of a mammal. The compositions additionally regulate skin condition and signs of skin aging (all perceptible manifestations as well as any other macro or micro effects) by regulating visible and/or tactile discontinuities in skin texture, including fine lines, wrinkles, enlarged pores, roughness and other skin texture discontinuities associated with aged skin with reduced irritation and dryness.

Thus, according to another one of its aspect the present invention provides a composition (formulation) according to the invention for one or more of protecting and/or improving the state of the skin, and preventing and/or treating imperfections of the skin of a subject in need thereof.

According to another one of its aspect the present invention provides a composition (formulation) according to the invention for use in a method of one or more of protecting and/or improving the state of the skin, and preventing and/or treating imperfections of the skin of a subject in need thereof.

According to a further one of its aspect the present invention provides a method of one or more of protecting and/or improving the state of the skin, and preventing and/or treating imperfections of the skin of a subject in need thereof, said method comprising topically administering a composition according to the invention onto the skin of said subject.

In some embodiments, the method/composition of the invention is used for treating rings under the eye, symptoms of aging, protecting the skin, increasing the detoxification of xenobiotics, intervening on pigmentation level, inhibiting melanogenesis, stimulating the detoxification systems, stimulating hair and body hair growth, modulating DHT levels, intervening on adipocytes, and promoting lipolysis.

In some embodiments, the method/composition of the invention is used for rejuvenating the skin.

In some embodiments the state of the skin may be associated with and/or mediated by and/or affected by and/or related to one or more of biological pathways being selected from cell cycle (KEGG); signaling pathways regulating pluripotency of stem cells (KEGG); nucleotide excision repair (KEGG); P53 signaling pathway (KEGG); assembly of collagen fibrils and other multimeric structures (WIKI); Elastic fiber formation (WIKI); keratinization (WIKI); or any combination thereof.

In some embodiments the state of the skin may be associated with and/or mediated by and/or affected by and/or related to one or more of biological pathways being selected from sphingolipid metabolism (KEGG); NF KAPP B signaling pathway (KEGG); TNT signaling pathway (KEGG); apoptosis (KEGG); base excision repair (KEGG); nod-like receptor signaling pathway (KEGG); Yersinia infection (KEGG); keratinization (WIKI) or any combination thereof.

In some embodiments the state of the skin may be associated with and/or mediated by and/or affected by and/or related to one or more of biological pathways being selected from cell cycle (KEGG); signaling pathways regulating pluripotency of stem cells (KEGG); nucleotide excision repair (KEGG); P53 signaling pathway (KEGG); assembly of collagen fibrils and other multimeric structures (WIKI); Elastic fiber formation (WIKI); keratinization (WIKI); sphingolipid metabolism (KEGG); NF KAPP B signaling pathway (KEGG); TNT signaling pathway (KEGG); apoptosis (KEGG); base excision repair (KEGG); nod-like receptor signaling pathway (KEGG); Yersinia infection (KEGG); or any combination thereof.

In some embodiments, the method is associate with non-medical condition of the skin.

In some embodiments, the method is associate with a medical condition of the skin.

In some embodiments, the method is for protecting and/or improving the state of the skin.

In some embodiments, the method is for preventing and/or treating imperfections of the skin of a subject.

In some embodiments, the compositions of the invention are for use in a method for protecting and/or improving the state of the skin.

In some embodiments, the compositions of the invention are for use in a method for preventing and/or treating imperfections of the skin of a subject.

In some embodiments the protecting and/or improving the state of the skin activity of the compositions of the present invention and/or the preventing and/or treating imperfections of the skin of a subject activity of the compositions of the present invention may be mediated by retinol.

In some embodiments the imperfections of the skin of a subject may be due to deficiency of retinol.

In other embodiments, the compositions are pharmaceutical composition used in the treatment or prevention of at least one disease or disorder (e.g., of the skin).

In some embodiments the treatment or prevention of at least one disease or disorder may be mediated by retinol.

In another aspect of the present invention, there is provided a use of at least one Dead Sea extract, β-carotene and Niacinamide for the preparation of a composition/formulation.

In another aspect of the present invention, there is provided a use of at least one Dead Sea extract, at least one extract of Dunaliella alga selected from one or both of Dunaliella Salina extract and Dunaliella Bardawill extract, β-carotene and Niacinamide for the preparation of a composition/formulation.

The compositions of the invention, in some embodiments, are formulated for use in the treatment of a disease or disorder.

Thus, the present invention also provides a method of therapeutic treatment or prophylaxis of such disease or disorder.

In some embodiments the disease or disorder is skin related.

In a further aspect, there is provided a method for treating a disease or disorder of the skin, the method comprising administering to a subject in need thereof a composition according to the invention.

In some embodiments the administration is topical administration.

In some embodiments, the subject is suffering, or has predisposition to suffer, or is one which may be exposed to conditions which increase the chances of suffering from a disease or disorder of the skin, which is optionally (may or may not be) related to one or more of age, sex, skin color, skin wounds, exposure to the sun, UV radiation, inflammation, irritation, a pre-existence of a disease not associated with the skin, etc.

In some embodiments the treatment or prevention of at least one disease or disorder may be mediated by one or more Vitamin A e.g., retinol and/or by one or more precursors of Vitamin A.

In some embodiments, the disease or disorder of the skin may relate to deficiency of one or more Vitamin A e.g., retinol and/or one or more Vitamin A precursor.

In some embodiments, the disease or disorder of the skin is related to sun exposure.

In some embodiments, the disease or disorder of the skin is a secondary condition, e.g., inflammation, being related to an existing condition.

In some embodiments, the disease or disorder of the skin is skin irritation which may be related to an existing condition.

In further embodiments, the disease or disorder of the skin are age-related.

Non-limiting examples of such diseases or disorders of the skin are wounds, acne, psoriasis, atopic skin, diabetic skin, dermatitis, eczema, xerotic, dry skin, and chaffed skin.

In some embodiments, the administration of the composition according to the invention is topical.

In a further one of its aspect the present invention provides compositions according to the invention for use in the treatment and/or prevention of one or more disease or disorder, the disease or disorder being associated with and/or being mediated by and/or being affected by and/or being related to one or more of biological pathways being selected from cell cycle (KEGG); signaling pathways regulating pluripotency of stem cells (KEGG); nucleotide excision repair (KEGG); P53 signaling pathway (KEGG); assembly of collagen fibrils and other multimeric structures (WIKI); Elastic fiber formation (WIKI); keratinization (WIKI); or any combination thereof.

In a further one of its aspect the present invention provides compositions according to the invention for use in the treatment and/or prevention of one or more disease or disorder, the disease or disorder being associated with and/or being mediated by and/or being affected by and/or being related to one or more of biological pathways being selected sphingolipid metabolism (KEGG); NF KAPP B signaling pathway (KEGG); TNT signaling pathway (KEGG); apoptosis (KEGG); base excision repair (KEGG); nod-like receptor signaling pathway (KEGG); Yersinia infection (KEGG); keratinization (WIKI) or any combination thereof.

In a further one of its aspect the present invention provides compositions according to the invention for use in the treatment and/or prevention of one or more disease or disorder, the disease or disorder being associated with and/or being mediated by and/or being affected by and/or being related to one or more of biological pathways being selected from cell cycle (KEGG); signaling pathways regulating pluripotency of stem cells (KEGG); nucleotide excision repair (KEGG); P53 signaling pathway (KEGG); assembly of collagen fibrils and other multimeric structures (WIKI); Elastic fiber formation (WIKI); keratinization (WIKI); sphingolipid metabolism (KEGG); NF KAPP B signaling pathway (KEGG); TNT signaling pathway (KEGG); apoptosis (KEGG); base excision repair (KEGG); nod-like receptor signaling pathway (KEGG); Yersinia infection (KEGG); or any combination thereof.

In some embodiments the disease or disorder may be associated with and/or mediated by and/or affected by and/or related to cell cycle (KEGG) pathway.

Regarding cell cycle (KEGG) pathway, mitotic cell cycle progression is accomplished through a reproducible sequence of events, DNA replication (S phase) and mitosis (M phase) separated temporally by gaps known as G1 and G2 phases. Cyclin-dependent kinases (CDKs) are key regulatory enzymes, each consisting of a catalytic CDK subunit and an activating cyclin subunit. CDKs regulate the cell's progression through the phases of the cell cycle by modulating the activity of key substrates. Downstream targets of CDKs include transcription factor E2F and its regulator Rb. Precise activation and inactivation of CDKs at specific points in the cell cycle are required for orderly cell division. Cyclin-CDK inhibitors (CKIs), such as p16Ink4a, p15Ink4b, p27Kip1, and p21Cip1, are involved in the negative regulation of CDK activities, thus providing a pathway through which the cell cycle is negatively regulated. Eukaryotic cells respond to DNA damage by activating signaling pathways that promote cell cycle arrest and DNA repair. In response to DNA damage, the checkpoint kinase ATM phosphorylates and activates Chk2, which in turn directly phosphorylates and activates p53 tumor suppressor protein. p53 and its transcriptional targets play an important role in both G1 and G2 checkpoints. ATR-Chk1-mediated protein degradation of Cdc25A protein phosphatase is also a mechanism conferring intra-S-phase checkpoint activation. At least one of the above may be affected by the compositions of the present invention.

In some embodiments the composition of the present invention may improve the skin conditions via affecting the cell cycle (KEGG) pathway. At times, the effect may promote skin proliferation and renewal.

In some embodiments said disease or disorder may be associated with and/or mediated by and/or affected by and/or related to signaling pathways regulating pluripotency of stem cells (KEGG) pathway.

Regarding signaling pathways regulating pluripotency of stem cells (KEGG) pathway, pluripotent stem cells (PSCs) are basic cells with an indefinite self-renewal capacity and the potential to generate all the cell types of the three germinal layers. The types of PSCs known to date include embryonic stem (ES) and induced pluripotent stem (iPS) cells. ES cells are derived from the inner cell mass (ICM) of blastocyst-stage embryos. iPS cells are generated by reprogramming somatic cells back to pluripotent state with defined reprogramming factors, Oct4, Sox2, Klf4 and c-Myc (also known as Yamanaka factors). PSCs including ES cells and iPS cells are categorized into two groups by their morphology, gene expression profile and external signal dependence. Conventional mouse-type ES/iPS cells are called ‘naive state’ cells. They are mainly maintained under the control of LIF and BMP signaling. On the other hand, human-type ES/iPS cells, which are in need of Activin and FGF signaling, are termed ‘primed state’. However, these signaling pathways converge towards the activation of a core transcriptional network that is similar in both groups and involves OCt4, Nanog and Sox2. The three transcription factors and their downstream target genes coordinately promote self-renewal and pluripotency. At least one of the above may be affected by the compositions of the present invention.

In some embodiments the composition of the present invention may improve the skin conditions via affecting the signaling pathways regulating pluripotency of stem cells (KEGG) pathway. At times, the effect may promote skin differentiation and renewal.

In some embodiments said disease or disorder may be associated with and/or mediated by and/or affected by and/or related to nucleotide excision repair (KEGG) pathway.

Regarding nucleotide excision repair (NER) (KEGG) pathway, NER is a mechanism to recognize and repair bulky DNA damage caused by compounds, environmental carcinogens, and exposure to UV-light. In humans hereditary defects in the NER pathway are linked to at least three diseases: xeroderma pigmentosum (XP), Cockayne syndrome (CS), and trichothiodystrophy (TTD). The repair of damaged DNA involves at least 30 polypeptides within two different sub-pathways of NER known as transcription-coupled repair (TCR-NER) and global genome repair (GGR-NER). TCR refers to the expedited repair of lesions located in the actively transcribed strand of genes by RNA polymerase II (RNAP II). In GGR-NER the first step of damage recognition involves XPC-hHR23B complex together with XPE complex (in prokaryotes, uvrAB complex). The following steps of GGR-NER and TCR-NER are similar. At least one of the above may be affected by the compositions of the present invention.

In some embodiments the composition of the present invention may improve the skin conditions via affecting the nucleotide excision repair (KEGG) pathway. At times, the effect may be protection of the skin from UV damage.

In some embodiments the disease is selected from one or more of xeroderma pigmentosum (XP), Cockayne syndrome (CS) and trichothiodystrophy (TTD).

In some embodiments said disease or disorder may be associated with and/or mediated by and/or affected by and/or related to P53 signaling pathway (KEGG).

Regarding P53 signaling pathway (KEGG), p53 activation is induced by a number of stress signals, including DNA damage, oxidative stress and activated oncogenes. The p53 protein is employed as a transcriptional activator of p53-regulated genes. This results in three major outputs; cell cycle arrest, cellular senescence or apoptosis. Other p53-regulated gene functions communicate with adjacent cells, repair the damaged DNA or set up positive and negative feedback loops that enhance or attenuate the functions of the p53 protein and integrate these stress responses with other signal transduction pathways. At least one of the above may be affected by the compositions of the present invention.

In some embodiments the composition of the present invention may improve the skin conditions via affecting the P53 signaling pathway (KEGG). At times, the effect may be skin protection against stress.

In some embodiments the disease or disorder may be associated with and/or mediated by and/or affected by and/or related to assembly of collagen fibrils and other multimeric structures (WIKI) pathway.

Regarding assembly of collagen fibrils and other multimeric structures (WIKI) pathway, collagen trimers in triple-helical form, referred to as procollagen or collagen molecules, are exported from the ER and trafficked through the Golgi network before secretion into the extracellular space. For fibrillar collagens namely types I, II, III, V, XI, XXIV and XXVII secretion is concomitant with processing of the N and C terminal collagen propeptides. These processed molecules are known as tropocollagens, considered to be the units of higher order collagen structures. They form within the extracellular space via a process that can proceed spontaneously, but in the cellular environment is regulated by many collagen binding family collagens and Small Leucine-Rich Proteoglycans (SLRPs). At least one of the above may be affected by the compositions of the present invention.

In some embodiments the composition of the present invention may improve the skin conditions via affecting the assembly of collagen fibrils and other multimeric structures (WIKI) pathway. At times, the effect may be related to epidermal dermal structure and connection and to skin elasticity.

In some embodiments the disease or disorder may be associated with and/or mediated by and/or affected by and/or related to Elastic fiber formation (WIKI) pathway.

Regarding elastic fiber formation (WIKI) pathway, elastic fibres (EF) are a major structural constituent of dynamic connective tissues such as large arteries and lung parenchyma, where they provide essential properties of elastic recoil and resilience. EF are composed of a central cross-linked core of elastin, surrounded by a mesh of microfibrils, which are composed largely of fibrillin. In addition to elastin and fibrillin-1, over 30 ancillary proteins are involved in mediating important roles in elastic fibre assembly as well as interactions with the surrounding environment. These include fibulins, elastin microfibril interface located proteins (EMILINs), microfibril-associated glycoproteins (MAGPs) and Latent TGF-beta binding proteins (LTBPs). At least one of the above may be affected by the compositions of the present invention.

In some embodiments the composition of the present invention may improve the skin conditions via affecting the elastic fiber formation (WIKI) pathway. At times, the effect may be on skin firmness and elasticity.

In some embodiments the disease or disorder may be associated with and/or mediated by and/or affected by and/or related to keratinization (WIKI) pathway.

Regarding keratinization (WIKI) pathway, keratins are the major structural protein of vertebrate epidermis, constituting up to 85% of a fully differentiated keratinocyte. Keratins belong to a superfamily of intermediate filament (IF) proteins that form alpha-helical coiled-coil dimers, which associate laterally and end-to-end to form approximately 10 nm diameter filaments. Keratin filaments are heteropolymeric, formed from equal amounts of acidic type I and basic/neutral type 2 keratins. Humans have 54 keratin genes. They have highly specific expression patterns, related to the epithelial type and stage of differentiation. Roughly half of human keratins are specific to hair follicles. Keratin filaments bundle into tonofilaments that span the cytoplasm and bind to desmosomes and other cell membrane structures. This reflects their primary function, maintaining the mechanical stability of individual cells and epithelial tissues. At least one of the above may be affected by the compositions of the present invention.

In some embodiments the composition of the present invention may improve the skin conditions via affecting the keratinization (WIKI) pathway. At times, the effect may be efficient epidermis differentiation and renewal.

In some embodiments the disease or disorder may be associated with and/or mediated by and/or affected by and/or related to sphingolipid metabolism (KEGG) pathway.

Regarding sphingolipid metabolism (KEGG) pathway, mammalian epidermis produces and delivers large quantities of glucosylceramide and sphingomyelin precursors to stratum corneum extracellular domains, where they are hydrolyzed to corresponding ceramide species. This cycle of lipid precursor formation and subsequent hydrolysis represents a mechanism that protects the epidermis against potentially harmful effects of ceramide accumulation within nucleated cell layers. Prominent skin disorders, such as psoriasis and atopic dermatitis, have diminished epidermal ceramide levels, reflecting altered sphingolipid metabolism, that may contribute to disease severity/progression. At least one of the above may be affected by the compositions of the present invention.

In some embodiments the composition of the present invention may improve the skin conditions via affecting the sphingolipid metabolism (KEGG) pathway.

In some embodiments the disease or disorder may be associated with and/or mediated by and/or affected by and/or related to NF KAPP B signaling pathway (KEGG).

Regarding NF KAPP B signaling pathway (KEGG), Nuclear factor-kappa B (NF-kappa B) is the generic name of a family of transcription factors that function as dimers and regulate genes involved in immunity, inflammation and cell survival. There are several pathways leading to NF-kappa B-activation. The canonical pathway is induced by tumor necrosis factor-alpha (TNF-alpha), interleukin-1 (IL-1) or byproducts of bacterial and viral infections. This pathway relies on IKK-mediated IkappaB-alpha phosphorylation on Ser32 and 36, leading to its degradation, which allows the p50/p65 NF-kappa B dimer to enter the nucleus and activate gene transcription. Atypical pathways are IKK-independent and rely on phosphorylation of IkappaB-alpha on Tyr42 or on Ser residues in IkappaB-alpha PEST domain. The non-canonical pathway is triggered by particular members of the TNFR superfamily, such as lymphotoxin-beta (LT-beta) or BAFF. It involves NIK and IKK-alpha-mediated p100 phosphorylation and processing to p52, resulting in nuclear translocation of p52/RelB heterodimers.

At least one of the above may be affected by the compositions of the present invention.

In some embodiments the composition of the present invention may improve the skin conditions via affecting the NF KAPP B signaling pathway (KEGG).

In some embodiments the disease or disorder may be associated with and/or mediated by and/or affected by and/or related to KEGG_TNF_SIGNALING_PATHWAY.

Regarding KEGG_TNF_SIGNALING_PATHWAY, Tumor necrosis factor (TNF), as a critical cytokine, can induce a wide range of intracellular signal pathways including apoptosis and cell survival as well as inflammation and immunity. Activated TNF is assembled to a homotrimer and binds to its receptors (TNFR1, TNFR2) resulting in the trimerization of TNFR1 or TNFR2. TNFR1 is expressed by nearly all cells and is the major receptor for TNF (also called TNF-alpha). In contrast, TNFR2 is expressed in limited cells such as CD4 and CD8 T lymphocytes, endothelial cells, microglia, oligodendrocytes, neuron subtypes, cardiac myocytes, thymocytes and human mesenchymal stem cells. It is the receptor for both TNF and LTA (also called TNF-beta). Upon binding of the ligand, TNFR mediates the association of some adaptor proteins such as TRADD or TRAF2, which in turn initiate recruitment of signal transducers. TNFR1 signaling induces activation of many genes, primarily controlled by two distinct pathways, NF-kappa B pathway and the MAPK cascade, or apoptosis and necroptosis. TNFR2 signaling activates NF-kappa B pathway including PI3K-dependent NF-kappa B pathway and JNK pathway leading to survival.

At least one of the above may be affected by the compositions of the present invention.

In some embodiments the composition of the present invention may improve the skin conditions via affecting the KEGG_TNF_SIGNALING_PATHWAY.

In some embodiments the disease or disorder may be associated with and/or mediated by and/or affected by and/or related to KEGG_APOPTOSIS pathway.

Regarding KEGG_APOPTOSIS pathway, apoptosis is a genetically programmed process for the elimination of damaged or redundant cells by activation of caspases (aspartate-specific cysteine proteases). The onset of apoptosis is controlled by numerous interrelating processes. The ‘extrinsic’ pathway involves stimulation of members of the tumor necrosis factor (TNF) receptor subfamily, such as TNFRI, CD95/Fas or TRAILR (death receptors), located at the cell surface, by their specific ligands, such as TNF-alpha, FasL or TRAIL, respectively. The ‘intrinsic’ pathway is activated mainly by non-receptor stimuli, such as DNA damage, ER stress, metabolic stress, UV radiation or growth-factor deprivation. The central event in the ‘intrinsic’ pathway is the mitochondrial outer membrane permeabilization (MOMP), which leads to the release of cytochrome c. These two pathways converge at the level of effector caspases, such as caspase-3 and caspase-7. The third major pathway is initiated by the constituents of cytotoxic granules (e.g. Perform and Granzyme B) that are released by CTLs (cytotoxic T-cells) and NK (natural killer) cells. Granzyme B, similarly to the caspases, cleaves its substrates after aspartic acid residues, suggesting that this protease has the ability to activate members of the caspase family directly. It is the balance between the pro-apoptotic and anti-apoptotic signals that eventually determines whether cells will undergo apoptosis, survive or proliferate. TNF family of ligands activates anti-apoptotic or cell-survival signals as well as apoptotic signals. NGF and Interleukin-3 promotes the survival, proliferation and differentiation of neurons or hematopoietic cells, respectively. Withdrawal of these growth factors leads to cell death.

At least one of the above may be affected by the compositions of the present invention.

In some embodiments the composition of the present invention may improve the skin conditions via affecting the KEGG_APOPTOSIS pathway.

In some embodiments the disease or disorder may be associated with and/or mediated by and/or affected by and/or related to KEGG_BASE_EXCISION_REPAIR pathway.

Regarding KEGG_BASE_EXCISION_REPAIR pathway, base excision repair (BER) is the predominant DNA damage repair pathway for the processing of small base lesions, derived from oxidation and alkylation damages. BER is normally defined as DNA repair initiated by lesion-specific DNA glycosylases and completed by either of the two sub-pathways: short-patch BER where only one nucleotide is replaced and long-patch BER where 2-13 nucleotides are replaced. Each sub-pathway of BER relies on the formation of protein complexes that assemble at the site of the DNA lesion and facilitate repair in a coordinated fashion. This process of complex formation appears to provide an increase in specificity and efficiency to the BER pathway, thereby facilitating the maintenance of genome integrity by preventing the accumulation of highly toxic repair intermediates.

At least one of the above may be affected by the compositions of the present invention.

In some embodiments the composition of the present invention may improve the skin conditions via affecting the KEGG_BASE_EXCISION_REPAIR pathway.

In some embodiments the disease or disorder may be associated with and/or mediated by and/or affected by and/or related to KEGG_NOD-LIKE_RECEPTOR SIGNALING pathway.

In some embodiments the disease or disorder may be associated with and/or mediated by and/or affected by and/or related to KEGG_YERSINIA_INFECTION pathway.

Regarding KEGG_NOD-LIKE_RECEPTOR SIGNALING pathway and KEGG_YERSINIA_INFECTION pathway, NLRP3 Inflammasome signaling pathway is a subset of NOD-like receptor signaling pathway. Yersinia YopM to NLRP3 Inflammasome signaling pathway is a subset of Yersinia infection. The process of aging is associated with the appearance of low-grade subclinical inflammation, termed inflammaging, that can accelerate age-related diseases. Inflammasome activation has been directly linked to the process of inflammaging. It has been demonstrated that in aging human fibroblasts, inflammasomes accumulate and that control of inflammasome activation can help improve cell survivability.

At least one of the above may be affected by the compositions of the present invention.

In some embodiments the composition of the present invention may improve the skin conditions via affecting the Regarding KEGG_NOD-LIKE_RECEPTOR SIGNALING pathway. At times, the effect may be inflammasome related.

In some embodiments the composition of the present invention may improve the skin conditions via affecting the KEGG_YERSINIA_INFECTION pathway. At times, the effect may be inflammasome related.

In some embodiments the compositions of the present invention may beneficially affect the skin via one or more of promoting skin proliferation and renewal, promoting skin differentiation, protecting the skin from UV damage, protecting the skin against stress.

In some embodiments the composition of the present invention may improve the skin conditions via affecting the synthesis in fibroblasts of Collagen I, as such beneficially affecting dermal connective tissue.

In some embodiments the disease or disorder may be associated with deficiency of one or more Vitamin A e.g., retinol and/or by one or more precursors of Vitamin A.

In some embodiments in the method/s disclosed herein the administrations is topical administration onto (at least a region) the skin of the subject.

In some embodiments the compositions of the present invention affect one or more of the aforementioned pathways via increase in one or more Vitamin A e.g., retinol production e.g., as a result of topical application onto the skin.

In some embodiments the compositions of the present invention beneficially affect the skin via affecting one or more cellular biological mechanisms (e.g., by reducing the damage of the cellular natural processes).

In some embodiments the compositions of the present invention beneficially affect the skin via coping with stress.

In some embodiments the compositions of the present invention beneficially affect the skin via optimizing cellular metabolic balance and regeneration.

In some embodiments the compositions of the present invention beneficially affect the skin via affecting one or more gene-expression and/or one or more protein expression.

In some embodiments the compositions of the present invention beneficially affect the skin at the molecular level e.g., by affecting (e.g., enhancing or reducing) the expression of one or more molecules that are involved in skin related conditions.

In some embodiments the compositions of the present invention improve fine wrinkles and affected markers of photo-aging, including matrix metalloproteinase, collagenase, and collagen.

In some embodiments the compositions of the present invention beneficially encourage keratinocyte proliferation.

The term “topical” as used herein above and below refers to the application of a composition according to the invention directly onto at least a portion/region of a subject's skin (human's or non-human's skin) so as to achieve a desired effect, e.g., cosmetic or therapeutic effect, at the site of application. In some embodiments, the desired effect is achieved at the site of application without inducing one or more systemic effects. In other embodiments, the formulation of the invention induces at least a partial systemic effect which contributes to the induction of at least one desired effect.

The term “skin” as used herein above and below refers to any part of the human or animal skin, including the whole surface thereof, hair and nails.

The term “treatment” as used herein above and below refers to administration (e.g., topical) of an effective amount of a composition of the present invention effective to ameliorate undesired symptoms associated with a disease/disorder (e.g., skin disease), to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease form occurring or a combination of two or more of the above.

In some embodiments the disease and/or disorder is a non-medical condition e.g., associated with normal skin conditions.

In some embodiments the disease and/or disorder is a medical condition e.g., associate with pathological skin conditions.

The “effective amount”, whether a therapeutically or cosmetically effective amount for purposes herein, is determined by such considerations as may be known in the art. The amount must be effective to achieve one or more of the above desired therapeutic or cosmetic effects, depending, inter alia, on the type and severity of the disease to be treated and the treatment regime. The effective amount is typically determined in appropriately designed clinical trials (dose range studies) and the person versed in the art will know how to properly conduct such trials in order to determine the effective amount. As generally known, an effective amount depends on a variety of factors including the affinity of the ligand to the receptor, its distribution profile, a variety of pharmacological parameters such as half-life on the skin, on undesired side effects, if any, on factors such as age and gender, etc.

Unless otherwise noted, the concentrations of the various ingredients in the compositions/formulations disclosed herein are provided in weight per weight ratio (w/w) i.e., the weight in grams of the ingredient per 100 grams total weight of the composition/formulation.

As used herein above and below the term “about” refers to ±10% of the indicated value.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

As used herein, the singular form “a”, “an” and “the” include plural references unless the context clearly dictates otherwise. For example, the term “an extract” or “at least one extract” may independently include a plurality of extracts, including a variety thereof.

It is noted that various embodiments detailed herein above in connection with a specific aspect may be applicable to one or more other aspects of the invention.

Various embodiments and aspects of the present invention as delineated hereinabove and as claimed in the claims section below find experimental support in the following examples.

### DETAILED DESCRIPTION OF EMBODIMENTS

The following examples are not in any way intended to limit the scope of the invention as claimed.

### Example 1: Dead Sea Extract

In the present disclosure a commercial preparation of a Dead Sea extract referred to herein as “Osmoter” or “Osmoter™” or “Mineral Skin Osmoter” was used. The preparations is also known as “Maris Sal” or “Maris Aqua” (Dead Sea Water, DSW) (Source: Geological Survey—Ministry of National Infrastructures, State of Israel, especially for AHAVA-Dead Sea Laboratories CAS # INCI Monograph ID:11089).

The “Osmoter” solution has the following composition:

| Salt normality (N) | | | Salt normality (N) | | |
|---|---|---|---|---|---|
| Na | 0.118 | (2.720 g/l) | Rb | 3.5 × 10⁻⁶ | (<3 × 10⁴ g/l) |
| K | 0.054 | (2.100 g/l) | Sb | <1.6 × 10⁻⁷ | (<2 × 10⁻⁵ g/l) |
| Ca | 0.873 | (35.000 g/l) | Sr | 7.6 × 10⁻³ | (0.670 g/l) |
| Mg | 3.815 | (92.700 g/l) | V | <7.9 × 10⁻⁵ | (<0.004 g/l) |
| Ba | 6.6 × 10⁻⁵ | (0.009 g/l) | Th | <8.6 × 10⁻⁸ | (<2 × 10⁻⁵g/l) |
| Cd | <1.8 × 10⁻⁷ | (<2 × 10⁻⁵ g/l) | U | <8.4 × 10⁻⁸ | (<2 × 10⁻⁵g/l) |
| Co | <3.4 × 10⁻⁵ | (<0.002 g/l) | Zn | <3.06 × 10⁻⁵ | (<0.002 g/l) |
| Cu | <3.15 × 10⁻⁵ | (<0.004 g/l) | | | |
| Cr | <3.85 × 10⁻⁴ | (<0.02 g/l) | Cl | 9.75 | (346 g/l) |
| Fe | <3.58 × 10⁻⁵ | (<0.002 g/l) | Br | 0.175 | (14 g/l) |
| Li | 5.76 × 10⁻³ | (0.040 g/l) | B | 0.011 | (0.120 g/l) |
| Mn | 1.82 × 10⁻⁴ | (0.010 g/l) | As | 2.7 × 10⁻⁵ | (0.002 g/l) |
| Mo | <1.04 × 10⁻⁶ | (<10⁻⁴ g/l) | I | 6.30 × 10⁻⁷ | (8 × 10⁻⁸ g/l) |
| Ni | <3.4 × 10⁻⁵ | (<0.002 g/l) | SiO2 | <3.33 × 10⁻⁴ | (<0.02 g/l) |
| Pb | <9.6 × 10⁻⁸ | (<2 × 10⁻⁵) | SiO4 | <2.2 × 10⁻³ | (<0.2 g/l) |

Solutions comprising Dead Sea Water were prepared by dilutions of the “Osmoter” preparation (See below).

It is noted that the percentages of the Dead Sea extract in the compositions of the present disclosure are provided herein above and below in weight per weight ratio (w/w) i.e., the weight in grams of the Dead Sea extract (e.g., Osmoter) per 100 gram total weight of the composition.

### Example 2: Dunaliella Salina Alga Extract

In the present disclosure a commercial Dunaliella Salina Algae extract PEPHA®-CTIVE was purchased from DSM Nutritional Products Ltd.

PEPHA®-CTIVE is an aqueous extract of the biotechnologically produced microalgae Dunaliella Salina. The extract is rich in amino acids and carbohydrates.

The extract product identification is as follows (Product code: 50 3592 9):

Appearance: clear, yellowish liquid

pH: 4.5-5.5

Relative density d20/20: 1.000-1.050

Residue on drying: >1.5% m/m

Identity by HPTLC: characteristic pattern of ninhydrin positive components; comparable to reference preparation

Total aerobic mesophile plate count: <100 CFU/ml

Specified microorganisms: not detectable in one milliliter

Solubility—PEPHA®-CTIVE is soluble in water.

Composition—as detailed in Table 2 below

TABLE 2
The composition of the Dunaliella Salina Extract product

| INCI name | Content | CAS No. | EINECS/ELINCS*** |
|---|---|---|---|
| Dunaliella Salina | 1-5% | 92128-82-0 | 295-780-4 |
| Extract * | | | |
| Aqua | >50% | 7732-18-5 | 231-791-2 |
| Phenoxyethanol** | 0.1-1% | 122-99-6 | 204-589-7 |
| Sodium | 0.1-1% | 532-32-1 | 208-534-8 |
| Benzoate** | | | |
| Potassium | 0.1-1% | 24634-61-5 | 246-376-1 |
| Sorbate** | | | |

* Previous INCI name: Algae Extract
**preservative
***a standard chemical nomenclature

It is noted that the percentages of the Dunaliella Salina extract in the compositions of the present disclosure are provided herein above and below in weight per weight ratio (w/w) i.e., the weight in grams of the Dunaliella Salina extract per 100 g total weight of the composition.

### Example 3: pRETINOL Complex Preparation

A specific combination of the present application is referred to herein as pRETINOL complex.

The content of the pRETINOL complex (which at times is referred to herein as complex) was as follows:
0.5% Osmoter (OSM)
3.0% Dunaliella Salina ext. (DSE)
0.1% Niacinamide
15.6 ppm β-Carotene
Water (complete to 100%)

The complex was prepared by mixing the various ingredients constituting thereof in water to reach the final concentration as noted. The order of the addition of the ingredients was of no importance.

It is noted that the percentages of the Niacinamide in the compositions of the present disclosure are provided herein above and below in weight per weight ratio (w/w) i.e., the weight in grams of the Niacinamide per 100 gram total weight of the composition.

### Example 4: A Study on Human Skin Organ Culture (HSOC) Demonstrating Safe Retinol Activity of the pRETINOL Complex

As detailed herein below, the study described in the present Example 4 illustrates that pRETINOL complex may contribute to internal skin retinol formation without the risks associated with topically applied Vitamin A1 (also known as retinol) and retinoic acid.

The present Example 4 illustrates that the pRETINOL complex advantageously provides the skin cell with an amount of Beta carotene and Vitamin B3 that assists the cell with producing retinol with safe activity e.g., without the side effects associate with direct application of retinol onto the skin.

Safe retinol activity of the pRETINOL complex of the present invention was demonstrated using ex vivo human skin culture as a representative skin laboratory model.

Cytokines secretion was tested in unstressed skin and following UVB irradiation. The effect of pRETINOL was compared to retinol.

A. Experiment with UVB Stressor

Topical exposure till 48 hours in total (24 hr recovery following topical application+24 hr after exposure to UVB irradiation) with UVB radiation as stressor on human skin model

The following general procedure was used in the study:

Day 1: Topical application of 3 μl of each sample solution on the epidermis of each skin explant. Incubate for 24 hours. SDS 10% was applied only once, and was not farther irradiated with UVB.

Day 2: Skin explants were washed once with PBS and irradiated with UVB lamp at 200 mJ/cm2. After radiation it was incubated for 30 min and then topically apply each treatment (sample solution) again. Control was not irradiated under UVB light. UVB radiated control was without any treatment except radiation.

Day 3: Viability and Caspase3 activity were measured for the epidermis layer. The medium was collected for cytokines test.

Table 3 details the various tested samples:

TABLE 3
tested samples in the experiment conducted with UVB Stressor

| Sample # | Treatment List |
|---|---|
| 1 | No treatment - no UVB |
| 2 | UVB radiation + H₂O |
| 3 | UVB radiation + Complex |
| 4 | UVB radiation + OSM 0.5% |
| 5 | UVB radiation + Dunaliella Salina extract (DSE) 3.0% |
| 6 | UVB radiation + Niacinamide 0.1% |
| 7 | UVB radiation + β-Caroten 15.6 ppm |
| 8 | UVB radiation + Retinol 0.05% (w/w) in EtOH |
| 9 | UVB radiation + Retinol 0.5% (w/w) in EtOH |
| 10 | UVB radiation + Retinol 1.0% (w/w) in EtOH |
| 11 | UVB radiation + EtOH |
| 12 | SDS 10% (applied only once) |

For all of the assays in this Example 4 (*) denotes values that are significantly different from the Untreated tissues p<0.05; (**) denotes values that are significantly different from the Untreated tissues p<0.01; and (*) denotes values that are significantly different from the Untreated tissues p<0.001.

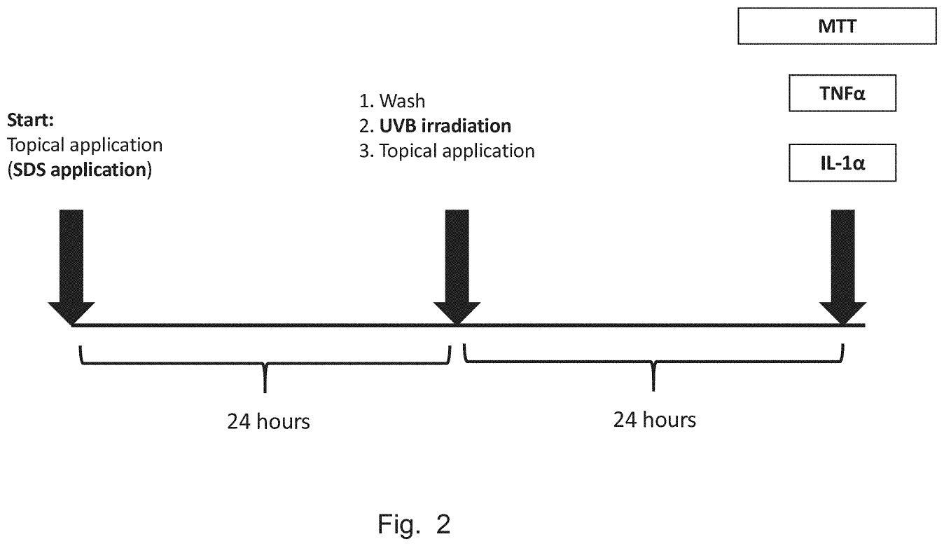

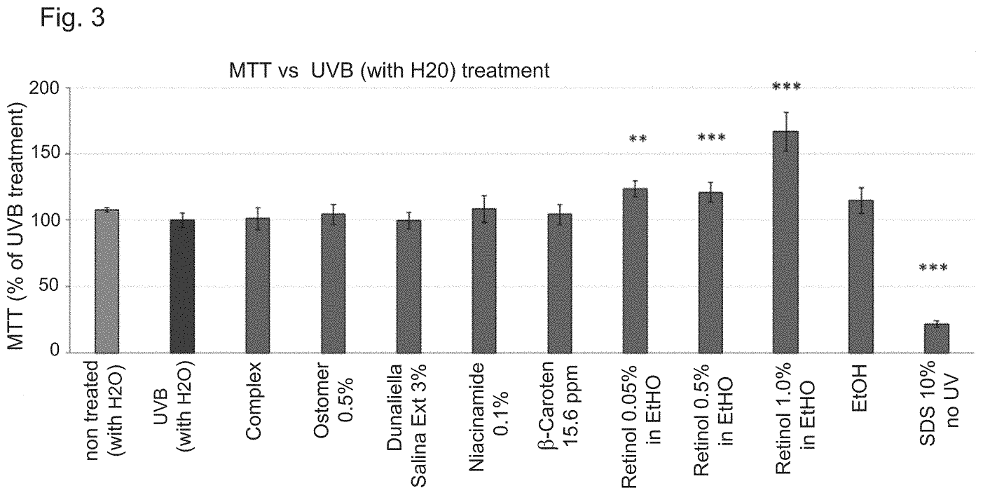

FIG. 2 illustrates a flow diagram of topical exposure/treatment till 48 hours, 24 hr recovery following topical application +24 hr after exposure to UVB irradiation) with UVB radiation as stressor on human skin model experiment. In the Experiment the SDS irritation control sample was a skin explant that was topical applied with 3 μl SDS 10% solution at day 1 only.

The viability of the various skin explants was measured by the 5-dimethylthiazol-2-yl]-2,5-diphenyl tetrazolium bromide (MTT) assay.

FIG. 3 illustrates the viability results as observed with 3 to 6 repetition of the tests. The results in FIG. 3 illustrate the following:
Topical treatment with the 10% SDS sample only once caused epidermis death after 24 hours of incubation.
Topical treatment with Retinol resulted with increased mitochondrial activity (dehydrogenases) in the epidermis.
The rest of the treatment samples, including the pRETINOL complex did not show changes in the viability parameters compared to the irradiated control sample.

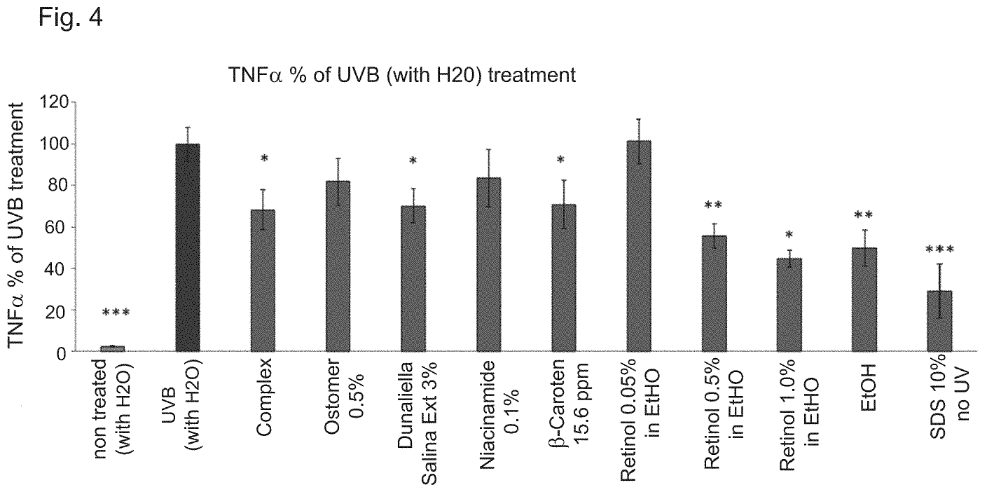

FIG. 4 illustrates the TNFα secretion results in the medium as observed with 2 to 4 repetition of the tests. In FIG. 4 the TNFα secretion of the irradiated sample “UVB” was selected as 100% and it equals 53±30 pg/ml.

The results in FIG. 4 illustrate the following:
The negative control (without radiation, the “non-treated” sample) illustrated a concentration of the TNFα which is 43 times lower than that of an irradiated sample (“UVB” sample).
Topical treatment with the pRETINOL complex, the Dunaliella extract and the β-Caroten sample illustrated significant reduction in the TNFα secretion compared to the irradiated sample. After treatment the TNFα concentration was 70% of the irradiated skin explant.
Topical treatment with Retinol only at concentrations of 0.5% and 1.0% significantly reduced the TNFα secretion compared to the irradiated sample by 56% and 45%, respectively.
Topical one treatment with 10% SDS caused a significant reduction in the TNFα secretion that resulted from the drastic reductions of viability.

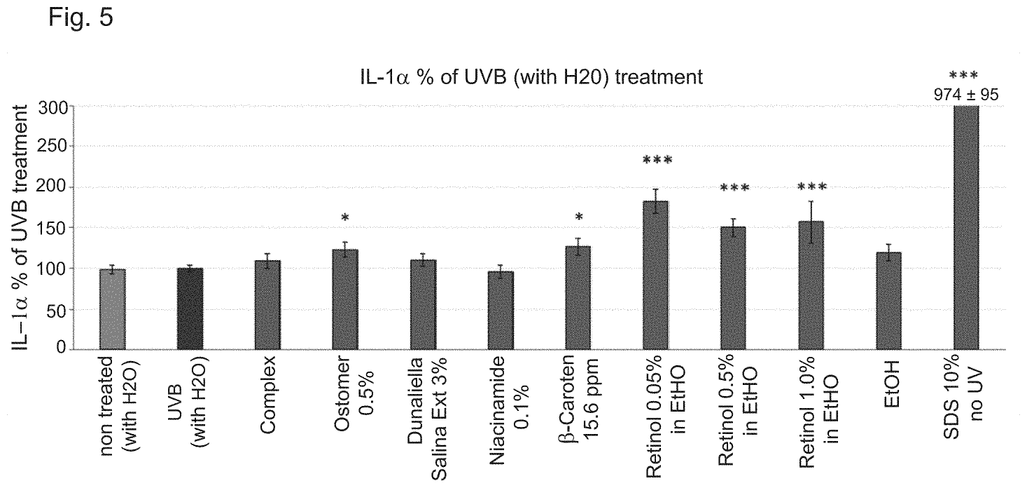

FIG. 5 illustrates the IL-1α secretion results in the medium as observed with 3 to 4 repetition of the tests. In FIG. 5 the IL-1α secretion of the irradiated sample “UVB” was selected as 100% and it equals 8.1±1.0 pg/ml.

The results in FIG. 5 illustrate the following:
UVB radiation did not affect the IL-1α cytokine in the skin model.
Topical treatment with Retinol only at concentrations of 0.05%, 0.5% and 1.0% significantly increased the IL-1α secretion (83%, 50% and 53%, respectively).
Topical treatment with 10% SDS dramatically increased the IL-1α secretion after incubation of 24 hours.
No difference in the IL-1α secretion was observed after topical treatment with the pRETINOL complex.
Topical treatment with the Osmoter and the β-Caroten samples illustrated an increase of 20% in the IL-1a secretion compared to the control.

Conclusions:

The results of topical exposure till 24 hr following UVB exposure as stressor on human skin model illustrate the following:
Topical application of the pRETINOL complex and 0.5% and 1.0% Retinol samples illustrated a reduction in the TNFα secretion.
Topical application of the pRETINOL complex did not cause an increase in the IL-1α cytokine secretion.
Topical application of Retinol samples at all studied concentrations caused an increase in the IL-1α cytokine secretion. Without wishing to be bound by theory, the inventors believe that this observed increased secretion of IL-1α may be due to irritation effect of the Retinol samples on the skin explants.

In can be seen from the skin model experiment performed with UVB stressor that while the control samples of Retinol (particularly the 1.0% sample) clearly have biological effects, the pRETINOL complex illustrated a clear advantage due to lack of skin irritation.

It is noted that retinol (0.5% and 1%) and pRETINOL complex show protection from UVB inflammation by attenuation of the inflammatory cytokine TNFα-induced by UVB. However, when testing IL-1—a cytokine-induced by UVB and known also as irritation marker, it can be seen that only pRETINOL complex significantly attenuated IL-1α, while Retinol (0.05%, 0.5%, 1%) not only did not attenuated IL-1α, but even enhanced its secretion following irradiation.

B. Experiment without External UVB Stressor

Topical Exposure Till 96 Hours without External UVB Radiation as Stressor on Human Skin Model

The following general procedure was used in the study:

Day 1: Topical application of 3 μl of each solution on the epidermis of each skin explant. Incubate for 24 hours. (SDS Irritation control: topical application of 3 ul SDS 10% was applied on the skin model at day 1 only. The rest of the days samples were untouched).

Day 2: Topical application of 3 μl of each solution on the epidermis of each skin explant. Incubate for 24 hours in fresh medium (medium was not collected). (UVB control: after 24 hours incubation with 3 μl H₂O skin was irradiated with 200 mJ/cm2 UVB. The rest of the experiment days sample were topically applied with H₂O, as control).

Day 3: Topical application of 3 μl of each solution on the epidermis of each skin explant. Incubate for 24 hours in fresh medium (medium was collected)—48 hours of total topical incubation.

Day 4: Topical application of 3 μl of each solution on the epidermis of each skin explant. Incubate for 24 hours in fresh medium (medium was collected)—72 hours of total topical incubation.

Day 5: Topical application of 3 μl of each solution on the epidermis of each skin explant. Incubate for 24 hours in fresh medium (medium was collected)—96 hours of total topical incubation. MTT assay was performed.

Table 4 details the various tested samples:

TABLE 4
tested samples in the experiment conducted
without external UVB Stressor

| Sample # | Treatment List |
|---|---|
| 1 | H₂O |
| 2 | Complex |
| 3 | OSM 0.5% |
| 4 | Dunaliella Salina extract (DSE) 3.0% |
| 5 | Niacinamide 0.1% |
| 6 | β-Caroten 15.6 ppm |
| 7 | Retinol 0.05% (w/w) in EtOH |
| 8 | Retinol 0.5% (w/w) in EtOH |
| 9 | Retinol 1.0% (w/w) in EtOH |
| 10 | EtOH |
| 11 | SDS 10% (applied only once) |
| 12 | UVB radiation + H₂O |

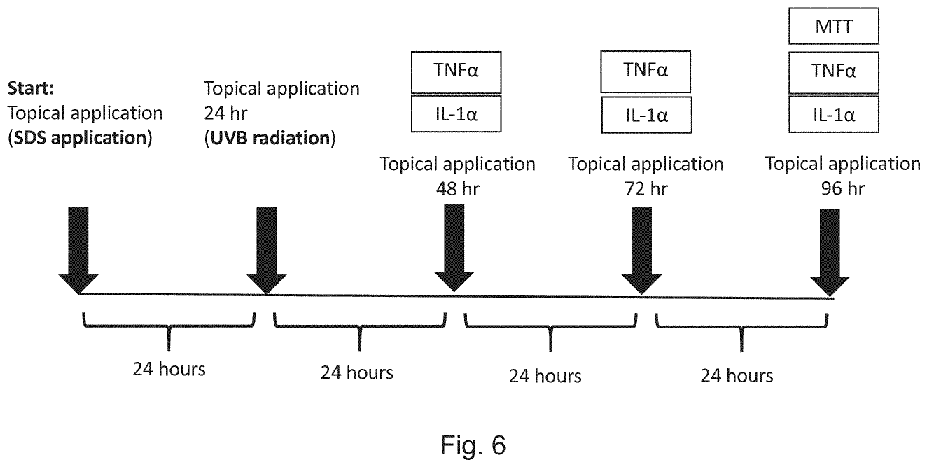

FIG. 6 illustrates a flow diagram of topical exposure/treatment till 96 hours without external UVB radiation as stressor on human skin model experiment. In the Experiment the SDS irritation control sample was a skin explant that was topical applied with 3 μl SDS 10% solution at day 1 only (the rest of the days the SDS sample was untouched).

The viability of the various skin explants was measured by the MTT assay.

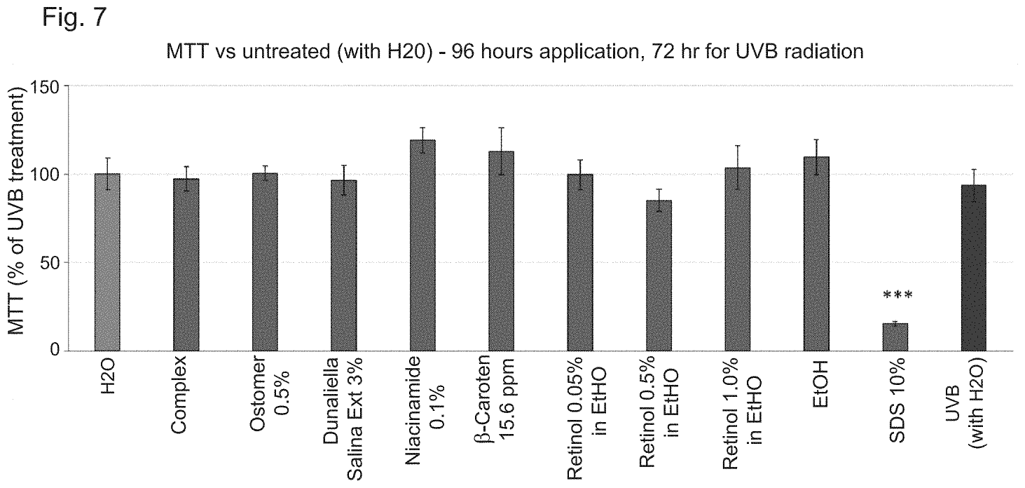

FIG. 7 illustrates the viability results as follows:
Topical treatment with the 10% SDS sample only once caused a significant decrease in the epidermis viability after 96 hours to only 15% of the control value
The rest of the treatment samples illustrated mitochondrial activity identical to the of the control.

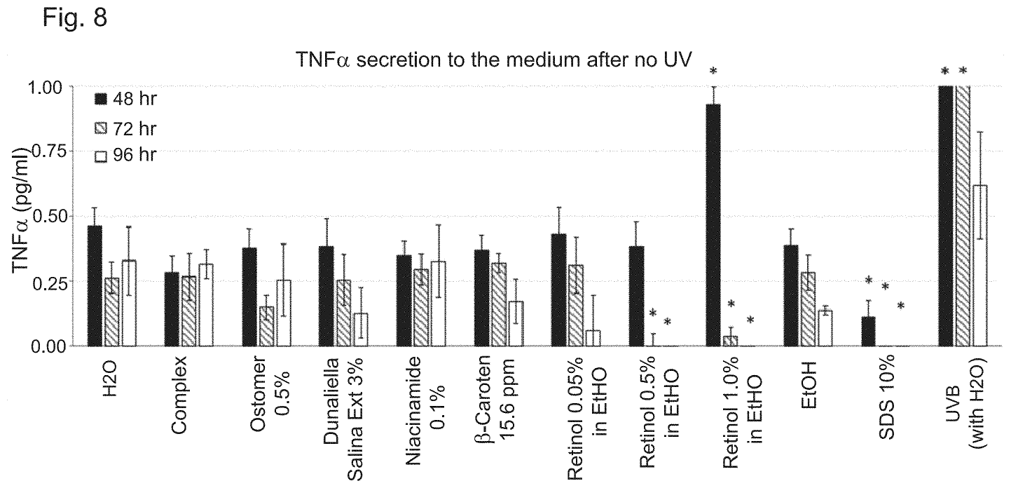

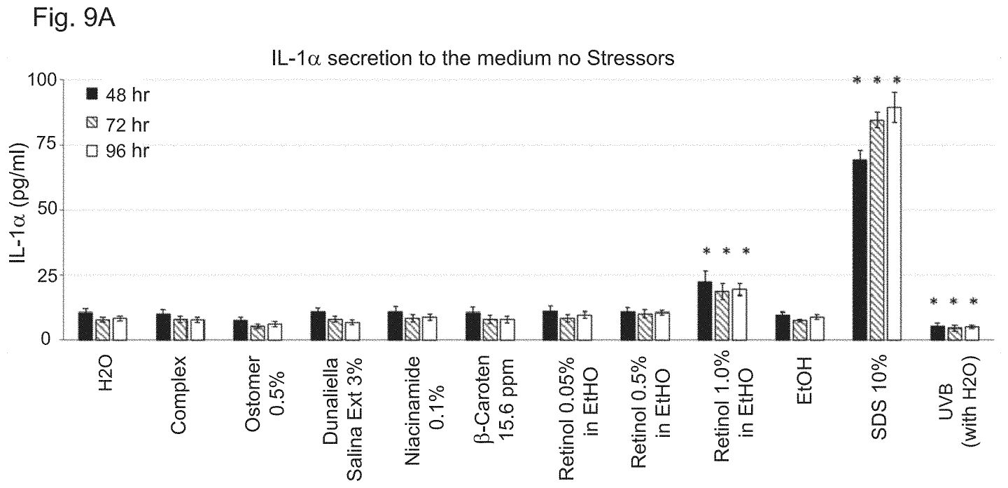

FIG. 8 illustrates the TNFα secretion tested results. In FIG. 8 the TNFα secretion of the H₂O sample was used as a control (0.35±0.05 pg/ml) for each time point.

The results in FIG. 8 illustrate the following:
Topical treatment with Retinol only at concentrations of 0.5% and 1.0% reduced the TNFα secretion below the detection level (which was very sensitive) after incubation of 72 hours.
Topical treatment with 10% SDS caused a significant reduction in the TNFα secretion below the detection lever already after incubation of 48 hours.
Treatment with UVB radiation caused a significant increase in the TNFα secretion 24 hours after irradiation, wherein a reduction from the increased level to a value similar to that of the control value after 96 hours was observed.

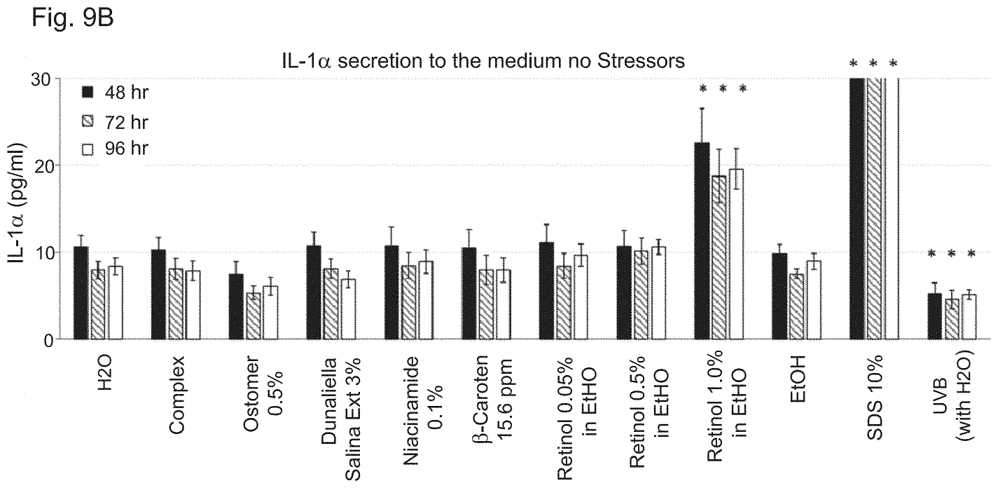

FIG. 9A illustrates the IL-1α secretion results in the medium. In FIG. 9A the IL-1α secretion of the H₂O sample was used as a control (9.0±0.7 pg/ml 0.35±) for each time point. FIG. 9B is a magnification of FIG. 9A.

The results in FIGS. 9A and 9B illustrate the following:
Topical treatment with Retinol only at concentrations of 1.0% increased the IL-1α secretion after 48 hours of incubation, then after the IL-1α level remind the same until 96 hours.
Surprisingly, topical treatment with 10% SDS increased the IL-1α secretion level to a value which is 6.5 times of the control value after 48 hours, with continued increase to 10.6 times of the control value after 72 hours and 96 hours (it is noted that visual inspection of the epidermis after 24 hours indicated no viability thereof).
UVB radiation caused a reduction in the IL-1α cytokine secretion.

Conclusions:

The results of topical exposure till 96 hours of human skin model, without UVB radiation, illustrate the following:
After 96 hours of topical application, in terms of safety, both Retinol and the pRETINOL complex did not cause increase or reduction of viability of the skin explants.
Topical exposure to Retinol for 48, 72 and 96 hours caused a reduction in the TNFα secretion. The reduction was also observed with topical treatment with alcohol (ethanol) only.
Topical exposure (exposure of 48, 72 and 96 hours) to 1.0% Retinol caused an increase in the IL-1α cytokine secretion. Without wishing to be bound by theory, the inventors believe that the increase of IL-1α secretion at the high concentration of Retinol (i.e., 1.0%) may be due to the irritation induction known tendency of Retinol.
Topical application of the pRETINOL complex did not affect the level of the IL-1α and TNFα cytokine compared to the control.

### Example 5: Gene Expression for Relevant Biological Pathway—Microarray Results in Skin Equivalents—A Study Demonstrating Effective Biological Activity of pRETINOL Complex

As detailed herein below, the study described in the present Example 5 illustrates that pRETINOL complex has effective biological activity.

Biological activity on gene expression was performed using DNA microarray. The pRETINOL complex was tested and compared to each one of the individual ingredients comprised therein.

### A. DNA Microarray Procedure

### Purpose

DNA microarrays can be used to screen for changes in the expression of hundreds to thousands of different genes, depending upon the gene chip set selected for the study.

### Summary of Test Method

DNA microarrays are extremely powerful tools that allow users to analyze changes in gene expression by monitoring the RNA products of thousands of genes in a single experiment. Microarrays come in many forms, however the most popular form are comprised of glass microscope slides which have been studded with a large number of DNA fragments, also called features, with each feature containing a nucleotide sequence that corresponds to a single specific gene. DNA microarrays are commonly used to compare treated and untreated cells/tissue to determine what changes in gene expression occur with the treatment.

### Methods

Treatment

MatTek EFT-400 full thickness tissues were used as the model for this study. Upon arrival, the full thickness tissues were placed into 6 wells plates with 2 ml of culture media and incubated overnight at 37±2° C. and 5±1% CO₂. After this overnight incubation the culture media was replaced with 5 ml of fresh media and the tissues were treated topically with the test materials prepared in distilled water as follows:

Table 5 details the various tested samples:

TABLE 5
tested samples in the microarray Procedure

| Sample # | Treatment List |
|---|---|
| 1 | Untreated control |
| 2 | 15.6 ppm β-Caroten |
| 3 | 3.0% Dunaliella Salina extract |
| 4 | 0.1% Niacinamide |
| 5 | 0.5% Osmoter |
| 6 | Complex (0.5% Osmoter; 3% Dunaliella Salina |
| | extract; 15.6 ppm β-Caroten; 0.1% Niacinamide) |
| 7 | Distilled Water |

The following Test Materials were used in preparing the aforementioned tested samples:

Test Material Identification: Beta Carotene (10%)

Physical Description: Red/Orange Powder

Dilutions Tested: 15.6 ppm

Test Material Identification: Dunaliella Salina Extract

Physical Description: Faint Tan, Clear Liquid

Dilutions Tested: 3%

Test Material Identification: Niacinamide

Physical Description: White Powder

Dilutions Tested: 0.10%

Test Material Identification: Osmoter

Physical Description: Colorless, Clear Liquid

Dilutions Tested: 0.5%

After the application of the test materials the tissues were incubated at 37±2° C. and 5±10% CO₂ for 48 hours.

Total RNA Isolation (Ambion RNAqueous Kit)

At the end of the treatment period, the tissues were rinsed and transferred to a 2 ml centrifuge tube containing 700 μl of lysis buffer and homogenized. After centrifuging at 14,000×g for 10 minutes at 4° C. the supernatant from each tube was transferred to a new 1.5 ml tube and mixed with and equal volume of 64% ethanol. After mixing the solutions was transferred to glass fiber filter cartridges and the cartridges were loaded into a 1.5 ml collection tube centrifuged for 1 minute at 14,000 RPM in a Napco 2002 Microcentrifuge with a DA-6T fixed angle rotor. The flow through was discarded and any remaining mixture was loaded into the filter cartridge and the centrifugation process was repeated until all of the mixture had been processed. The filter was then washed to remove any residual cellular debris from the RNA bound to the glass fibers by subsequently applying 700 μl of wash solution 1 (1 time) and 500 μl of wash solution 2 (2 times) to the filter cartridge and centrifuging at 14,000 RPM for 1 minute to pass each wash through the cartridge. After each wash, the flow through was discarded. After the final wash one final spin was performed without wash solution to remove any residual wash solution in the filter cartridge. The RNA bound to the glass fibers within the cartridge was then eluted by applying 40 μl of TE buffer (10 mM Tris-HCl, 1 mM EDTA, preheated to 70-80° C.) to the cartridge and centrifuging the cartridge in a new collection tube at 14,000 RPM for one minute. The elution process was then repeated a second time using 20 μl of TE buffer.

mRNA Amplification (Ambion MessageAmp aRNA kit)

First Strand cDNA Synthesis: To start the first strand synthesis, 10 μl of total RNA for each sample was added to a 600 μl PCR tube and the total volume of liquid in the tube will be adjusted to 11 μl with DEPC H₂O. Next, 1 μl of T7 Oligo(dT) primer was added and the tube was incubated in a water bath at 70±2° C. for 10 minutes to denature the RNA and then placed on ice to allow the primers to anneal to the poly A ends of the mRNA. After cooling 2 μl of 10× first strand buffer, 1 μl of RNAse inhibitor and 4 μl of dNTP mix were added to each tube, and the tube was incubated at 42° C. in a hybridization oven (Labnet Problot). As soon as the tube was heated, 1 μl of Reverse Transcriptase was added and the tubes were returned to 42±2° C. for 2 hours. At the end of the two hours the tubes were briefly centrifuged to collect all of the fluid at the bottom of the tube and then placed on ice.

Second Strand Synthesis and cDNA Purification: For the synthesis of the second strand of cDNA the following items were added to the tubes above (in this order): 63 μl DEPC H₂O, 10 μl 10× second strand buffer, 4 μl dNTP mix, 2 μl DNA Polymerase and 1 μl of RNAse H. The tubes were mixed and then incubated at 16±2° C. for 2 hours in a refrigerated centrifuge chamber (Precision Durafuge 300R with the rotor removed). Towards the end of the 2 hour incubation a sufficient quantity of DEPC H₂O was warmed to 50±2° C. in a water bath and a cDNA purification filter cartridge was equilibrated with 50 μl of cDNA binding buffer (one cartridge per sample) for at least 5 minutes. After the samples finished incubating 250 μl of cDNA binding buffer was added to each tube and thoroughly mixed. The contents of the PCR tube were then transferred to the cDNA purification filter cartridge. The cartridge was then placed in a collection tube and centrifuged at 10,000 RPM for 1 minute. The flow-through was discarded and 650 μl of cDNA wash solution was added to the cartridge. The cartridge was centrifuged again and the flow-through was discarded, and then centrifuged one last time to ensure that the wash buffer had been completely emptied from the filter. The cDNA was eluted by applying 10 μl of preheated DEPC H₂O to the filter and centrifuging the filter in a new collection tube at 10,000 RPM for one minute. This elution was performed one additional time to give a total volume of 16-18 μl of recovered cDNA solution.

In Vitro Transcription to Synthesize aRNA and aRNA Purification: The in vitro transcription began by adding the following to the cDNA solution prepared above: 4 μl each of T7 ATP solution, T7 CTP solution, T7 GTP solution, T7 UTP solution, 4 μl of 10×Reaction buffer, and 4 μl of T7 enzyme mix. The tube was mixed and then incubated at 37±2° C. for 6-14 hours in a hybridization oven. Towards the end of the incubation a sufficient volume of Elution Solution was warmed to 50-60° C. and an aRNA filter cartridge was equilibrated with 100 μl of aRNA binding buffer for at least 5 minutes. At the end of the incubation period, 350 μl of aRNA binding buffer was added to the sample tubes and thoroughly mixed. An additional 250 μl of absolute ethanol was also added to each tube. The mixture was then transferred to an aRNA filter cartridge; the cartridge was inserted into a collection tube and centrifuged at 10,000 RPM for 1 minute. The flow-through was discarded and 650 μl of aRNA wash buffer was added to the cartridge followed by centrifuging at 10,000 RPM for one minute. After discarding the flow through the cartridge was spun one final time to remove all traces of the wash buffer. The cartridge was then transferred to a new collection tube and 40 μl of pre-warmed Elution Solution was added to the cartridge. The cartridge was incubated for 2 minutes at room temperature and then the aRNA was eluted by centrifuging for 1 minute at 10,000 RPM. This elution was performed one additional time to give a total volume of 80 μl of aRNA solution. The final concentration of the aRNA was determined by the Ribogreen assay described herein. In addition, the quality of the aRNA was checked via gel electrophoresis as described below.

RNA Concentration Assay (Molecular Probes Ribogreen Assay)

The Ribogreen reagent is provided as a stock solution in DMSO. Prior to use the reagent was diluted 2000 fold in TE buffer. The RNA assay requires 200 μl of diluted Ribogreen reagent per sample to be tested and 1 ml of the reagent for the standards. A series of RNA standards was prepared by diluting purified ribosomal RNA derived from E. coli to the following concentrations: 1 μg/ml, 0.5 μg/ml, 0.1 μg/ml, 20 ng/ml and 0 ng/ml (blank). Prior to assaying, one microliter of the RNA samples prepared above was diluted 1000 fold in TE buffer. For the RNA assay, 100 μl of the diluted samples or standards was transferred to the wells of a black 96-well plate. The samples and standards were assayed in duplicate. After the samples/standards were added to the plate 100 μl of the diluted Ribogreen assay reagent was added and the plate was gently mixed and allowed to incubate for 5-10 minutes protected from the light. After this incubation the plate was read with a Thermo Labsystems Fluorskan Ascent FL fluorometer using an excitation wavelength of 500 nm and an emission wavelength of 525 nm.

Labeling of aRNA with Fluorescent Dyes (PerkinElmer ASAP RNA Labeling Kit)

Two tubes were prepared for the labeling process, one for Cy3 labeling (green) and one for Cy5 labeling (red). To the Cy3 tube 2 μg of aRNA prepared from the untreated/control sample (the actual color assignment for each sample is not important, but for consistency Cy3 is normally used for the untreated sample) and enough DEPC H₂O was added to bring the total volume up to 4 μl. To the Cy5 tube 2 μg of aRNA prepared from the sample treated with the test material and enough DEPC H₂O was added to bring the total volume up to 4 μl. To both tubes 5 μl of ASAP labeling buffer was added along with 1 μl of the specific dye for the tube (Cy3 or Cy5). The tubes were incubated for 15 minutes at 85±2° C. At the end of the 15 minutes the tubes were placed on ice to cool them and then add 2.5 μl of ASAP stop solution was added to each tube.

Purification of Labeled aRNA

To purify the labeled aRNA, a Millipore Microcon YM-30 filter column was inserted into a collection tube and filled with 400 μl of TE buffer. The Cy3 and Cy5 probes were combined (6 μl of each or approximately 1 μg of each labeled aRNA) and then added to the microcon filter and thoroughly mixed with the TE buffer. The filter was centrifuged at 12,000 RPM for 8 minutes and the flow through was discarded. The column was then washed twice with 400 μl of TE buffer, discarding the flow though after each centrifugation (12,000 RPM for 8 minutes). After the final wash the filter column was inverted, placed into anew collection tube and centrifuged at 12,000 RPM for 2 minutes to collect the probe (the probe was concentrated in a volume of 2-30 μl of residual TE buffer).

Microarray Hybridization and W Ashing (Agilent Technologies Microarrays)

For hybridization, 11 μl of 10× control target RNA (supplied with Agilent Technologies In Situ Hybridization Kit) was mixed with 30 μl of DEPC water and 2.5 μl of 25× Agilent Fragmentation Buffer. This mixture was allowed to incubate at 65° C. for approximately 30 minutes in a hybridization oven. At the end of the incubation 55 μl of Agilent Hybridization Buffer was added along with the fluorescent aRNA probes prepared above. An Agilent SUREHYB hybridization chamber was prepared by inserting a glass gasket slide into the bottom half of the chamber and the hybridization mixture (approximately 110 μl) was applied to the glass gasket slide and a custom Agilent DNA Microarray Chip was placed face down on top of this gasket such that the hybridization solution was sandwiched between the glass gasket slide and the microarray face of the chip. The top half of the chamber was then attached and the connecting thumbscrew was tighten. After verifying that there was good bubble formation in the chamber, it was placed into the hybridization oven for approximately 17 hours (65° C. and rotating at 4 RPM). At the end of the hybridization period the microarrays/glass gasket were removed from the SUREHYB chamber and placed in 50 ml of wash solution 1 (room temperature, 6×SSC, 0.005% Triton X-102). After the gasket had fallen away from the microarray, the array was placed in 300 ml of fresh wash solution 1 on a magnetic stir plate. The array was washed while the solution was mixed at medium speed for 10 minutes and then transferred to 300 ml of wash solution 2 (0.1×SSX, 0.005% Triton X-102, 4° C.) for 5 minutes. After the final wash the array was dried by centrifuging at 500 RPM for 5 minutes.

Microarray Scanning and Analysis

The microarrays were scanned with an Axon GenePix 4100A Scanner with the scanning resolution set to 5 μm and analyzed with GenePix Pro software. During the initial scan the PMT gains for the scanner were adjusted such that the cy5/cy3 image count ratios were between 0.95 and 1.05.

Calculations

Microarray Calculations

Fluorescence intensities for the microarrays were subjected to global normalization. The total fluorescent signal for both dyes were normalized to one to established a correction factor that would make the total intensities for both dyes equal. Criteria for evaluating changes in gene expression will vary from study to study however typically three criteria are required:

1. The ratio of Cy3/Cy5 (treated/untreated) fluorescence intensity is greater than 1.3 or less than 0.66. This relates to a change in gene expression of at least +/−30%.

2. The fluorescence intensity of the gene marker is greater than the background intensity.

Results

After scanning the arrays, the results were exported to an Excel spreadsheet. On each array, treated vs. control were compared. Each array was processed and the data was normalized from all arrays to get the average expression and log fold change (log FC—a known bioinformatics calculation method) for each gene on each array.

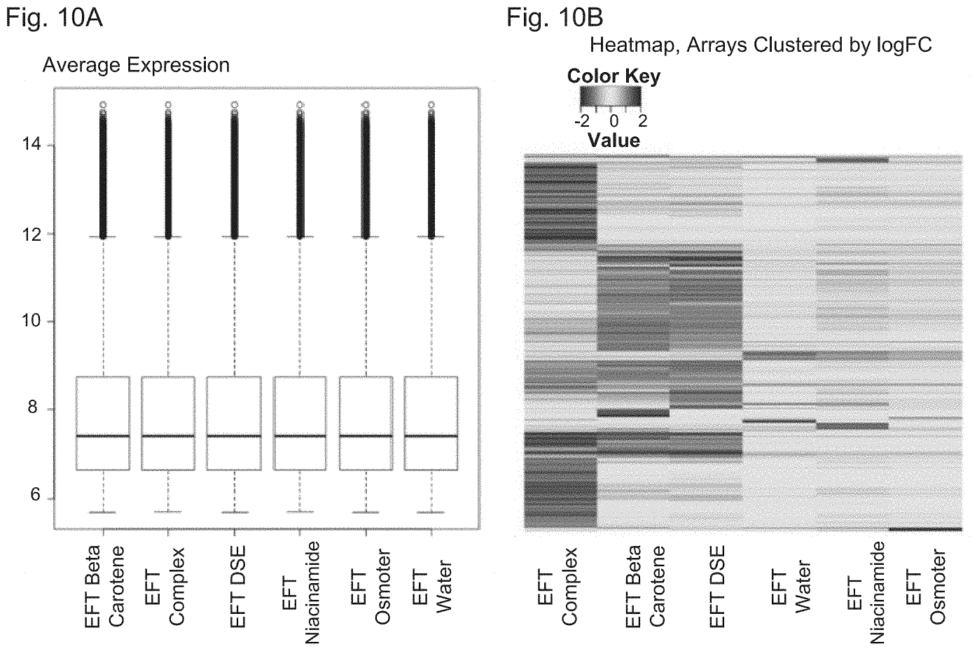

FIG. 10A illustrates the averaged expression. FIG. 10B illustrates the Heatmap, Arrays Clustered by log FC. From the heatmap, it can be seen that EFT Beta Carotene and EFT DSE are very similar with several increased (red) or decreased (blue) genes (colors not shown in grayscale figure). EFT Niacinamide and EFT Osmoter both have few changed genes, and they are similar to EFT water. EFT Complex has a different pattern, it shares some of the changed genes with EFT Beta Carotene and EFT DSE, but has its own group of increased and decreased genes.

The number of up- or down-regulated genes for each array is listed in Table 6 below.

TABLE 6
Down and Up regulated genes

| Experiment | Up-Regulated Genes | Up-Regulated Genes |
|---|---|---|
| EFT Beta_Carotene | 196 | 503 |
| EFT Complex | 443 | 407 |
| EFT DSE | 229 | 534 |
| EFT Niacinamide | 133 | 70 |
| EFT Osmoter | 90 | 22 |
| EFT Water | 103 | 9 |

Summary of Differentially Expression Genes (DEGs)

The following is a summary of differentially expression genes (DEGs) for the Beta Carotene sample (the data for the other tested samples is not shown): A total of 19254 genes were evaluated on the array for experiment EFT Beta Carotene. For a single array data the fold change can be obtained and the expression level, but not statistical significance. Since genes with high expression levels tend to be more reliably detected, and genes with low expression tend to give less reliable results from array, the log FC was adjusted by a factor using equation Expression/Median (range from 0.5 to 2). The adjusted Fold change was used to rank genes for Gene set enrichment analysis (GSEA) (Subramanian et al., PNAS 2005) [17]. From those genes, 196 are considered as up-regulated and 503 are considered as down-regulated using a cut off of two fold change from the adjusted Fold Change values.

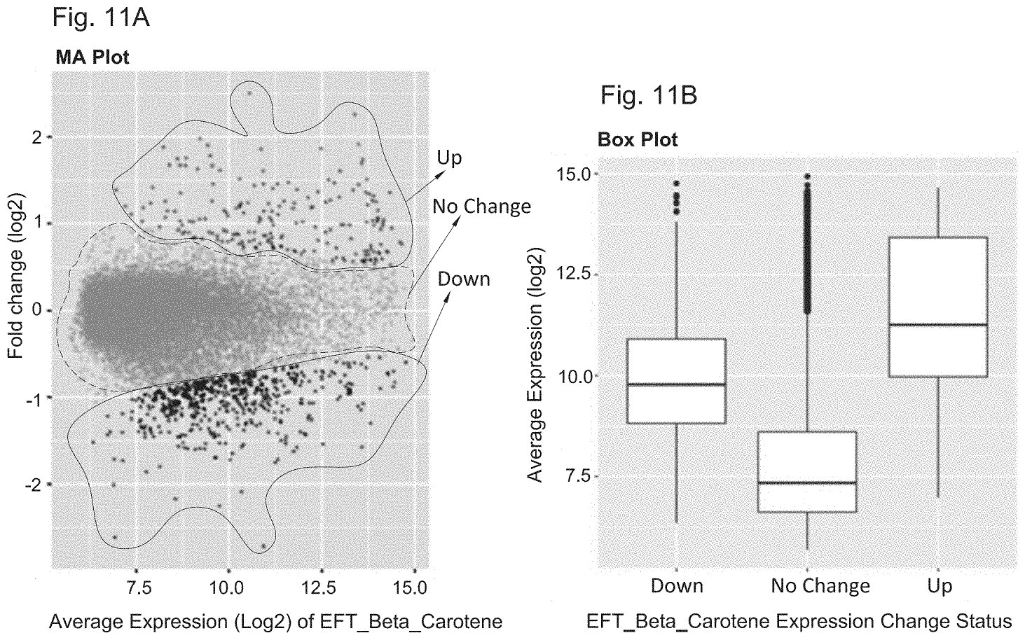

FIG. 11A illustrates an MA plot received for the Beta Carotene tested sample. The MA plot shows a fold change versus average expression on the y- and x-axes, respectively. From the plot the genes that display large-magnitude change and their expression levels are visualized. The up and down regulated genes are labeled in the figure as well as those with no change.

FIG. 11B illustrates the obtained Box Plot received for the Beta Carotene tested sample. The Box Plot shows the distribution of expression levels for genes that are up, down or unchanged. Often genes with significant changes also tend to have higher expression levels.

To shed light on what biological pathways might be affected in the experiment, two methods were used to perform functional enrichment analysis. First, GSEA and all the genes ranked by adjusted log FC as input to GSEA. Second, a focus on the changes genes (up-regulated and down-regulated) was made and functional categories were looked for that show up more often in changed genes versus the whole genome (i.e., enriched).

A group of 32 pathways manually selected from KEGG and wiki-pathways were analyzed in detail. Bioconductor piano package [18] was used to run PAGE enrichment of selected pathways using the log FC data from the arrays. From the PAGE analysis, each pathway is assigned a Z-sore and associated p-value and FDR values. A positive Z-score means that the pathway tends to be up-regulated, a negative Z-score means that the pathway tends to be down-regulated. Larger absolute value of Z-score, as well as small p-values mean more significant regulation.

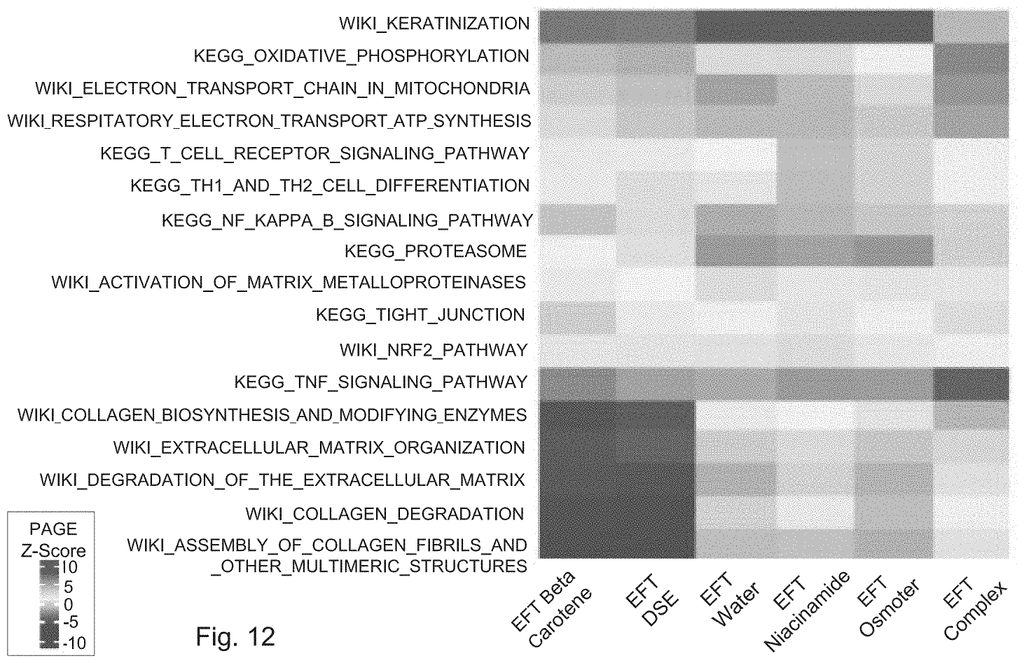

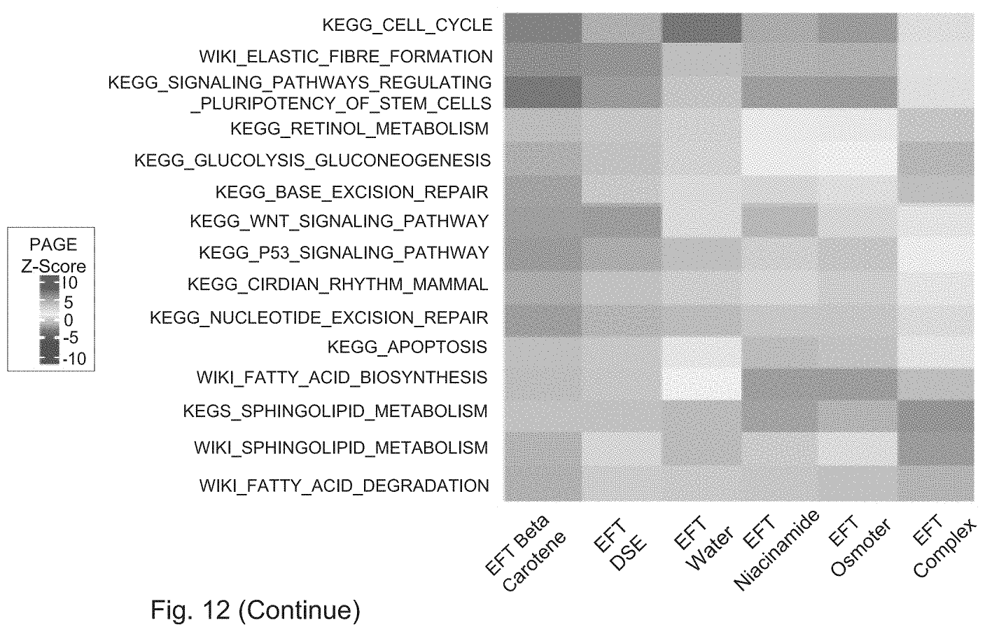

FIG. 12 illustrates a Heatmap showing all z-scores for 32 pathways selected from KEGG and wiki-pathways. From the Z-Score heatmap it can be seen that several pathways are very much down regulated [dark blue, see left down corner of the Heatmap (colors not shown in grayscale figure)] in EFT Beta Carotene and EFT DSE. These includes collagen synthesis and extracellular matrix:
WIKI_COLLAGEN_BIOSYNTHESIS_AND_MODIFYING_ENZYMES
WIKI_EXTRACELLULAR_MATRIX_ORGANIZATION
WIKI_DEGRADATION_OF_THE_EXTRACELLULAR_MATRIX
WIKI_COLLAGEN_DEGRADATION
WIKI_ASSEMBLY_OF_COLLAGEN_FIBRILS_AND_OTHER_MULTIMERIC_STRUCTURES

In contrary, WIKI_KERATINIZATION pathway that is up-regulated after treatment with EFT_Niacinamide, EFT_Osmoter and EFT_Water, remains unchanged after treatment with EFT complex.

Log FC data for genes in individual pathways was created (data not shown) and the log FC values were used to generate heatmaps.

Table 7 represents a numeric data of the z-scores presented in FIG. 12. The bold marked pathways are those that illustrated different expression direction between the complex and its components.

TABLE 7
a numeric data of the z-scores presented in FIG. 12.

| | Beta- | | |
|---|---|---|---|
| Biological pathway | Carotene | Complex | DSE |
| KEGG_SPHINGOLIPID_METABOLISM | −0.47731 | −1.8515 | −0.47158 |
| KEGG_CELL_CYCLE | −2.4656 | 0.41174 | −1.117 |
| KEGG_NF_KAPPA_B_SIGNALING_PATHWAY | 3.1522 | 2.5956 | 2.1651 |
| KEGG_TNF_SIGNALING_PATHWAY | 5.6735 | 7.0414 | 4.6776 |
| KEGG_T_CELL_RECEPTOR_SIGNALING_PATHWAY | 1.5111 | 0.95338 | 1.4808 |
| KEGG_SIGNALING_PATHWAYS_REGULATING_PLURIPOTENCY_OF_STEM_CELLS | −2.818 | 0.48116 | −1.701 |
| KEGG_CIRCADIAN_RHYTHM_MAMMAL | −1.209 | 0.63974 | −0.45414 |
| KEGG_TIGHT_JUNCTION | −0.073579 | 0.40481 | 0.79209 |
| KEGG_GLYCOLYSIS_GLUCONEOGENESIS | −1.0754 | −0.8838 | −0.43146 |
| KEGG_FATTY_ACID_BIOSYNTHESIS | −0.61851 | −0.58772 | −0.30542 |
| KEGG_FATTY_ACID_DEGRADATION | −1.0292 | −0.94996 | −0.10005 |
| KEGG_TH1_AND_TH2_CELL_DIFFERENTIATION | 1.3936 | 1.2331 | 0.34762 |
| KEGG_APOPTOSIS | −0.56651 | 0.60171 | −0.29779 |
| KEGG_OXIDATIVE_PHOSPHORYLATION | 3.5721 | −2.2601 | 4.0391 |
| KEGG_NUCLEOTIDE_EXCISION_REPAIR | −1.5901 | 0.38332 | −0.91134 |
| KEGG_BASE_EXCISION_REPAIR | −1.4296 | −0.55741 | −0.20394 |
| KEGG_P53_SIGNALING_PATHWAY | −1.6942 | 1.3252 | −1.2123 |
| KEGG_PROTEASOME | 1.1865 | −0.019661 | 1.9931 |
| KEGG_RETINOL_METABOLISM | −0.63771 | −0.35064 | −0.31956 |
| KEGG_WNT_SIGNALING_PATHWAY | −1.5459 | 0.61254 | −1.6175 |
| WIKI_COLLAGEN_BIOSYNTHESIS_AND_MODIFYING_ENZYMES | 4.7943 | −0.85388 | −4.4194 |
| WIKI_ASSEMBLY_OF_COLLAGEN_FIBRILS_AND_OTHER_MULTIMERIC_STRUCTURES | −5.9295 | 1.9216 | −5.2921 |
| WIKI_COLLAGEN_DEGRADATION | −5.0219 | 1.2313 | −5.12 |
| WIKI_ACTIVATION_OF_MATRIX_METALLOPROTEINASES | 0.82439 | 1.6771 | 1.116 |
| WIKI_DEGRADATION_OF_THE_EXTRACELLULAR_MATRIX | 4.6457 | 1.9978 | −4.7939 |
| WIKI_EXTRACELLULAR_MATRIX_ORGANIZATION | 4.5653 | 2.4171 | −3.8458 |
| WIKI_ELASTIC_FIBRE_FORMATION | −2.0745 | 1.9169 | −1.9096 |
| WIKI_SPHINGOLIPID_METABOLISM | −0.94592 | −1.6724 | 0.16285 |
| WIKI_KERATINIZATION | 6.3972 | −0.76908 | 6.2532 |
| WIKI_NRF2_PATHWAY | 1.7004 | 1.2883 | 1.7569 |
| WIKI_RESPIRATORY_ELECTRON_TRANSPORT_ATP_SYNTHESIS | 2.2177 | −1.3714 | 3.0362 |
| WIKI_ELECTRON_TRANSPORT_CHAIN_IN_MITOCHONDRIA | 2.7536 | −1.8586 | 3.3674 |

| Biological pathway | Niacinamide | Osmoter | Water |
|---|---|---|---|
| KEGG_SPHINGOLIPID_METABOLISM | −1.4272 | −0.96051 | −0.69307 |
| KEGG_CELL_CYCLE | −1.1781 | −1.7033 | −2.7927 |
| KEGG_NF_KAPPA_B_SIGNALING_PATHWAY | −0.79779 | −0.27732 | −1.0934 |
| KEGG_TNF_SIGNALING_PATHWAY | −1.6603 | −1.5848 | −1.2088 |
| KEGG_T_CELL_RECEPTOR_SIGNALING_PATHWAY | −0.515 | 0.070542 | 1.0492 |
| KEGG_SIGNALING_PATHWAYS_REGULATING_PLURIPOTENCY_OF_STEM_CELLS | −1.5828 | −1.6828 | −0.39481 |
| KEGG_CIRCADIAN_RHYTHM_MAMMAL | 0.15289 | −0.1074 | −0.06737 |
| KEGG_TIGHT_JUNCTION | 1.7592 | 1.2194 | 1.2529 |
| KEGG_GLYCOLYSIS_GLUCONEOGENESIS | 0.79763 | 1.2316 | 0.03586 |
| KEGG_FATTY_ACID_BIOSYNTHESIS | −1.579 | −1.605 | 1.2532 |
| KEGG_FATTY_ACID_DEGRADATION | −0.31192 | −0.49303 | −0.16766 |
| KEGG_TH1_AND_TH2_CELL_DIFFERENTIATION | −0.61061 | 0.049258 | 0.63177 |
| KEGG_APOPTOSIS | −0.67041 | −0.44056 | 0.67373 |
| KEGG_OXIDATIVE_PHOSPHORYLATION | 0.19588 | 1.166 | 0.31398 |
| KEGG_NUCLEOTIDE_EXCISION_REPAIR | −0.27434 | −0.33626 | −0.64844 |
| KEGG_BASE_EXCISION_REPAIR | 0.13435 | 0.3972 | 0.27445 |
| KEGG_P53_SIGNALING_PATHWAY | −0.041329 | −0.36427 | −0.55999 |
| KEGG_PROTEASOME | −1.4338 | −1.8176 | −1.6548 |
| KEGG_RETINOL_METABOLISM | 0.77971 | 0.71989 | 0.004292 |
| KEGG_WNT_SIGNALING_PATHWAY | −0.8093 | 0.17488 | 0.33941 |
| WIKI_COLLAGEN_BIOSYNTHESIS_AND_MODIFYING_ENZYMES | 1.0367 | 0.54031 | 1.4626 |
| WIKI_ASSEMBLY_OF_COLLAGEN_FIBRILS_AND_OTHER_MULTIMERIC_STRUCTURES | −0.48182 | −1.0256 | −0.64888 |
| WIKI_COLLAGEN_DEGRADATION | 0.74889 | −0.64717 | −0.018376 |
| WIKI_ACTIVATION_OF_MATRIX_METALLOPROTEINASES | 1.5758 | 0.66022 | 0.38361 |
| WIKI_DEGRADATION_OF_THE_EXTRACELLULAR_MATRIX | 0.025166 | −0.91266 | −1.0331 |
| WIKI_EXTRACELLULAR_MATRIX_ORGANIZATION | 0.27385 | −0.29879 | −0.14062 |
| WIKI_ELASTIC_FIBRE_FORMATION | −1.0086 | −1.113 | −0.55585 |
| WIKI_SPHINGOLIPID_METABOLISM | −0.14918 | 0.32587 | −0.71094 |
| WIKI_KERATINIZATION | 7.5403 | 8.6644 | 7.2273 |
| WIKI_NRF2_PATHWAY | 2.1898 | 1.6278 | 1.8759 |
| WIKI_RESPIRATORY_ELECTRON_TRANSPORT_ATP_SYNTHESIS | −0.75709 | −0.14926 | −0.78916 |
| WIKI_ELECTRON_TRANSPORT_CHAIN_IN_MITOCHONDRIA | −0.67575 | 0.23482 | −1.4404 |

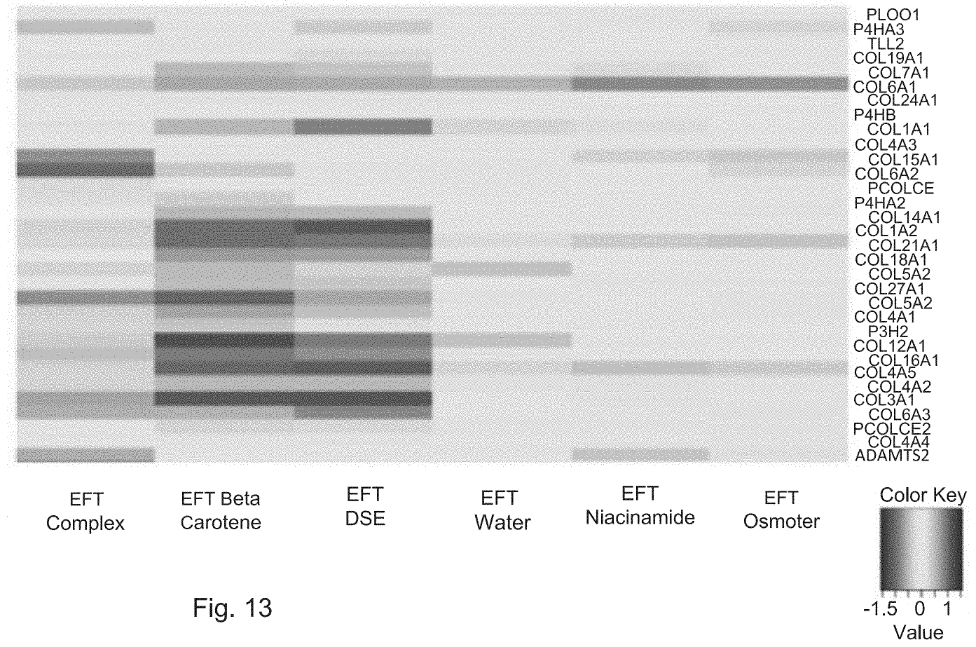

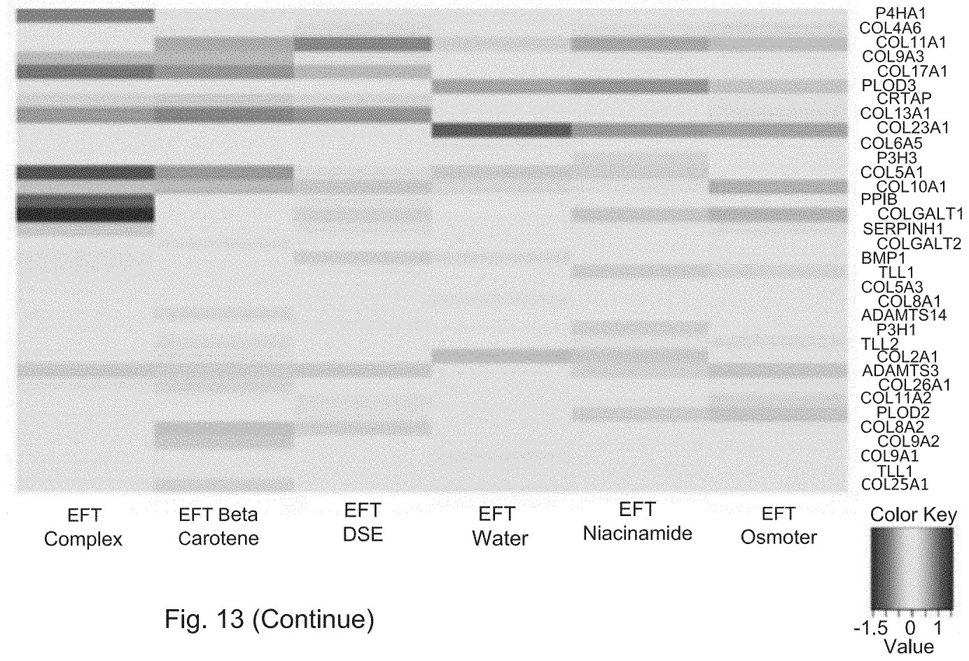

FIG. 13 illustrates the log FC heatmap for wiki collagen biosynthesis and modified enzymes pathway. It can be seen from FIG. 13 that genes in the upper middle contribute to the down-regulation of this pathway in EFT Beta Carotene and EFT DSE.

Similarly, the wiki keratinization pathway was checked and up-regulated genes in EFT_Niacinamide, EFT_Osmoter and EFT_Water were identified.

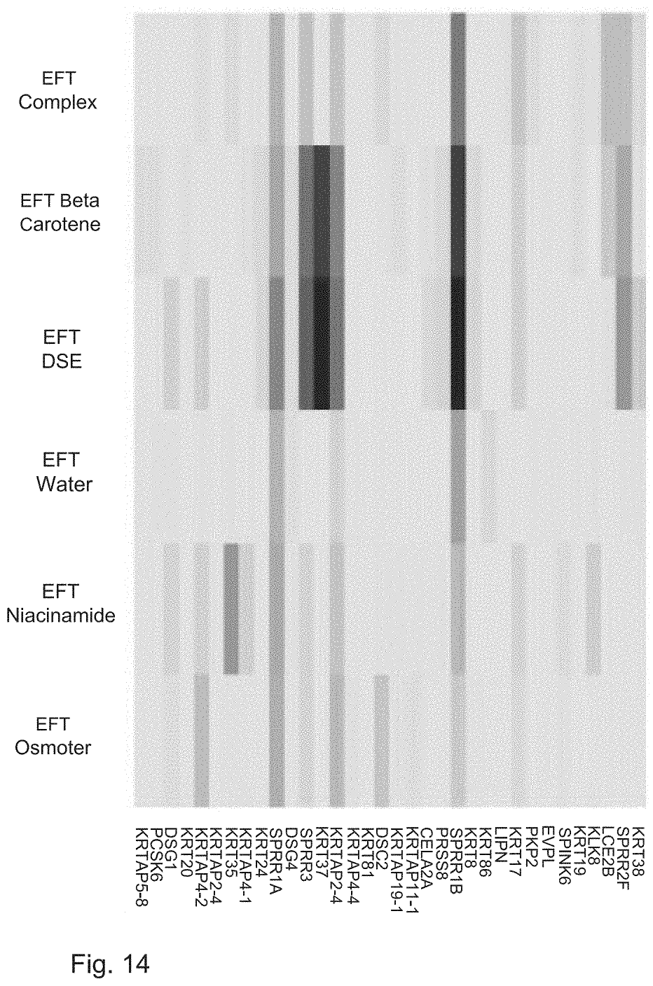

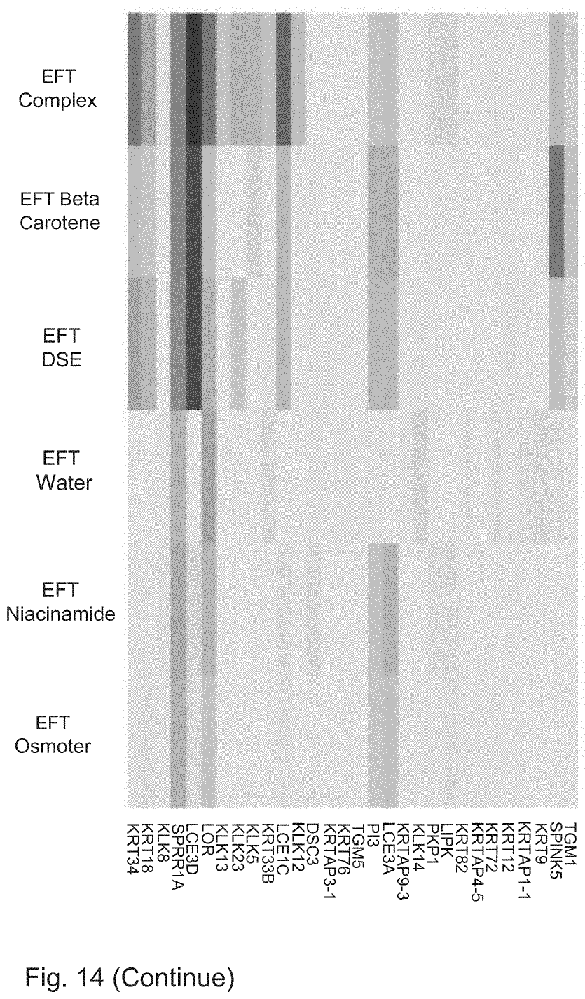

FIG. 14 illustrates the observed results of the analyzed wiki keratinization pathway. Specifically, FIG. 14 illustrates that genes KRT10, KRT1 are the most highly up-regulated (See arrow in the figure).

The generated data in Example 5 was used to analyze differences between the pRETINOL complex and each one of its individual ingredients.

The following biological pathways demonstrated unexpected effect in the gene expression when comparing the pRETINOL complex to each one of its ingredients:
KEGG Cell cycle.
KEGG Signaling pathways regulating pluripotency of stem cells.
KEGG Nucleotide excision repair.
KEGG P53 signaling pathway.
WIKI Assembly of collagen fibrils and other multimeric structures
WIKI Elastic fiber formation.
WIKI keratinization.

Table 8 details up and downregulation of genes in selected pathways where complex shows different expression than its individual components.

TABLE 8
Genes up and downregulation in selected pathways where complex shows different expression than its individual components.

| | | Beta | |
|---|---|---|---|
| Biological pathway | Complex | carotene | Osmoter |
| KEGG_CELL_CYCLE | + | − | − |
| KEGG_SIGNALING_PATHWAYS_REGULATING_PLURIPOTENCY_OF_STEM_CELLS | + | − | − |
| KEGG_NUCLEOTIDE_EXCISION_REPAIR | + | − | − |
| KEGG_P53_SIGNALING_PATHWAY | + | − | − |
| WIKI_ASSEMBLY_OF_COLLAGEN_FIBRILS_AND_OTHER_MULTIMERIC_STRUCTURES | + | − | − |
| WIKI_ELASTIC_FIBRE_FORMATION | + | − | − |
| WIKI_KERATINIZATION | NA* | + | + |

| Biological pathway | DSE | Niacinamide | Water |
|---|---|---|---|
| KEGG_CELL_CYCLE | − | − | − |
| KEGG_SIGNALING_PATHWAYS_REGULATING_PLURIPOTENCY_OF_STEM_CELLS | − | − | − |
| KEGG_NUCLEOTIDE_EXCISION_REPAIR | − | − | − |
| KEGG_P53_SIGNALING_PATHWAY | − | − | − |
| WIKI_ASSEMBLY_OF_COLLAGEN_FIBRILS_AND_OTHER_MULTIMERIC_STRUCTURES | − | − | + |
| WIKI_ELASTIC_FIBRE_FORMATION | − | − | − |
| WIKI_KERATINIZATION | + | + | + |

+ = up-regulated
− = down-regulated
*non-significant down-regulation, thus, regulation is considered as non-affected (NA)

### Example 6: Gene Expression for Relevant Biological Pathway—Microarray Results in Skin Equivalents—Comparative Data

In the present study the biological activity on gene expression of the pRETINOL complex was compared to the activity of a composition which was previously described in [13] and [14] and referred to herein as 3D complex. The activity was further compared to that observed with Retinol (5 μM of Retinol in double distilled water).

On each array, treated vs. untreated samples were compared. Each array was processed and the data from all arrays was normalized to get the average expression and log fold change (log FC) for each gene on each array.

An analysis of full human genome was performed.

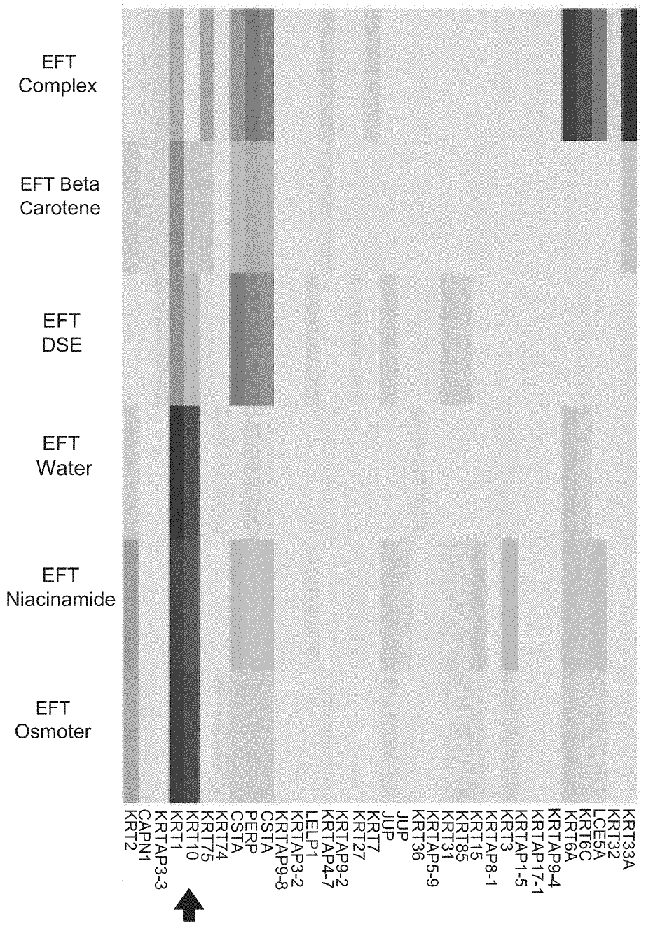

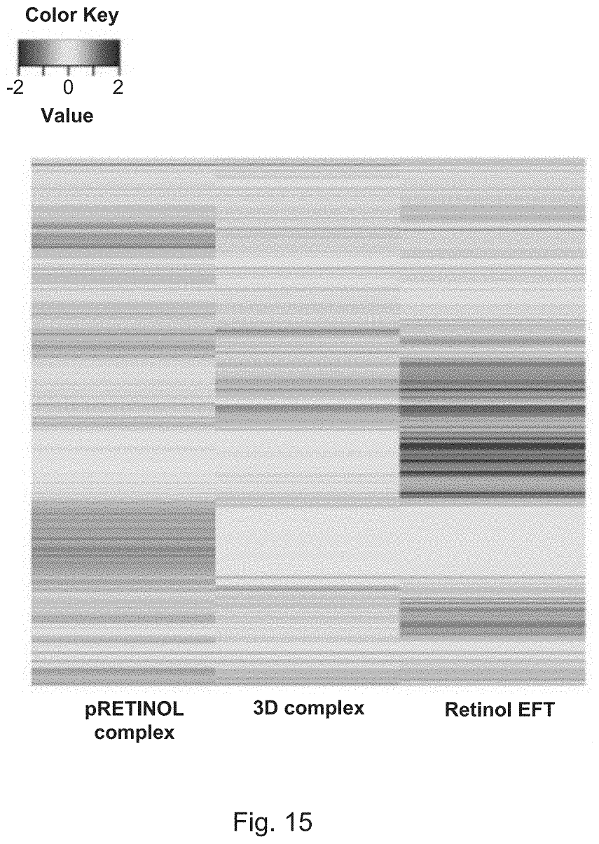

FIG. 15 illustrates the resulted Heatmap, Arrays Clustered by log FC of the present study. From the Heatmap of FIG. 15 it can be seen that the 3D complex shared some increased (red, colors not shown in grayscale figure) or decreased (blue, colors not shown in grayscale figure) genes with Retinol, but the changes measured in the 3D complex are typically smaller than those observed with the Retinol sample.

It can be further observed from the Heatmap of FIG. 15 that the pRETINOL complex presents a different pattern from the other tested samples (i.e., 3D complex and Retinol).

It can also be observed from the Heatmap of FIG. 15 that in the middle of the Heatmap, a similarity of up-regulated genes (red, colors not shown in grayscale figure) is observed i.e., some genes upregulation is common among all of the tested samples.

Further to the above analysis, similar to the study presented in Example 5, major skin related pathways were selected from KEGG and wiki pathways list and the p-value and z-score were calculated for these pathways.

The results for the pRETINOL complex significant biological pathways gene expression is illustrated in Table 9 which represents numeric data of FDR, p-values, z-scores, number of genes tested, number of down regulated genes and number of upregulated genes.

TABLE 9
The results for pRETINOL complex significant biological pathways gene expression.

| | Genes | Genes | | | | Genes |
|---|---|---|---|---|---|---|
| Biological pathway | (down) | (up) | FDR | p-value | z-score | (total) |
| KEGG_SPHINGOLIPID_METABOLISM | 21 | 14 | 0.23377 | 0.041958 | −1.8128 | 35 |
| KEGG_NF_KAPPA_B_SIGNALING_PATHWAY | 50 | 38 | 0.13247 | 0.016983 | −2.2215 | 88 |
| KEGG_TNF_SIGNALING_PATHWAY | 68 | 39 | 0.019481 | 0.000999 | −4.7227 | 107 |
| KEGG_APOPTOSIS | 53 | 32 | 0.087662 | 0.008991 | −2.5587 | 85 |
| KEGG_BASE_EXCISION_REPAIR | 13 | 19 | 0.54545 | 0.027972 | 1.8866 | 32 |
| WIKI_KERATINIZATION | 48 | 89 | 0.038961 | 0.000999 | 4.936 | 137 |
| KEGG_NOD-LIKE_RECEPTOR | 99 | 69 | 0.019481 | 0.000999 | −4.4693 | 168 |
| SIGNALING PATHWAY | | | | | | |
| KEGG_YERSINIA_INFECTION | 68 | 46 | 0.025974 | 0.001998 | −3.2429 | 114 |

The data presented in Table 9 illustrate a significant effect of the pRETINOL complex which is reflected in the observed relatively low p-values and a relatively larger absolute z-score value.

The results of the biological relevant pathways presented in Table 9 above were compared to those observed with the 3D complex and the Retinol sample. The results are summarized in Table 10 which represents numeric data of the observed z-scores values.

TABLE 10
Comparative z-scores values of significant biological pathways gene expression.

| | | 3D | pRETINOL |
|---|---|---|---|
| Biological pathway | Retinol | complex | complex |
| KEGG_SPHINGOLIPID_METABOLISM | −0.6417 | −0.56211 | −1.8128 |
| KEGG_NF_KAPPA_B_SIGNALING_PATHWAY | −0.46727 | 0.33001 | −2.2215 |
| KEGG_TNF_SIGNALING_PATHWAY | −1.1097 | −1.802 | −4.7227 |
| KEGG_APOPTOSIS | 1.0758 | 0.021045 | −2.5587 |
| KEGG_BASE_EXCISION_REPAIR | −0.28178 | 1.5498 | 1.8866 |
| WIKI_KERATINIZATION | 10.548 | 7.1478 | 4.936 |
| KEGG_NOD-LIKE_RECEPTOR | −0.35018 | −1.4703 | −4.4693 |
| SIGNALING PATHWAY | | | |
| KEGG_YERSINIA_INFECTION | 1.5054 | −0.0048953 | −3.2429 |

In the data presented in Table 10, a positive z-score value means that the pathway tends to be up-regulated and a negative z-score value means that the pathway tends to be down-regulated.

The bold marked z-scores values in Table 10 are those that were found statistically significant. As illustrated in Table 10, the pRETINOL complex of the present invention illustrated significant z-score values.

Further, the relatively larger absolute z-score values observed with the pRETINOL complex compared to those observed with the 3D complex and the Retinol sample indicate a significant effect of the pRETINOL complex of the present invention. This observed effect also indicates on the efficiency of the complex of the invention compared to the 3D complex. Further, the effect indicates safe activity of the complex of the invention e.g., without the side effects associate with direct application of retinol onto the skin.

The above results also indicate that the pRETINOL complex of the present invention can have retinol like activity by affecting of keratinization pathway. In addition, the results indicate that the pRETIOL complex is safer compared to retinol as it reduced gene expression related to pathways of inflammation (e.g., KEGG_YERSINIA_INFECTION and KEGG_NOD-LIKE_RECEPTOR SIGNALING PATHWAYS which are inflammasome related) and apoptosis (e.g., KEGG_APOPTOSIS).

### Example 7: β-Carotene and Niacinamide Quantification—Comparative Data

The content of β-Carotene and Niacinamide in one complex of the present invention (pRETINOL complex) was determined and compared to the content of the β-Carotene and Niacinamide in the prior composition 3D complex which was previously described in [13] and [14], said prior composition comprises Dead Sea water, Dunaliella Salina extract, Ziziphus jujuba extract and Trigonella Foenum extract.

A. β-Carotene Quantification

The content of β-Carotene in the complexes was determined using the following assay:

A β-Carotene calibration curve was prepared utilizing a β-Carotene standard (potency of 93%). A calibration curve of β-Carotene diluted in ethanol at the following concentration was prepared: 1.0, 0.5, 0.25, 0.125, 0.063, 0.031 and 0.016 μg/ml. Ethanol was use as a blank.

Samples Preparation:

β-Carotene Powder Raw Material

500 mg of β-Carotene powder 10% CWS/s raw material was diluted in 50 ml volumetric flask and 1 ml of water was added for mixing and the solution was left to stand for 5 min. The flask was diluted with ethanol to volume of 50 ml [1 mg/ml β-Carotene]. The resulted diluted sample was further diluted (×100) with ethanol and the absorbance was measured.

Plant Extract

1 ml of each plant extract was diluted to a final volume of 10 ml with ethanol. The sample was centrifuge for 10 min at 3000 rpm. The supernatant was measure for absorbance.

Standards and samples were measured for absorbance at 455 nm (measured at maximum) in a 96 wells plate.

The final concentration of β-Carotene in the pRETINOL complex and the 3D complex (in ppm units) are summarized in Table 11.

TABLE 11
Concentration of β-Carotene for pRetinol and 3D complexes (in ppm units).

| | | | | β-Carotene |
|---|---|---|---|---|
| | β-Carotene | β-Carotene | | concentration |
| | in original | in original | | in the final |
| | extraction | extraction | % in | cosmetic cream |
| Samples Name | (ug/ml) | (ppm) | complex | (ppm) |
| β-Carotene powder | 113000 | 113000 | | |
| Dunalliela Salina water based | 6.0 | 6.0 | 3.0 | 0.18 |
| extract (used in the pRetinol | | | | |
| complex) | | | | |
| β-Carotene content calculated | | | | 15.6* |
| from powder (used in the | | | | |
| pRetinol complex) | | | | |
| Final concentration of | | | | 15.78 |
| β-Carotene in the | | | | |
| pRetinol complex | | | | |
| Dunalliela Salina water based | 6.0 | 6.0 | 0.5 | 0.03 |
| extract (used in the 3D complex) | | | | |
| Ziziphus jujuba extract (used | 1.6 | 1.6 | 0.75 | 0.012 |
| in the 3D complex) | | | | |
| Trigonella Foenum extract | 0.3 | 0.3 | 0.2 | 0.0006 |
| (used in the 3D complex) | | | | |
| Final concentration of | | | | 0.0426 |
| β-Carotene in the 3D complex | | | | |

*β-Carotene content calculated from powder (used in the pRetinol complex): a 0.5 gr of 10% β-Carotene powder were diluted with DDW at a 50 ml volumetric flask, to obtain a 0.1% of β-Carotene. A 156 μl of this solution were transferred to 10 ml of pRetinol complex solution to obtain a final concentration of 15.6 ppm β-Carotene.

The above data indicate that the final concentration of the β-Carotene in the pRetinol complex of the present invention was 15.78 ppm (i.e., 0.18 ppm+15.6 ppm, the sum of the β-Carotene from the powder and the Dunalliela Salina extract) while the final concentration of the β-Carotene in the 3D complex was 0.0426 ppm (i.e., 0.03 ppm+0.0006+0.0426 ppm, the sum of the β-Carotene from the Dunalliela Salina extract, the Ziziphus jujuba extract and the Trigonella Foenum extract) which is 370 times lower than the content of the β-Carotene in the pRetinol complex of the present invention. It is noted that this observed difference in the content of the β-Carotene is provided in comparison with the final cosmetic cream of [13] and [14] i.e., 3D complex, which is intended for topical application. A 20 times more concentrated composition is described in [13] and [14], said concentrated composition, which is eventually diluted for topical use, thus has a final concentration of β-Carotene of 0.852 ppm which is 18.5 times lower than the content of the β-Carotene in the pRetinol complex of the present invention.

B. Niacinamide Quantification

The content of Niacinamide in the complexes was determined utilizing a 4.6×250 mm C18 column with a mobile phase being 0.1M sodium acetate solution adjusted to pH 5.4 with acetic acid. A small amount of methanol (up to 1%) was added to the mobile phase for improved resolution. The flow rate was 1.0 ml/min. UV detection at 254 nm was performed.

The final concentration of Niacinamide in the pRETINOL complex and the 3D complex (in w/w s) are summarized in Table 12.

TABLE 12
Concentration of Niacinamide for pRetinol and 3D complexes.

| | | | Niacinamide | |
|---|---|---|---|---|
| | Niacinamide | | (% w/w) in | Niacinamide |
| | in original | | tested sample | (% w/w) in |
| | extraction | % in | (raw | final cosmetic |
| Samples Name | (mg/Kg) | complex | extract/complex) | cream |
| pRetinol Complex | 1047 | 100 | 0.1047 | 0.1047 |
| Final concentration of | | | | 0.1047 |
| Niacinamide in the | | | | |
| pRetinol complex | | | | |
| Dunalliela Salina water base | 602.7 | 0.5 | 0.0603 | 0.00030 |
| extract (used in the 3D | | | | |
| complex) | | | | |
| Ziziphus jujuba extract | <10 | 0.75 | N/A | N/A |
| (used in the 3D complex) | | | | |
| Trigonella Foenum extract | 49.92 | 0.2 | 0.005 | 0.000 |
| (used in the 3D complex) | | | | |
| Final concentration of | | | | 0.0003 |
| Niacinamide in 3D | | | | |
| complex | | | | |

The above data indicate that the final concentration of the Niacinamide in the pRetinol complex of the present invention was 0.1047% (w/w) while the final concentration of the Niacinamide in the 3D complex was 0.0003% (w/w) (the sum of the Niacinamide from the Dunalliela Salina extract, the Ziziphus jujuba extract and the Trigonella Foenum extract, with the Niacinamide content in each of the Ziziphus jujuba extract and the Trigonella Foenum being negligible) which is 349 times lower than the content of the Niacinamide in the pRetinol complex of the present invention.

The above quantification data indicate that the compositions of the present invention comprises both β-carotene and Niacinamide. The β-carotene and Niacinamide are present in the compositions of the present invention in an amount effective to induce the various effects of the compositions of the invention (e.g., therapeutic and/or a cosmetic) as disclosed herein above and below. Without wishing to be bound by theory, these effects may be associated with intercellular production of Vitamin A upon topical application.

It is clear that the amounts of each of the β-carotene and the Niacinamide in the prior 3D complex are amounts that are non-effective according to the methods and/or uses of the present inventions and are therefore excluded from present invention.

### Example 8: Study the Effect of the pRETINOL Complex on the Production of Hyaluronic Acid in Human Primary Fibroblasts and Comparing to the Effect of Retinol

The effect of the pRETINOL complex on the production of hyaluronic Acid in human primary fibroblasts was studied and compared to the effect of Retinol.

Hyaluronic Acid Production Assay:

The human primary fibroblast cell culture was grown at an 80% confluence in Dulbecco's Modified Eagle's High glucose (Hg) medium (DMEM) culture medium containing 10% fetal bovine serum, 100 U/ml penicillin, and 100 U/ml streptomycin. The cells were cultivated in 6 wells plates.

The pRetinol complex was added at a final concentrations of. Osmoter 0.2%, Dunaliella salina extract 1.2%, Niacinimide 0.04% and 6.24 ppm of β-Carotene i.e., 2.5 times dilution (the dilution was necessary in view of the human primary fibroblast cells culture being more sensitive than skin full organ culture). Retinol was added to the medium at a final concentration of 1.0 μM.

The cells were incubated at 37° C., 5% CO₂ at a 100% RH for 48 hours. After incubation the cells were extracted using RIPA buffer collected to a blending Eppendorf and ground using stain steal beads and a bullet blender for 5 min. The samples were centrifuged for 15 min at 5000 rpm and the supernatant was collected.

BCA Protein concentration assay were performed on all the samples. The protein level for all samples was dilated to reach a concentration of 0.1 ng/ml of protein, diluted with PBS. Samples contain a protein concentration of 0.1 ng/ml were tested for hyaluronic ELISA assay kit (R&D systems, ELISA kit). Results were obtained as ng/ml of hyaluronic acid per 1 μg of protein.

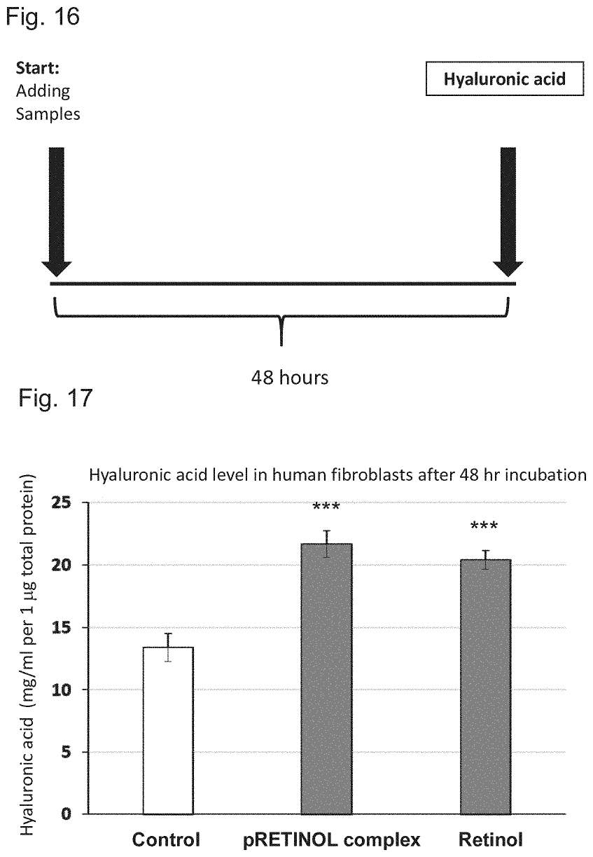

FIG. 16 illustrates a flow diagram of a human primary fibroblast cells assay in which said cells were exposed to the pRETINOL complex (2.5 times diluted in the culture media) and to the retinol sample (1.0 μM in the culture media) followed by determining the hyaluronic acid content after exposure for 48 hours.

FIG. 17 illustrates the hyaluronic acid observed levels in human fibroblast after 48 hr incubation with the pRETINOL complex and the Retinol sample. The observed hyaluronic acid exposed to a control sample (culture medium) are also illustrated in the figure.

The results in FIG. 17 illustrate that both the pRETINOL complex and the retinol sample induce the production of hyaluronic acid in human primary fibroblasts to a great extent compare to the control sample. The results further illustrate that the effect of the pRETINOL complex on the hyaluronic acid production is greater than the effect of the retinol sample.

Without wishing to be bound by theory, the inventors believe that pRETINOL complex is advantageous over retinol at least in view of the fact that application of retinol onto the skin is associated with skin irritation. Hence, the composition of the present invention beneficially induced the production of hyaluronic acid when applied onto the skin, without the side effects associated with direct application of retinol onto the skin.

### Illustrative Embodiments

The following embodiments are illustrative and not intended to limit the claimed subject matter.

Embodiment 1 A composition comprising at least one Dead Sea extract, at least one extract of Dunaliella alga selected from one or both of Dunaliella Salina extract and Dunaliella Bardawill extract, β-carotene and Niacinamide, wherein the concentration of the β-carotene in said composition is at least about 1 ppm and the concentration of the Niacinamide in said composition is at least about 0.02% w/w.

Embodiment 2 The composition according to embodiment 1, wherein said Dead Sea extract is a mixture of natural materials obtained from the waters of the Dead Sea, the mud surrounding the Dead Sea and/or the soil bed of the Dead Sea.

Embodiment 3 The composition according to embodiment 1, wherein said Dead Sea extract is the saline waters obtained from the Dead Sea.

Embodiment 4 The composition according to embodiment 3, wherein the Dead Sea water has a specific density of 1.25-1.35 g/ml, pH of 4.6-5.6 (at 25° C.), and less than 100 cfu/g of non-pathogenic microbes.

Embodiment 5 The composition according to any one of embodiments 2 to 4, wherein the Dead Sea water comprises Ca⁺², Cl⁻, Mg⁺², Na⁺, K⁺ and Br⁻.

Embodiment 6 The composition according to embodiment 1, wherein said Dead Sea extract is an aqueous solution simulating the salts and minerals content of the Dead Sea water.

Embodiment 7 The composition according to any one of embodiments 1 to 6, wherein said Dead Sea extract is the commercially available product Maris Sal.

Embodiment 8 The composition according to any one of embodiments 1 to 7, wherein said Dead Sea extract is present in said composition at a concentration of between about 0.05% w/w to about 5.0% w/w e.g., between about 0.25% w/w to about 2.5% w/w.

Embodiment 9 The composition according to embodiment 8, wherein the concentration of said Dead Sea extract in said composition is about 0.5% w/w.

Embodiment 10 The composition according to embodiment 8, wherein said Dead Sea extract is Dead Sea water and wherein said Dead Sea water is present in said composition at a concentration of between about 0.05% w/w to about 5.0% w/w e.g., between about 0.25% w/w to about 2.5% w/w.

Embodiment 11 The composition according to embodiment 10, wherein the concentration of said Dead Sea water in said composition is about 0.5% w/w.

Embodiment 12 The composition according to any one of embodiments 1 to 11, wherein said at least one extract of Dunaliella alga is an extract of Dunaliella Salina e.g., obtained from a Dunaliella Salina originated from the habitats of the Dead Sea.

Embodiment 13 The composition according to any one of embodiments 1 to 12, wherein said at least one extract of the Dunaliella alga is an hydrophilic extract.

Embodiment 14 The composition according to any one of embodiments 1 to 13, wherein said at least one extract of the Dunaliella alga is an aqueous extract.

Embodiment 15 The composition according to any one of embodiments 1 to 14, wherein the Dunaliella alga extract is present in said composition at a concentration of between about 0.1% w/w to about 5.0% w/w e.g., between about 0.5% w/w to about 3.0% w/w.

Embodiment 16 The composition according to embodiment 15, wherein the Dunaliella alga extract is Dunaliella Salina extract and wherein the concentration thereof in said composition is about 3.0% w/w.

Embodiment 17 The composition according to any one of embodiments 1 to 16, wherein the β-carotene is a non-synthetic β-carotene.

Embodiment 18 The composition according to any one of embodiments 1 to 17, wherein the β-carotene is from a source different from the Dunaliella alga.

Embodiment 19 The composition according to any one of embodiments 1 to 16, wherein the β-carotene is a synthetic β-carotene.

Embodiment 20 The composition according to any one of embodiments 1 to 19, wherein the concentration of the β-carotene in said composition is between about 5 ppm to about 500 ppm e.g., between about 5 ppm to about 50 ppm.

Embodiment 21 The composition according to any one of embodiments 1 to 20, wherein the concentration of the β-carotene in said composition is 15.6 ppm or 15.78 ppm.

Embodiment 22 The composition according to any one of embodiments 1 to 21, wherein the Niacinamide is non-synthetic.

Embodiment 23 The composition according to any one of embodiments 1 to 23, wherein the Niacinamide is from a source different from a plant extract.

Embodiment 24 The composition according to embodiment 23, wherein said plant extract is one or both of Zizyphus and Trigonella foenum plant extract.

Embodiment 25 The composition according to embodiment 24, wherein said plant extract is Zizyphus plant extract.

Embodiment 26 The composition according to embodiment 24, wherein said plant extract is Trigonella foenum plant extract.

Embodiment 27 The composition according to any one of embodiments 1 to 26, wherein the Niacinamide is synthetic.

Embodiment 28 The composition according to any one of embodiments 1 to 27, wherein the concentration of the Niacinamide in said composition is between about 0.02% w/w to about 5.0% w/w e.g., between about 0.05% w/w to about 0.2% w/w.

Embodiment 29 The composition according to embodiment 28, wherein the concentration of the Niacinamide in said composition is 0.10% w/w.

Embodiment 30 The composition according to any one of embodiments 1 to 29, wherein said composition comprises about 0.25% w/w to about 2.5% w/w Dead Sea extract, about 0.5% w/w to about 3.0% w/w Dunaliella Salina extract, about 5 ppm to about 50 ppm β-carotene and about 0.05% w/w to about 0.2% w/w Niacinamide.

Embodiment 31 The composition according to embodiment 30, wherein said composition comprises about 0.5% w/w Dead Sea extract, about 3.0% w/w Dunaliella Salina extract, about 15.6 ppm β-carotene and about 0.1% w/w Niacinamide.

Embodiment 32 The composition according to embodiment 31, wherein said composition comprises about 0.5% w/w Dead Sea water, about 3.0% w/w Dunaliella Salina extract, about 15.6 ppm β-carotene and about 0.1% w/w Niacinamide.

Embodiment 33 A composition for enriching a skin cell with at least one Vitamin A (e.g., retinol), the composition comprising at least one Dead Sea extract, at least one Dunaliella alga extract selected from one or both of Dunaliella Salina extract and Dunaliella Bardawill extract and a combination of β-carotene and Niacinamide, wherein the combination comprises an amount of each of β-carotene and Niacinamide sufficient to induce intercellular production of at least one Vitamin A (e.g., retinol) upon application of said composition onto at least a region of a skin of a subject, thereby enriching a skin cell with at least one Vitamin A.

Embodiment 34 The composition according to embodiment 33 being a composition according to any one of embodiments 1 to 32.

Embodiment 35 The composition according to embodiment 33, wherein said composition comprises about 0.25% w/w to about 2.5% w/w Dead Sea extract, about 0.5% w/w to about 3.0% w/w Dunaliella Salina extract, about 5 ppm to about 50 ppm β-carotene and about 0.05% w/w to about 0.2% w/w Niacinamide.

Embodiment 36 The composition according to embodiment 35, wherein said composition comprises about 0.5% Dead Sea extract, about 3.0% w/w Dunaliella Salina extract, about 15.6 ppm β-carotene and about 0.1% w/w Niacinamide.

Embodiment 37 The composition according to any one of embodiments 1 to 36, being a composition selected from a skin-care and pharmaceutical composition.

Embodiment 38 The composition according to embodiment 37, wherein said composition is for topical application.

Embodiment 39 The composition according to embodiment 38, being a synergistic composition.

Embodiment 40 The composition according to any one of embodiments 1 to 39, being in the form selected from a lotion, an ointment, a gel, a mask, a toner, an essence, a cream, a water in oil or oil in water emulsion, a shampoo, a moisturizer, a sunscreen, a cream, a stick, a spray, an aerosol, foam, a paste, a mousse, a solid, semi-solid, or a liquid make-up, a foundation, and an eye make-up.

Embodiment 41 The composition according to any one of embodiments 1 to 40, further comprising at least one additive selected from a diluent, a preservative, an abrasive, an anticaking agent, an antistatic agent, a binder, a buffer, a dispersant, an emollient, an emulsifier, a co-emulsifiers, a fiberous material, a film forming agent, a fixative, a foaming agent, a foam stabilizer, a foam booster, a gellant, a lubricant, a moisture barrier agent, a plasticizer, a preservative, a propellant, a stabilizer a surfactant, a suspending agent, a thickener, a wetting agent, and a liquefier.

Embodiment 42 The composition according to any one of embodiments 1 to 41, further comprising at least one additive selected from an anti-acne agent, an anti-aging agent, an antibacterial agent, an anti-cellulites agent, an antidandruff agent, an antifungal agent, an anti-inflammatory agent, an anti-irritation agent, an antimicrobial agent, an antioxidant agent, an antiperspirant agent, an antiseptic agent, a cell stimulant, a cleansing agent, a conditioner, a deodorant, a depilatory, a detergent, an enzyme, an essential oil, an exfoliant, a fungicide, a glosser, hair conditioner, hair set resin, hair sheen agent, hair waving agent, a humectants, a moisturizer, an ointment base, a perfume, a protein, a skin calming agent, a skin cleanser, a skin conditioner, a skin healing agent, a skin lightening agent, a skin protectant, a skin smoothing agent, a skin softening agent, a skin soothing agent, a sunscreen agent, a tanning accelerator, vitamins, a colorant, and a flavoring agent.

Embodiment 43 The composition according to any one of embodiments 1 to 42 for use in the preparation of a cosmetic/skin-care or pharmaceutical composition.

Embodiment 44 The composition according to embodiment 43, wherein said composition is for one or more of protecting and/or improving the state of the skin, and preventing and/or treating imperfections of the skin of a subject.

Embodiment 45 The composition according to embodiment 44, wherein the composition is for treating rings under the eye, symptoms of aging, protecting the skin, increasing the detoxification of xenobiotics, intervening on pigmentation level, inhibiting melanogenesis, protecting the body against pollution, stimulating the detoxification systems, stimulating hair and body hair growth, modulating DHT levels, intervening on adipocytes, and/or promoting lipolysis.

Embodiment 46 The composition according to embodiment 44, wherein said composition is for treating or preventing at least one disease or disorder of the skin.

Embodiment 47 The composition according to embodiment 46, wherein said disease or disorder of the skin is a secondary condition, being related to an existing condition.

Embodiment 48 The composition according to embodiment 47, wherein said secondary condition is inflammation.

Embodiment 49 A lotion, an ointment, a gel, a mask, a toner, an essence, a shampoo, a moisturizer, a sunscreen, a cream, a stick, a spray, an aerosol, foam, a paste, a mousse, a solid, semi-solid, or a liquid make-up, a foundation, or an eye make-up comprising a composition according to any one of embodiments 1 to 48.

Embodiment 50 The composition according to any one of embodiments 1 to 48 for use in the treatment and/or prevention of one or more disease or disorder, wherein said disease or disorder is associated with and/or is mediated by and/or is affected by and/or is related to one or more of biological pathways beings selected from cell cycle (KEGG, Kyoto Encyclopedia of Genes and Genomes); signaling pathways regulating pluripotency of stem cells (KEGG); nucleotide excision repair (KEGG); P53 signaling pathway (KEGG); assembly of collagen fibrils and other multimeric structures (WIKI); Elastic fiber formation (WIKI); keratinization (WIKI); or any combination thereof.

Embodiment 51 The composition according to any one of embodiments 1 to 48 for use in a method of one or more of protecting and/or improving the state of the skin of a subject, and preventing and/or treating imperfections of the skin of a subject in need thereof, said method comprising topically administering a composition according to any one of embodiments 1 to 48 onto the skin of said subject.

Embodiment 52 The composition according to embodiment 51, wherein the imperfections of the skin of a subject is due to deficiency of retinol.

Embodiment 53 The composition according to embodiment 51 or 52, wherein the protecting and/or improving the state of the skin of a subject, and preventing and/or treating imperfections of the skin of a subject in need thereof is mediated by at least one Vitamin A e.g., retinol.

Embodiment 54 The composition according to any one of embodiments 51 to 53, for treating rings under the eye, symptoms of aging, protecting the skin, increasing the detoxification of xenobiotics, intervening on pigmentation level, inhibiting melanogenesis, protecting the body against pollution, stimulating the detoxification systems, stimulating hair and body hair growth, modulating DHT levels, intervening on adipocytes, and/or promoting lipolysis.

Embodiment 55 The composition according to any one of embodiments 1 to 54 for use in a method for treating or preventing a disease or disorder of the skin of a subject, said method comprising administering to a subject in need thereof a composition according to any one of embodiments 1 to 54.

Embodiment 56 The composition according to embodiment 55, wherein said disease or disorder of the skin is a secondary condition, being related to an existing condition.

Embodiment 57 A method for treating and/or preventing one or more disease or disorder, the method comprises administration of the composition according to any one of embodiments 1 to 56 to a subject in need thereof, wherein said disease or disorder is associated with and/or is mediated by and/or is affected by and/or is related to one or more of biological pathways beings selected from cell cycle (KEGG, Kyoto Encyclopedia of Genes and Genomes); signaling pathways regulating pluripotency of stem cells (KEGG); nucleotide excision repair (KEGG); P53 signaling pathway (KEGG); assembly of collagen fibrils and other multimeric structures (WIKI); Elastic fiber formation (WIKI); keratinization (WIKI); or any combination thereof.

Embodiment 58 A method of enriching a skin cell with at least one Vitamin A e.g., retinol, the method comprising:
applying onto at least a region of a skin of a subject a composition comprising at least one Dead Sea extract, at least one Dunaliella alga extract selected from one or both of Dunaliella Salina extract and Dunaliella Bardawill extract and a combination of β-carotene and Niacinamide, wherein the combination comprises an amount of each of 0-carotene and Niacinamide sufficient to induce intercellular production of Vitamin A e.g., retinol, thereby enriching the skin cell with retinol.

Embodiment 59 The method according to embodiment 58, wherein said composition being a composition according to any one of embodiments 1 to 56.

Embodiment 60 The method according to embodiment 59, wherein said composition comprises about 0.25% w/w to about 2.5% w/w Dead Sea extract, about 0.5% w/w to about 3.0% w/w Dunaliella Salina extract, about 5 ppm to about 50 ppm β-carotene and about 0.05% w/w to about 0.2% w/w Niacinamide.

Embodiment 61 The method according to embodiment 60, wherein said composition comprises about 0.5% Dead Sea extract, about 3.0% w/w Dunaliella Salina extract, about 15.6 ppm β-carotene and about 0.1% w/w Niacinamide.

Embodiment 1A A composition comprising at least one Dead Sea extract, β-carotene and Niacinamide, wherein the concentration of the β-carotene in said composition is at least about 1 ppm and the concentration of the Niacinamide in said composition is at least about 0.02% w/w.

Embodiment 2A The composition according to embodiment 1A, wherein said composition further comprises at least one extract of Dunaliella alga.

Embodiment 3A The composition according to embodiment 1A or 2A, wherein said at least one extract of Dunaliella alga is selected from Dunaliella Salina extract, Dunaliella Bardawill extract or a combination thereof.

Embodiment 4A The composition according to any one of embodiments 1A to 3A, wherein said composition comprises at least one Dead Sea extract, at least one extract of Dunaliella alga, β-carotene and Niacinamide, wherein the concentration of the β-carotene in said composition is at least about 1 ppm and the concentration of the Niacinamide in said composition is at least about 0.02% w/w.

Embodiment 5A The composition according to any one of embodiments 1A to 4A, wherein said Dead Sea extract is a mixture of natural materials obtained from the waters of the Dead Sea, the mud surrounding the Dead Sea and/or the soil bed of the Dead Sea.

Embodiment 6A The composition according to any one of embodiments 1A to 4A, wherein said Dead Sea extract is the saline waters obtained from the Dead Sea.

Embodiment 7A The composition according to embodiment 6A, wherein the Dead Sea water has a specific density of 1.25-1.35 g/ml, pH of 4.6-5.6 (at 25° C.), and less than 100 cfu/g of non-pathogenic microbes.

Embodiment 8A The composition according to any one of embodiments 5A to 7a, wherein the Dead Sea water comprises Ca⁺², Cl⁻, Mg⁺², Nat, K⁺ and Br⁻.

Embodiment 9A The composition according to any one of embodiments 1A to 8A, wherein said Dead Sea extract is an aqueous solution simulating the salts and minerals content of the Dead Sea water.

Embodiment 10A The composition according to any one of embodiments 1A to 9A, wherein said Dead Sea extract is the commercially available product Maris Sal.

Embodiment 11A The composition according to any one of embodiments 1A to 10A, wherein said Dead Sea extract is present in said composition at a concentration of between about 0.05% w/w to about 5.0% w/w.

Embodiment 12A The composition according to embodiment 11A, wherein the concentration of said Dead Sea extract in said composition is about 0.20% w/w or about 0.50% w/w.

Embodiment 13A The composition according to embodiment 11A, wherein said Dead Sea extract is Dead Sea water and wherein said Dead Sea water is present in said composition at a concentration of between about 0.05% w/w to about 5.0% w/w.

Embodiment 14A The composition according to embodiment 13A, wherein the concentration of said Dead Sea water in said composition is about 0.20% w/w or about 0.50% w/w.

Embodiment 15A The composition according to any one of embodiments 2A to 14A, wherein said at least one extract of Dunaliella alga is an extract of Dunaliella Salina obtained from a Dunaliella Salina originated from the habitats of the Dead Sea.

Embodiment 16A The composition according to any one of embodiments 2A to 15A, wherein said at least one extract of the Dunaliella alga is an hydrophilic extract.

Embodiment 17A The composition according to any one of embodiments 2A to 16A, wherein said at least one extract of the Dunaliella alga is an aqueous extract.

Embodiment 18A The composition according to any one of embodiments 2A to 17A, wherein the Dunaliella alga extract is present in said composition at a concentration of between about 0.1% w/w to about 5.0% w/w.

Embodiment 19A The composition according to embodiment 18A, wherein the Dunaliella alga extract is present in said composition at a concentration of between about 0.5% w/w to about 3.0% w/w.

Embodiment 20A The composition according to embodiment 18A or 19A, wherein the Dunaliella alga extract is Dunaliella Salina extract and wherein the concentration thereof in said composition is about 1.2% or about 3.0% w/w.

Embodiment 21A The composition according to any one of embodiments 1A to 20A, wherein the β-carotene is a non-synthetic β-carotene.

Embodiment 22A The composition according to any one of embodiments 1A to 21A, wherein the β-carotene is from a source different from a Dunaliella alga.

Embodiment 23A The composition according to any one of embodiments 1A to 20A, wherein the β-carotene is a synthetic β-carotene.

Embodiment 24A The composition according to any one of embodiments 1A to 23A, wherein the concentration of the β-carotene in said composition is between about 1 ppm to about 500 ppm.

Embodiment 25A The composition according to any one of embodiments 1A to 23A, wherein the concentration of the β-carotene in said composition is between about 1 ppm to about 50 ppm.

Embodiment 26A The composition according to any one of embodiments 1A to 23A, wherein the concentration of the β-carotene in said composition is between about 5 ppm to about 50 ppm.

Embodiment 27A The composition according to any one of embodiments 1A to 26A, wherein the concentration of the β-carotene in said composition is 15.78 ppm.

Embodiment 28A The composition according to any one of embodiments 1A to 26A, wherein the concentration of the β-carotene in said composition is 6.24 ppm.

Embodiment 29A The composition according to any one of embodiments 1A to 28A, wherein the Niacinamide is non-synthetic.

Embodiment 30A The composition according to any one of embodiments 1A to 29A, wherein the Niacinamide is from a source different from a plant extract.

Embodiment 31A The composition according to embodiment 30A, wherein said plant extract is one or both of Zizyphus and Trigonella foenum plant extract.

Embodiment 32A The composition according to embodiment 31A, wherein said plant extract is Zizyphus plant extract.

Embodiment 33A The composition according to embodiment 31A, wherein said plant extract is Trigonella foenum plant extract.

Embodiment 34A The composition according to any one of embodiments 1A to 33A, wherein the Niacinamide is from a source different from a Dunaliella alga.

Embodiment 35A The composition according to any one of embodiments 1A to 34A, wherein the Niacinamide is synthetic.

Embodiment 36A The composition according to any one of embodiments 1A to 35A, wherein the concentration of the Niacinamide in said composition is between about 0.02% w/w to about 5.0% w/w.

Embodiment 37A The composition according to any one of embodiments 1A to 36A, wherein the concentration of the Niacinamide in said composition is between about 0.04% w/w to about 0.2% w/w.

Embodiment 38A The composition according to any one of embodiments 1A to 37A, wherein the concentration of the Niacinamide in said composition is 0.04% w/w or 0.10% w/w.

Embodiment 39A The composition according to any one of embodiments 1A to 38A, wherein said composition comprises about 0.20% w/w to about 2.5% w/w Dead Sea extract, about 0.5% w/w to about 3.0% w/w Dunaliella Salina extract, about 5 ppm to about 50 ppm β-carotene and about 0.04% w/w to about 0.2% w/w Niacinamide.

Embodiment 40A The composition according to embodiment 39A, wherein said composition comprises about 0.5% w/w Dead Sea extract, about 3.0% w/w Dunaliella Salina extract, about 15.6 ppm β-carotene and about 0.1% w/w Niacinamide.

Embodiment 41A The composition according to embodiment 40A, wherein said composition comprises about 0.5% w/w Dead Sea water, about 3.0% w/w Dunaliella Salina extract, about 15.6 ppm β-carotene and about 0.1% w/w Niacinamide.

Embodiment 42A The composition according to embodiment 39A, wherein said composition comprises about 0.2% w/w Dead Sea extract, about 1.2% w/w Dunaliella Salina extract, about 6.24 ppm β-carotene and about 0.04% w/w Niacinamide.

Embodiment 43A The composition according to embodiment 42A, wherein said composition comprises about 0.2% w/w Dead Sea water, about 1.2% w/w Dunaliella Salina extract, about 6.24 ppm β-carotene and about 0.04% w/w Niacinamide.

Embodiment 44A A composition for enriching a skin cell with at least one Vitamin A, the composition comprising at least one Dead Sea extract and a combination of β-carotene and Niacinamide, wherein the combination comprises an amount of each of β-carotene and Niacinamide sufficient to induce intercellular production of at least one Vitamin A upon application of said composition onto at least a region of a skin of a subject, thereby enriching a skin cell with at least one Vitamin A.

Embodiment 45A The composition according to embodiment 44A, wherein said composition further comprises at least one extract of Dunaliella alga.

Embodiment 46A The composition according to embodiment 45A, wherein said at least one extract of Dunaliella alga is selected from Dunaliella Salina extract, Dunaliella Bardawill extract or a combination thereof.

Embodiment 47A The composition according to any one of embodiments 44A to 46A, wherein the concentration of the β-carotene in said composition is at least about 1 ppm and the concentration of the Niacinamide in said composition is at least about 0.02% w/w.

Embodiment 48A The composition according to any one of embodiments 44A to 47A being a composition according to any one of embodiments 1A to 43A.

Embodiment 49A The composition according to any one of embodiments 44A to 48A, wherein said composition comprises about 0.20% w/w to about 2.5% w/w Dead Sea extract, about 0.5% w/w to about 3.0% w/w Dunaliella Salina extract, about 1 ppm to about 50 ppm β-carotene and about 0.04% w/w to about 0.2% w/w Niacinamide.

Embodiment 50A The composition according to embodiment 49A, wherein said composition comprises about 0.5% Dead Sea extract, about 3.0% w/w Dunaliella Salina extract, about 15.6 ppm β-carotene and about 0.10% w/w Niacinamide.

Embodiment 51A The composition according to embodiment 49A, wherein said composition comprises about 0.2% w/w Dead Sea extract, about 1.2% w/w Dunaliella Salina extract, about 6.24 ppm β-carotene and about 0.04% w/w Niacinamide.

Embodiment 52A The composition according to any one of embodiments 44A to 51A, wherein said Vitamin A is selected from retinol, retinoic acid, retinal or any combination thereof.

Embodiment 53A The composition according to any one of the preceding embodiments, being a composition selected from a skin-care and pharmaceutical composition.

Embodiment 54A The composition according to embodiment 53A, wherein said composition is for topical application.

Embodiment 55A The composition according to embodiment 54A, being a synergistic composition.

Embodiment 56A The composition according to any one of the preceding embodiments, being in the form selected from a lotion, an ointment, a gel, a mask, a toner, an essence, a cream, a water in oil or oil in water emulsion, a shampoo, a moisturizer, a sunscreen, a cream, a stick, a spray, an aerosol, foam, a paste, a mousse, a solid, semi-solid, or a liquid make-up, a foundation, and an eye make-up.

Embodiment 57A The composition according to any one of the preceding embodiments, further comprising at least one additive selected from a diluent, a preservative, an abrasive, an anticaking agent, an antistatic agent, a binder, a buffer, a dispersant, an emollient, an emulsifier, a co-emulsifiers, a fiberous material, a film forming agent, a fixative, a foaming agent, a foam stabilizer, a foam booster, a gellant, a lubricant, a moisture barrier agent, a plasticizer, a preservative, a propellant, a stabilizer a surfactant, a suspending agent, a thickener, a wetting agent, and a liquefier.

Embodiment 58A The composition according to any one of the preceding embodiments, further comprising at least one additive selected from an anti-acne agent, an anti-aging agent, an antibacterial agent, an anti-cellulites agent, an antidandruff agent, an antifungal agent, an anti-inflammatory agent, an anti-irritation agent, an antimicrobial agent, an antioxidant agent, an antiperspirant agent, an antiseptic agent, a cell stimulant, a cleansing agent, a conditioner, a deodorant, a depilatory, a detergent, an enzyme, an essential oil, an exfoliant, a fungicide, a glosser, hair conditioner, hair set resin, hair sheen agent, hair waving agent, a humectants, a moisturizer, an ointment base, a perfume, a protein, a skin calming agent, a skin cleanser, a skin conditioner, a skin healing agent, a skin lightening agent, a skin protectant, a skin smoothing agent, a skin softening agent, a skin soothing agent, a sunscreen agent, a tanning accelerator, vitamins, a colorant, and a flavoring agent.

Embodiment 59A The composition according to any one of embodiments TA to 58A for use in the preparation of a cosmetic/skin-care or pharmaceutical composition.

Embodiment 60A The composition according to any one of embodiments TA to 59A, wherein said composition is for use in enriching a skin cell with hyaluronic acid.

Embodiment 61A The composition according to any one of embodiments 1A to 60A, wherein said composition is for one or more of protecting and/or improving the state of the skin, and preventing and/or treating imperfections of the skin of a subject.

Embodiment 62A The composition according to embodiment 61A, wherein the composition is for treating rings under the eye, symptoms of aging, protecting the skin, increasing the detoxification of xenobiotics, intervening on pigmentation level, inhibiting melanogenesis, protecting the body against pollution, stimulating the detoxification systems, stimulating hair and body hair growth, modulating DHT levels, intervening on adipocytes, and/or promoting lipolysis.

Embodiment 63A The composition according to embodiment 61A, wherein said composition is for treating or preventing at least one disease or disorder of the skin.

Embodiment 64A The composition according to embodiment 63A, wherein said disease or disorder of the skin is a secondary condition, being related to an existing condition.

Embodiment 65A The composition according to embodiment 64A, wherein said secondary condition is inflammation.

Embodiment 66A A lotion, an ointment, a gel, a mask, a toner, an essence, a shampoo, a moisturizer, a sunscreen, a cream, a stick, a spray, an aerosol, foam, a paste, a mousse, a solid, semi-solid, or a liquid make-up, a foundation, or an eye make-up comprising a composition according to any one of embodiments 1A to 65A.

Embodiment 67A The composition according to any one of embodiments 1A to 65A for use in the treatment and/or prevention of one or more disease or disorder, wherein said disease or disorder is associated with and/or is mediated by and/or is affected by and/or is related to one or more of biological pathways beings selected from cell cycle (KEGG, Kyoto Encyclopedia of Genes and Genomes); signaling pathways regulating pluripotency of stem cells (KEGG); nucleotide excision repair (KEGG); P53 signaling pathway (KEGG); assembly of collagen fibrils and other multimeric structures (WIKI); Elastic fiber formation (WIKI); keratinization (WIKI); sphingolipid metabolism (KEGG); NF KAPP B signaling pathway (KEGG); TNT signaling pathway (KEGG); apoptosis (KEGG); base excision repair (KEGG); nod-like receptor signaling pathway (KEGG); Yersinia infection (KEGG); or any combination thereof.

Embodiment 68A The composition according to any one of embodiments 1A to 65A for use in a method of one or more of protecting and/or improving the state of the skin of a subject, and preventing and/or treating imperfections of the skin of a subject in need thereof, said method comprising topically administering a composition according to any one of embodiments 1 to 65 onto the skin of said subject.

Embodiment 69A The composition according to embodiment 68A, wherein the imperfections of the skin of a subject is due to deficiency of retinol.

Embodiment 70A The composition according to embodiment 68A or 69A, wherein the protecting and/or improving the state of the skin of a subject, and preventing and/or treating imperfections of the skin of a subject in need thereof is mediated by at least one Vitamin A.

Embodiment 71A The composition according to any one of embodiments 68A to 70A, for treating rings under the eye, symptoms of aging, protecting the skin, increasing the detoxification of xenobiotics, intervening on pigmentation level, inhibiting melanogenesis, protecting the body against pollution, stimulating the detoxification systems, stimulating hair and body hair growth, modulating DHT levels, intervening on adipocytes, and/or promoting lipolysis.

Embodiment 72A The composition according to any one of embodiments 1A to 65A for use in a method for treating or preventing a disease or disorder of the skin of a subject, said method comprising administering to a subject in need thereof a composition according to any one of embodiments 1 to 65.

Embodiment 73A The composition according to embodiment 72A, wherein said disease or disorder of the skin is a secondary condition, being related to an existing condition.

Embodiment 74A The composition according to any one of embodiments 1A to 65A for use in a method of enriching a skin of a subject with hyaluronic acid, said method comprising topically administering a composition according to any one of embodiments 1A to 65A onto the skin of said subject.

Embodiment 75A A method for treating and/or preventing one or more disease or disorder, the method comprises administration of the composition according to any one of embodiments 1A to 65A to a subject in need thereof, wherein said disease or disorder is associated with and/or is mediated by and/or is affected by and/or is related to one or more of biological pathways beings selected from cell cycle (KEGG, Kyoto Encyclopedia of Genes and Genomes); signaling pathways regulating pluripotency of stem cells (KEGG); nucleotide excision repair (KEGG); P53 signaling pathway (KEGG); assembly of collagen fibrils and other multimeric structures (WIKI); Elastic fiber formation (WIKI); keratinization (WIKI); sphingolipid metabolism (KEGG); NF KAPP B signaling pathway (KEGG); TNT signaling pathway (KEGG); apoptosis (KEGG); base excision repair (KEGG); nod-like receptor signaling pathway (KEGG); Yersinia infection (KEGG); or any combination thereof.

Embodiment 76A A method of enriching a skin cell with at least one Vitamin A the method comprising:
applying onto at least a region of a skin of a subject a composition comprising at least one Dead Sea extract and a combination of β-carotene and Niacinamide, wherein the combination comprises an amount of each of β-carotene and Niacinamide sufficient to induce intercellular production of Vitamin A thereby enriching the skin cell with retinol.

Embodiment 77A The method according to embodiment 76A, wherein said composition further comprises at least one extract of Dunaliella alga.

Embodiment 78A The method according to embodiment 77A, wherein said at least one extract of Dunaliella alga is selected from Dunaliella Salina extract, Dunaliella Bardawill extract or a combination thereof.

Embodiment 79A The method according to any one of embodiments 76A to 78A, wherein the concentration of the β-carotene in said composition is at least about 1 ppm and the concentration of the Niacinamide in said composition is at least about 0.02% w/w.

Embodiment 80A The method according to any one of embodiments 76A to 79A, wherein said composition being a composition according to any one of embodiments 1A to 65A.

Embodiment 81A The method according to any one of embodiments 76A to 79A, wherein said composition comprises about 0.20% w/w to about 2.5% w/w Dead Sea extract, about 0.5% w/w to about 3.0% w/w Dunaliella Salina extract, about 1 ppm to about 50 ppm β-carotene and about 0.04% w/w to about 0.2% w/w Niacinamide.

Embodiment 82A The method according to embodiment 81A, wherein said composition comprises about 0.5% Dead Sea extract, about 3.0% w/w Dunaliella Salina extract, about 15.6 ppm β-carotene and about 0.1% w/w Niacinamide.

Embodiment 83A The method according to embodiment 82A, wherein said composition comprises about 0.2% w/w Dead Sea extract, about 1.2% w/w Dunaliella Salina extract, about 6.24 ppm β-carotene and about 0.04% w/w Niacinamide.

Embodiment 84A The method according to any one of embodiments 76A to 83A, wherein said Vitamin A is selected from retinol, retinoic acid, retinal or any combination thereof Embodiment 85A A method of enriching a skin of a subject with hyaluronic acid, the method comprising applying onto at least a region of a skin of said subject a composition according to any one of embodiments 1A to 65A.

Embodiment 86A A method of inducing in-vivo production of retinol, the method comprising:
application onto at least a region of a skin of a subject a composition comprising at least one Dead Sea Extract, β-carotene and Niacinamide, wherein the amount of the β-carotene in said composition is sufficient to provide a source for retinol and wherein the amount of the Niacinamide in said composition is sufficient to assist retinol dehydrogenase enzyme in transforming retinal into retinol, thereby inducing production of retinol in at least one skin cell of said subject.

Embodiment 87A The method according to embodiment 86A, wherein the concentration of the β-carotene in said composition is at least about 1 ppm and the concentration of the Niacinamide in said composition is at least about 0.02% w/w.

Embodiment 88A The method according to embodiment 86A or 87A, wherein said composition further comprises at least one extract of Dunaliella alga.

Embodiment 89A The method according to any one of embodiments 86A to 88A, wherein said method comprises application onto at least a region of a skin of a subject a composition according to any one of embodiments 1A to 65A.

1. A composition comprising at least one Dead Sea extract, β-carotene and Niacinamide, wherein the concentration of the β-carotene in said composition is at least about 1 ppm and the concentration of the Niacinamide in said composition is at least about 0.02% w/w, and wherein said composition optionally further comprises at least one extract of Dunaliella alga.

2-4. (canceled)

5. The composition according to claim 1, wherein said Dead Sea extract is a mixture of natural materials obtained from the waters of the Dead Sea, the mud surrounding the Dead Sea and/or the soil bed of the Dead Sea.

6. The composition according to claim 1, wherein said Dead Sea extract is the saline waters obtained from the Dead Sea or is an aqueous solution simulating the salts and minerals content of the Dead Sea water.

7-21. (canceled)

22. The composition according to claim 1, wherein the β-carotene and/or the Niacinamide are is from a source different from a Dunaliella alga.

23-29. (canceled)

30. The composition according to claim 1, wherein the Niacinamide is from a source different from a plant extract, e.g., wherein said plant extract is one or both of Zizyphus and Trigonella foenum plant extract.

31-38. (canceled)

39. The composition according to claim 1, wherein said composition comprises about 0.20% w/w to about 2.5% w/w Dead Sea extract (e.g., about 0.5% Dead Sea extract), about 0.5% w/w to about 3.0% w/w Dunaliella Salina extract (e.g., about 3.0% w/w Dunaliella Salina extract), about 5 ppm to about 50 ppm β-carotene (e.g., about 15.6 ppm β-carotene) and about 0.04% w/w to about 0.2% w/w Niacinamide (e.g., about 0.1% w/w Niacinamide).

40-52. (canceled)

53. The composition according to claim 1, being a composition selected from a skin-care and pharmaceutical composition.

54. (canceled)

55. The composition according to claim 1, being a synergistic composition.

56-65. (canceled)

66. A lotion, an ointment, a gel, a mask, a toner, an essence, a shampoo, a moisturizer, a sunscreen, a cream, a stick, a spray, an aerosol, foam, a paste, a mousse, a solid, semi-solid, or a liquid make-up, a foundation, or an eye make-up comprising a composition according to claim 1.

67. (canceled)

68. A method of one or more of: protecting and/or improving the state of the skin of a subject; preventing and/or treating imperfections of the skin of a subject in need thereof; and treating or preventing a disease or disorder of the skin of a subject; said method comprising topically administering a composition according to claim 1 onto the skin of said subject.

69. The method according to claim 68, wherein the imperfections of the skin of a subject is due to deficiency of retinol.

70. The method according to claim 68, wherein the protecting and/or improving the state of the skin of a subject; preventing and/or treating imperfections of the skin of a subject in need thereof; and treating or preventing a disease or disorder of the skin of a subject, is mediated by at least one Vitamin A.

71. The method according to claim 68, for treating rings under the eye, symptoms of aging, protecting the skin, increasing the detoxification of xenobiotics, intervening on pigmentation level, inhibiting melanogenesis, protecting the body against pollution, stimulating the detoxification systems, stimulating hair and body hair growth, modulating DHT levels, intervening on adipocytes, and/or promoting lipolysis.

72-74. (canceled)

75. A The method according to claim 68, wherein said method is a method for treating and/or preventing one or more disease or disorder, wherein said disease or disorder is associated with and/or is mediated by and/or is affected by and/or is related to one or more of biological pathways beings selected from cell cycle (KEGG, Kyoto Encyclopedia of Genes and Genomes); signaling pathways regulating pluripotency of stem cells (KEGG); nucleotide excision repair (KEGG); P53 signaling pathway (KEGG); assembly of collagen fibrils and other multimeric structures (WIKI); Elastic fiber formation (WIKI); keratinization (WIKI); sphingolipid metabolism (KEGG); NF KAPP B signaling pathway (KEGG); TNT signaling pathway (KEGG); apoptosis (KEGG); base excision repair (KEGG); nod-like receptor signaling pathway (KEGG); Yersinia infection (KEGG); or any combination thereof.

76. A method of enriching a skin cell with at least one Vitamin A, the method comprising:
applying onto at least a region of a skin of a subject a composition comprising at least one Dead Sea extract and a combination of β-carotene and Niacinamide, wherein the combination comprises an amount of each of β-carotene and Niacinamide sufficient to induce intercellular production of at least one Vitamin A, thereby enriching the skin cell with at least one Vitamin A.

77-80. (canceled)

81. The method according to claim 76, wherein said composition comprises about 0.20% w/w to about 2.5% w/w Dead Sea extract (e.g., about 0.5% Dead Sea extract), about 0.5% w/w to about 3.0% w/w Dunaliella Salina extract (e.g., about 3.0% w/w Dunaliella Salina extract), about 1 ppm to about 50 ppm β-carotene (e.g., about 15.6 ppm β-carotene) and about 0.04% w/w to about 0.2% w/w Niacinamide (e.g., about 0.1% w/w Niacinamide).

82-83. (canceled)

84. The method according to claim 76, wherein said Vitamin A is selected from retinol, retinoic acid, retinal or any combination thereof.

85. A method of enriching a skin of a subject with hyaluronic acid, the method comprising applying onto at least a region of a skin of said subject a composition according to claim 1.

86. A method of inducing in-vivo production of retinol, the method comprising:
application onto at least a region of a skin of a subject a composition comprising at least one Dead Sea Extract, β-carotene and Niacinamide, wherein the amount of the β-carotene in said composition is sufficient to provide a source for retinol and wherein the amount of the Niacinamide in said composition is sufficient to assist retinol dehydrogenase enzyme in transforming retinal into retinol, thereby inducing production of retinol in at least one skin cell of said subject.

87. The method according to claim 86, wherein said composition comprises about 0.20% w/w to about 2.5% w/w Dead Sea extract (e.g., about 0.5% Dead Sea extract), about 0.5% w/w to about 3.0% w/w Dunaliella Salina extract (e.g., about 3.0% w/w Dunaliella Salina extract), about 1 ppm to about 50 ppm β-carotene (e.g., about 15.6 ppm β-carotene) and about 0.04% w/w to about 0.2% w/w Niacinamide (e.g., about 0.1% w/w Niacinamide).

88-89. (canceled)
